(19) United States
(12) Patent Application Publication
(10) Pub. No.: US 2006/0292647 A1
(43) Pub. Date: Dec. 28, 2006

(54) Reflex supplemental testing - A rapid, efficient and highly accurate method to identify subjects with an infection, disease or other condition

(76) Inventors: Lawrence R. Green, Redmond, WA (US); Anne L. Sherwood, Kirkland, WA (US)

Correspondence Address: FAEGRE & BENSON LLP;PATENT DOCKETING, 2200 WELLS FARGO CENTER, 90 SOUTH 7TH STREET, MINNEAPOLIS, MN, 55402-3901, US

(21) Appl. No.: 11/069,351

(22) Filed: Mar. 1, 2005

Related U.S. Application Data
(60) Provisional application No. 60/633,217, filed on Dec. 3, 2004.
(60) Provisional application No. 60/645,428, filed on Jan. 20, 2005.

(51) Int. Cl. G01N 33/554; G01N 33/569

(52) U.S. Cl. 435/7.32

(57) ABSTRACT

The present disclosure concerns methods, compositions and apparatus for detecting pathogens and/or molecular markers. In a particular embodiment, the pathogen to be detected may be Mycobacterium bovis or any other Mycobacterium species that causes tuberculosis in a mammal. However, the disclosed methods are not limited and virtually any type of pathogen and/or molecular marker may be screened and detected. Preferred embodiments comprise reflex supplemental testing using the same assay at approximately 100% sensitivity and the highest possible corresponding sensitivity—in one example 70%. Such assay conditions, used iteratively, result in elimination of 70% of uninfected subjects for each round of testing. Use of 4 or more rounds of testing results in less than 1% error. Since only positive samples are retested, the methods provide a rapid, inexpensive and highly accurate way to detect infected subjects.

### RELATED APPLICATIONS

The present application claims the benefit under 35 U.S.C. §119(e) of Provisional U.S. Patent Application Ser. No. 60/633,217, entitled “Reflex Supplemental Testing for Mycobacterium Bovis—A Rapid Method to Isolate and Identify Infected Animals,” filed Dec. 3, 2004, and Provisional U.S. Patent Application Ser. No. 60/645,428, entitled “Rapid Diagnostic Test for Mycobacterium bovis Infection,” filed Jan. 20, 2005, the entire contents of both applications of which are incorporated herein by reference.

### FIELD

The present invention relates to methods for detecting the presence of pathogens and/or marker molecules in biological samples. In a particular embodiment, the methods involve use of a highly sensitive and selective test for the presence of a pathogen and/or marker molecule. Because sensitivity can be selected to be at or close to 100%, a negative result is considered to be indicative of the absence of the pathogen or marker. In a more particular embodiment, samples exhibiting positive test results are subjected to reflex supplemental testing, using the same assay, with sensitivity set at or close to 100%. A second positive result may be used as an indication for a third round of testing. With each round of reflex testing, the number of false positive results is decreased. The presence of false positives may be eliminated or reduced to any desired level by selection of an appropriate number of iterative reflex tests, depending on number of subjects tested, sensitivity and specificity of the assay used. Use of a selected number of rounds of reflex testing of positive samples may result in virtual elimination of false positives.

### BACKGROUND

Bovine tuberculosis is an infectious and highly contagious disease in cattle, caused by infection with Mycobacterium bovis. It may be spread by aerosol exposure to Mycobacterium or by ingestion of contaminated material. The disease tends to progress as a chronic inflammation, that is relatively asymptomatic during the early stages. Advanced cases are characterized by nonspecific symptoms such as weakness, loss of appetite, lymph node swelling, persistent cough and respiratory distress and may be mistaken for other types of respiratory disease. Although the bacterium preferentially infects cattle, transmission to humans and other mammals has been reported.

Standard methods of detection presently include tuberculin skin testing. However, false negative or false positive results of the standard skin test are not uncommon. As a positive test is likely to result in destruction of the putatively infected animal, along with other herd animals that have been directly exposed to that animal, a need exists for a more sensitive and accurate method for tuberculosis testing. Such a method would also be of benefit for testing human subjects for tuberculosis and other diseases.

Reflex testing to reduce the incidence of false positive results has been recommended for such infectious diseases as hepatitis C viral infection, human immunodeficiency virus (HIV) infection and hepatitis B infection (e.g., Morbidity and Mortality Weekly Report, Dept. Health and Human Services, Centers for Disease Control and Prevention, Feb. 7, 2003, 52(RR-3):1-13). However, such previous reflex testing has been performed using a different type of assay than the one used to generate the initial positive result. For example, the Centers for Disease Control and Prevention (CDC) has recommended that an initial positive test result for the presence of anti-hepatitis C antibody be followed up with a reflex test using a more specific serologic test, such as recombinant immunoblot assay or a nucleic acid screening test (Id.) Because such tests are often more expensive, time consuming and require a greater degree of technical expertise, reflex testing using different, more sensitive assays may considerably increase the expense, delay, or difficulty of obtaining definitive test results. Previous examples of reflex testing have focused on the use of an initial test of lower accuracy but greater speed, economy or convenience, followed by a reflex test of higher accuracy, usually with slower speed, greater expense and more complicated testing procedures. Among other things, such testing protocols are plagued by the presence of false negatives, resulting in a residual pool of infected subjects that may spread the disease. A need exists for a reflex testing method that may repetitively utilize the same type of assay to reduce or eliminate false positive and false negative results.

The skilled artisan will realize that the reflex testing methods disclosed and claimed herein are not limited to tuberculosis in either bovines or humans, but rather may be applied to testing for the presence of a wide range of pathogens and/or marker molecules. A number of different tests for various pathogens and/or marker molecules are known in the art and commercially available. It is within the skill in the art to vary the conditions used to perform such tests to provide a higher or lower level of stringency, for example by varying parameters such as temperature, pH, number and/or stringency of wash steps, the presence, absence or concentration of agents such as detergents, chaotrophic agents (e.g., formamide, urea, guanidinium, isothiocyanate), chelating agents (e.g., EGTA, EDTA), compounds to reduce non-specific binding of antibodies or other detection moieties (e.g., bovine serum albumin), salt concentration, divalent cation concentration and other techniques known in the art. Using such techniques, the relative sensitivity and/or specificity of a given test may be adjusted. In many cases, the sensitivity and specificity vary inversely. That is, as the sensitivity of the assay is increased, the specificity is decreased due to non-specific binding or cross-reactivity with other, similar pathogens or molecules.

In preferred embodiments using the presently disclosed and claimed reflex testing methods, assay conditions may be selected to provide sensitivity at or close to 100%, with correspondingly reduced specificity. Such conditions may be selected to eliminate the presence of false negative results, allowing samples that give negative test results to be eliminated from further testing. False positives may be reduced or eliminated by iterative testing of only those samples giving positive test results in the previous testing cycle. The testing methods disclosed an claimed herein provide substantial advantages over prior art testing methods in terms of economy, speed, efficiency, simplicity of testing and reduction or elimination of false positives and false negatives.

### BRIEF DESCRIPTION OF THE DRAWINGS

The following drawings form part of the present specification and are included to further demonstrate certain embodiments of the present invention. The embodiments may be better understood by reference to one or more of these drawings in combination with the detailed description of specific embodiments presented herein.

FIG. 1 is an illustration of a CMOS image sensor.

FIG. 2 is an illustration of a Bayer color filter mosaic array.

FIG. 3 is an illustration of possible filter arrangements for an image sensor.

FIG. 4 is an illustration of microlens operation in an image sensor.

FIG. 5 is a graph of quantum efficiency of an exemplary image sensor.

FIG. 6 is a flow chart illustrating an embodiment of calibration for image sensor optimization.

FIG. 7 is a flow chart illustrating an embodiment of image sensor optimization.

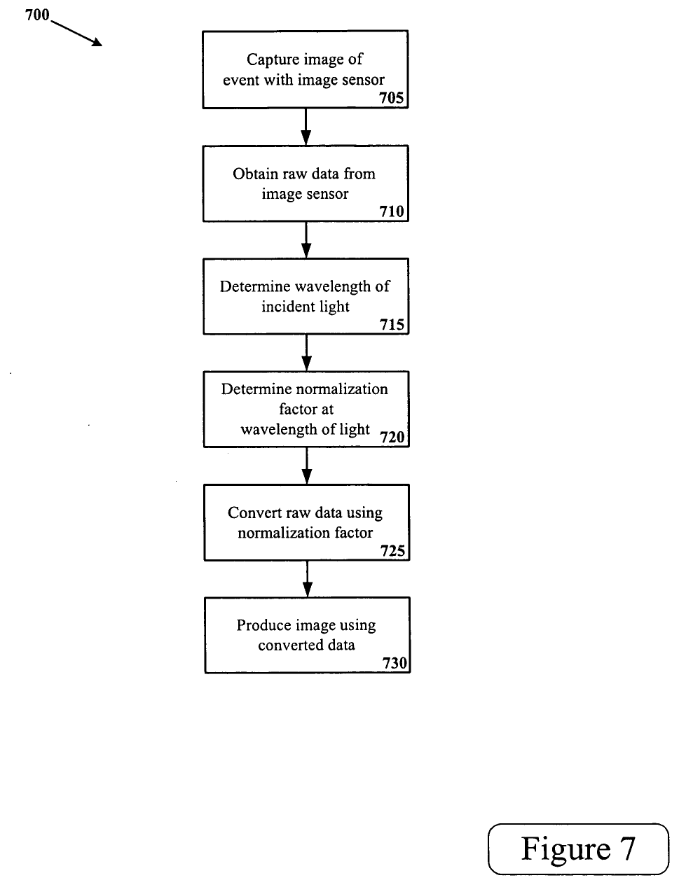

FIG. 8 is an illustration of light scattering detection.

FIG. 9 shows confirmed new cattle infections with bovine TB in Great Britain from 1996-2004. Data taken from Department for Environment Food and Rural Affairs, National Statistics, Feb. 23, 2005, (statistics.defra.gov.uk/esg/statnot/tbpn.pdf).

FIG. 10 illustrates an exemplary Reflex Supplemental Testing scheme, assuming normal distribution for infected and non-infected cattle.

FIG. 11 shows the effect of specificity and prevalence on the RST number in testing cattle for TB.

FIG. 12 illustrates an illustrative example of a Total Optical Assay Device (TOAD™).

FIG. 13 a schematic diagram, illustrating the components of an exemplary TOAD™ apparatus.

FIG. 14 shows a schematic of the upper surface of a TOAD™ apparatus with the casing 1410 removed to show the stage 1420.

FIG. 15 shows an exemplary crib set of 11 samples and a negative control reference, using ferrite CP10-ESAT6 fusion protein conjugate with positive and negative cattle sera samples. Samples were analyzed as disclosed in Example 1.

FIG. 16 shows an exemplary ROC curve.

FIG. 17 shows an exemplary assay result for autothreshold normalized relative luminosity screening and RST test on cattle with positive M. bovis infection.

FIG. 18 shows an exemplary comparison of PPV (Positive Predicted Values) for Caudal Fold, Bovigam and the presently disclosed Reflex Supplemental Testing methods.

### DESCRIPTION OF ILLUSTRATIVE EMBODIMENTS

Additional details illustrating exemplary embodiments of the present invention are disclosed in U.S. patent application Ser. Nos. 10/425,222, filed Apr. 29, 2003; 10/373,546, filed Feb. 24, 2003; 10/373,408, filed Feb. 24, 2003; 10/373,408, filed Feb. 24, 2003 and PCT Patent Application PCT/US2004/04675, filed Feb. 17, 2004, the text of each of which is incorporated herein by reference.

Definitions

Terms that are not otherwise defined herein are used in accordance with their plain and ordinary meaning.

As used herein, “a” or “an” may mean one or more than one of an item.

As used herein, “capture molecule” or “probe” refers to a molecule or aggregate that has binding affinity for one or more pathogens, pathogen associated molecules, and/or marker molecules. Within the scope of the present invention virtually any molecule or aggregate that has a binding affinity for some pathogen and/or marker molecule of interest may be a “probe.” “Probes” include, but are not limited to, polyclonal antibodies, monoclonal antibodies, antibody fragments, FAb fragments, humanized antibodies, single-chain antibodies, chimeric antibodies, affibodies, oligonucleotides, polynucleotides, nucleic acids, aptamers, binding proteins, receptor proteins, biotin, streptavidin, avidin and any other known ligand that can bind to at least one pathogen and/or pathogen associated molecule.

As used herein, a “marker” molecule refers to a molecule, or aggregate of molecules, whose presence, absence and/or concentration in a sample is indicative of the presence or absence of a disease, pathogenic organism or other condition. For example, the presence of particular forms of cancer in a subject may be indicated by the presence or elevated concentrations of various marker molecules, such as prostate specific antigen (PSA), CA125, mutant forms of ras or Her2-neu protein, CEA (carcinoembryonic antigen) and other molecules. Similarly, Alzheimer's disease may be indicated by the presence or elevated levels of amyloid beta peptide, amyloid precursor protein (APP) or other known markers. Many such marker molecules or molecular complexes are known in the art for different disease states or conditions and any such known marker may be assayed for using the claimed methods.

As used herein, a “pathogen” is any virus, bacterium, microorganism, molecule or molecular aggregate known in the art to be associated with an infectious disease. Non-limiting examples of pathogens are listed in Table 1.

As used herein, “quantum efficiency” means the fraction of light or photon flux that is utilized or contributes to current or signal output for an imaging device.

As used herein, “image sensor”, “imaging device”, or “imager” refers to a device for capturing an image. The term includes, but is not limited to, a CMOS (complementary metal oxide semiconductor) image sensor and a CCD (charge-coupled device) imager.

As used herein, “photon flux” means the energy of photons striking a surface, including the surface of an image sensor. The energy striking a surface may be measured in watts per cm² and correlates with the number of photons striking a unit area over a given period of time.

Testing for Bovine Tuberculosis

In an exemplary embodiment, the disclosed reflex testing methods may be used to test for the presence of tuberculosis causing bacteria in bovine or human subjects. Tuberculosis is a major international human and animal health issue, with the human disease causing approximately 3 million deaths annually (World Health Organization, “Tuberculosis control and research strategies for the 1990s. Memorandum from a W.H.O. meeting,” Bulletin W.H.O. 70:17-21, 1992.) Bovine tuberculosis poses a major cause of economic loss and a significant cause of zoonotic infection. (Dabom, C. J. and Grange, J. M., HIV/AIDS and its implication for the control of animal tuberculosis. 1993. Br. Vet. J. 149: 405-417.)

In cattle, tuberculosis (TB) is caused by infection with the bacterium, Mycobacterium bovis, a close relative of the human pathogen, Mycobacterium tuberculosis. Only very limited differences have been found in the antigens expressed by these strains, both of which are included in what has been defined as “the tuberculosis complex”, while clear differences in antigen expression distinguish these disease-causing agents from nonpathogenic strains. (Pollock, J. M. and P. Andersen, “Predominant recognition of the ESAT-6 protein in the first phase of interferon with Mycobacterium bovis in cattle,” Infect Immun, 1997, 65:2587-92; Andersen, A. B. et al., “Structure and function of a 40,000 MW protein antigen of Mycobacterium Tuberculosis,” Infect. Immun., 1992. 60:2317-2323; Harboe, M. T. et al., “Evidence for occurrence of the ESAT-6 protein in Mycobacterium tuberculosis and virulent Mycobacterium bovis and for its absence in Mycobacterium bovis BCG,” 1996, Infect. Immun.64:16-22.) Bovine tuberculosis can also infect humans, other domestic animals and some wildlife (including elk and whitetail deer).

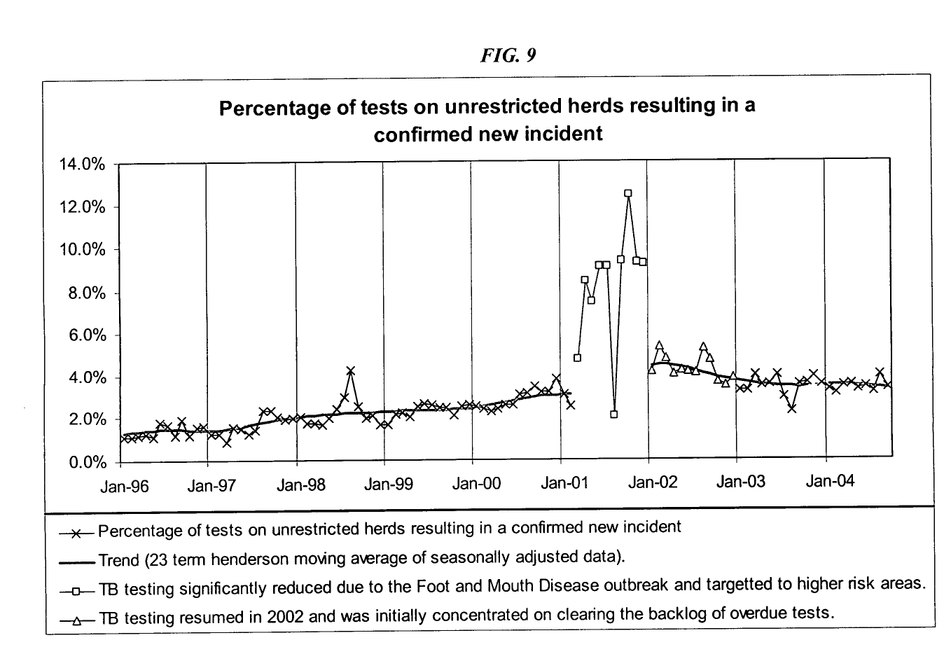

A bovine tuberculosis eradication program is currently in place in the US and in many other countries. Prevalence tracking began in 1917 and decreases in prevalence related to eradication programs result in benefits felt each year accruing with time. Estimated benefits in 2004 were $190 million per year. (Report of the Committee on Tuberculosis—2004, usaha.org/committees/reports/report-tb-2004.) Still, infections with bovine tuberculosis are frequently discovered. In fiscal year 2003, 10 affected cattle herds were detected and 6 more were detected in 2004. The source of infected dairy heifers/steers may be related to undetected TB in US dairies, mixing with infected feeder cattle or illegal movement from TB-infected areas. Inadequate animal identification may also hinder investigation efforts to identify the source of infection. Thirty-five tuberculosis cases were disclosed by slaughter surveillance in FY 2004 compared to the discovery of 39 cases in FY 2003. In Mexico the infection rate was 0.22/10,000 cattle imported in 2004 compared to 0.34/10,000 cattle in FY 2003. The percentage of tests on unrestricted herds in Great Britain resulting in a confirmed new infection incident in cattle from 1996 through to January 2004 is shown in FIG. 9, underscoring the need for better testing and eradication methods. Through October 2004 there were confirmed 1311 infected animals out of 372,284 animals tested (0.4% prevalence). Current approximate prevalence rates are 0.2% in the US, 0.4% in Mexico and 0.4% in Great Britain.

Humans contract bovine tuberculosis by: 1) inhaling air contaminated with the bacteria after an infected animal or infected person coughs or sneezes nearby; 2) drinking unpasteurized milk from an infected cow or eating raw or undercooked meat from an infected animal; or 3) handling infected meat in the dressing and processing of animal carcasses, especially if hands aren't washed carefully prior to consuming food.

However it is acquired, tuberculosis kills more people than any other disease. According to the World Health Organization (WHO), over 2 million people died in 2003 because of tuberculosis. Current estimates are that 1 in every 3 individuals in the world has latent tuberculosis, representing nearly 3 billion infected individuals. It is highly contagious, spreading rapidly akin to the common cold and can not be eradicated in the US unless it is controlled world-wide.

The symptoms of bovine tuberculosis in humans generally relate to the transmission method and are similar to those observed in tuberculosis (Mtb) infections. These symptoms include: cough, fever, night sweats, fatigue and weight loss. Patients infected with Mbov or Mtb have typically been identified using a tuberculin purified protein derivative (PPD) skin test and are treated with the same prescription drugs in either case. The drugs used to treat TB are by no means innocuous, contributing to morbidity and mortality because patients are required to endure treatment for prolonged periods for adequate therapy. It is the leading infectious killer worldwide because it can not easily be adequately treated. The prevalence of drug resistant TB is rising rapidly and is currently greater than 3%. It takes weeks to months to confirm that an individual is infected using the current best tests available.

There are no rapid diagnostic tests that will easily identify individuals who are infected and spreading the disease because the organism is difficult to isolate, slow growing, and unpredictably disseminated. The skin test requires great expertise to interpret, can not be used reliably to identify BCG vaccinated patients who could be infected or carry the organism, and fails to identify 30-40% of those individuals who are truly infected. A rapid (1 to 4 hours) and simple blood test would be of great value in finding those individuals who are infected and treating them to eradicate and or control TB.

Eradication of Tuberculosis

The control and eradication of tuberculosis poses a serious challenge due to the difficulty of identifying infected subjects. In most diseases, humans and animals readily develop detectable levels of antibodies in response to foreign proteins, which give a reliable indication of infection with a particular pathogen. Indeed, modem disease diagnosis and surveillance has generally been based on detection of antibodies against an infectious agent, rather than the disease agent itself.

However, the TB pathogen is one of a group of more insidious disease agents in which few detectable antibodies are observed by traditional methods until late in infection when the pathogen is well established and clinical signs of disease are already apparent. It has been demonstrated that most M. bovis infected cattle mount an effective cell-mediated immune (CMI) response, have a low antibody response and contain the infection within localized foci for long periods. (Theon, C. O. and Morris, J. A., The immune spectrum of Mycobacterium bovis infections in some mammalian species: a review. 1983, Vet. Bull, Weybridge, 53: 543-550.) For that reason, diagnostic tests measuring specific antibodies developed in response to TB infection have proven to be less sensitive than the tuberculin PPD skin test (in both humans and cattle), as they yield many false negatives or missed cases of TB. These serologic tests have typically also proven to have low specificity in that they frequently detect antibodies produced by the body in response to other comparatively harmless mycobacteria, giving rise to false positives.

Diagnostic Methods in Cattle

The current preferred method for diagnosing TB in cattle is the single intradermal tuberculin test (SIDT), a field test which measures the cell-mediated immune (CMI) response to M. bovis infection. Specifically, it measures the delayed type hypersensitivity reaction, a component of the CMI response, following intradermal inoculation of bovine tuberculin PPD. Results are assessed after 72 hours. (Wood, P. R and Jones, S. L. Bovigam™ an Internationally Accredited Diagnostic test for Bovine Tuberculosis. USAHA Proceedings, 1998, 1-9.) This test has been reported to have specificity as high as 96-99% in herds with widespread infection. (Monaghan, M., et. al., The tuberculin test. 1994, Vet. Micro. 40:111-124.)

However, the moderate sensitivity of the test (72 percent) is problematic in TB eradication programs because 28% of infected animals are incorrectly identified as uninfected. They are released back into the herd unless they are obviously ill, and will spread the infection to other uninfected cattle. Also, in some geographical areas, specificity is even further reduced because of significant cross-reactivity with atypical mycobacteria. Other infected animals not in the eradication program including white tail deer, elk, badgers, etc. also re-infect cattle previously cleared, thereby contributing to and compounding problems relating to eradication.

Both sensitivity and specificity have an important impact in eradicating the disease. With declining prevalence, the number of false positive animals detected relative to true positive animals rises due to less than 100 percent specificity. The positive predictive values decline in direct proportion to prevalence, resulting in most animals incorrectly identified as infected at low prevalence. Eradicating those animals incorrectly identified as infected is economically unacceptable. Nevertheless, in spite of these shortcomings, the skin test has been the only practical test available for screening large populations (human or animal) over the past 100 years.

Most cattle with tuberculosis do not exhibit clinical signs. They do, however, pose a serious threat to other livestock and humans. Tuberculosis prevalence is thought to be low in the US and many other countries where eradication programs are currently in process, but weakness in the program persists. In many countries a standard “in series” test is used, combining two less than perfect testing modalities. (M. Thrusfield, Veterinary Epidemiology, 2nd Edition, Blackwell Science, Malden, Mass., USA. 1995) The first test is a PPD skin test called the Caudal Fold Test (CFT) which is designed to identify either a suspect (reactor) or positive. All animals testing either positive or reactor are then retested with a Short Interval Comparative Tuberculin Test (SICTT), sometimes referred to as the comparative cervical test. Sensitivity for CFT ranges from 68-95% and specificity from 96 to 98.8%. SICTT has a sensitivity of 77-95% and a specificity greater than 99%. (Francis, J. et al., “The sensitivity and specificity of various tuberculin tests using bovine PPD and other tuberculins,” Veterinary Record, 1978 103:420-425; Monaghan, M L et al., “The tuberculin test,” Veterinary Microbiology, 1994, 40:111-124.) If this test is positive, the animal is slaughtered and tissues are collected for culture and examination by histopathology. By combining the two less than perfect tests the overall sensitivity is reduced to approximately 70% but specificity is nearly 100%, sparing incorrectly identified animals from being slaughtered. However, the reduced sensitivity results in some false negative results, with corresponding presence of cryptic infected animals resulting in a continued low level rate of infection. A good example of in series testing statistical outcome for diagnostic testing in series is discussed at prevmed.vet.ohio-state.edu/ext_{—}62b.htm.

CFP10-ESAT6 Fusion Protein

A molecular analysis was conducted of the genetic differences between virulent M. bovis and the attenuated vaccine strain M. bovis BCG. Three regions of difference designated RD1-RD-3 were identified using subtractive genomic hybridization. RD-1 was detected in all strains of M. tuberculosis and M. bovis, but is absent in all BCG substrains. (Maheiras, G.G. et al., 1996, “Molecular analysis of genetic differences between Mycobacterium bovis BCG and virulent M. bovis,” J. Bacteriol 178:1274-1282.) The ESAT-6 gene, encoding the Early Secreted Antigen Target 6 kD protein, is found within RD-1. ESAT-6 is a major T cell antigen which has been purified from M. tuberculosis short term culture filtrates. (Sorensen, A. L. et al., 1995, “Purification and characterization of a low molecular mass T-cell antigen secreted by M. tuberculosis,” Infect. Immun. 63:1710-1717; Harboe, M. et al, 1996, “Evidence for occurrence of the ESAT-6 protein in Mycobacterium tuberculosis and virulent Mycobacterium bovis and for its absence in Mycobacterium bovis BCG,” Infect. Immun. 64:16-22.) No function has yet been ascribed to ESAT-6, but it is strongly antigenic in nature, stimulates the production of gamma interferon from mice memory immune T lymphocytes and may contribute to the development of anti-tuberculosis immunity. (Andersen, et al., 1995, “Recall of long-lived immunity to Mycobacterium tuberculosis infection in mice,” J. Immunol. 154:3359-3372.)

A new gene which is cotranscribed with ESAT-6, designated lhp, has been identified. Within the lhp/esat-6 operon, a total of 13 potential genes (or open reading frames, ORFs) have been further identified, defining a novel gene family. The Mycobacterium tuberculosis lhp gene product has been identified as CFP-10, a low molecular weight 10 kD Culture Filtrate Protein, also found in short-term culture filtrates. (Berthet, F-X., et al., 1998, “A Mycobacterium tuberculosis operon encoding ESAT-6 and a novel low-molecular-mass culture filtrate protein (CFP-10),” Microbiology 144:3195-3203.) Thus, both CFP-10 and ESAT-6 are transcribed together early in M. tuberculosis or M. bovis and both encode small exported proteins. Since the two genes occur adjacent to one another in either genome, a fusion protein recombinant antigen composed of both full length proteins was designed as a capture probe for our assay.

Bovigam Tests

A new panel of tests are currently being evaluated in which killer T-cells, activated by the immune system following exposure to M. tuberculosis or M. bovis, are assayed for gamma interferon (IFN-γ) production when stimulated with specific bacterial epitopes from these organisms. The bovine application of this test is called Bovigam™, primate application is Primagam™ and human application is Quantiferon™. In these assays, whole blood is taken from the patient, and incubated overnight with tuberculin (proteins extracted from M. tuberculosis or M. bovis). If the patient (human or animal) has been previously exposed to TB, then the T cells in the blood will produce IFN-γ, detectable in a simple laboratory assay. The advantages of such a test is that extraction of the test blood sample does not interfere with the immune state of the subject so repeated testing is possible if necessary (the skin test requires a minimum gap of 60 days before retesting) and can be used to detect early cases of infection.

These tests can also be used reliably in HIV infected and other immuno-compromised individuals whereas skin tests cannot. Worldwide trials of well over 300,000 cattle have shown Bovigam™ to have a sensitivity ranging from 77 to 93.6% as compared to 65.6 to 84.4% for the skin test. If Bovigam™ and the skin test are used together then even higher sensitivity can be achieved.

Quantiferon Tests

Quantiferon™ has been tested in clinical trials involving over 3,000 individuals to date. In the improved version of Quantiferon™, QuantiFERON-TB GOLD test, M. tuberculosis antigens ESAT-6 or CFP-10 (gene products expressed in early in active infections of Mtb or Mbov) is are used in place of tuberculin PPD. ESAT-6 sequences have been shown to be recognized by antibodies in sera of tuberculous non-human primates (Kanaujia, G. V. et al., “Recognition of ESAT-6 Sequences by Antibodies in Sera of Tuberculous Nonhuman Primates,” Clin. and Diag. Lab. Immunol., 2004, 11(1):222-226) and cattle (Buddle, B. M., et al. Use of ESAT-6 in the interferon-gamma test for diagnosis of bovine tuberculosis following skin testing. Vet Microbiol, 2001. 80: 37-46; Buddle, B. M., et al. Use of mycobacterial peptides and recombinant proteins for the diagnosis of bovine tuberculosis in skin test positive cattle. The Veterinary Record, 2003. 615-620; Vordermeier, H. M., et al. Use of synthetic peptides derived from the antigens esat-6 and cfp-10 for differential diagnosis of bovine tuberculosis in cattle. Clin Diagn Lab Immunol, 2001. 8: 571-8) as well as humans (Arend, S. M., et al. Antigenic equivalence of human T-cell responses to Mycobacterium tuberculosis-specific RD1-encoded protein antigens ESAT-6 and culture filtrate protein 10 and to mixtures of synthetic peptides. Infect Immun, 2000. 68: 3314-21; Arend, S. M., et al. Detection of active tuberculosis infection by T cell responses to early secreted antigenic target 6-kDa protein and culture filtrate protein 10. J Infect Dis, 2000. 181: 1850-4; Berthet, F. X., et al. A Mycobacterium tuberculosis operon encoding ESAT-6 and a novel low molecular-mass culture filtrate protein (CFP-10). Microbiology, 1998. 144: 3195-203; Brock, I., et al. Performance of whole blood IFN-gamma test for tuberculosis diagnosis based on PPD or the specific antigens ESAT-6 and CFP-10. Int J Tuberc Lung Dis, 2001. 5: 462-7.)

A large number of published clinical studies have demonstrated the utility of measuring IFN-γ responses to ESAT-6 and/or CFP-10 (using methods other than Bovigam™ or Quantiferon™) for the detection of TB infection. (Dillon, D. C., et al. Molecular and immunological characterization of Mycobacterium tuberculosis CFP-10, an immunodiagnostic antigen missing in Mycobacterium bovis BCG. J Clin Microbiol, 2000. 38: 3285-90; Pathan A. A., et al. Direct ex vivo analysis of antigen-specific IFN-γ-secreting CD4 T cells in Mycobacterium tuberculosis-infected individuals: Associations with clinical disease state and effect of treatment. J Immunol, 2001. 167: 5217-25; Shams H., et al. Contribution of CD8+ T cells to gamma interferon production in human tuberculosis. Infect Immun, 2001. 69: 3497-501; Smith, S. M., et al. Human CD8(+) T cells specific for Mycobacterium tuberculosis secreted antigens in tuberculosis patients and healthy BCG-vaccinated controls in The Gambia. Infect Immun, 2000. 68: 7144-8; Vekemans J., et al. Tuberculosis contacts but not patients have higher gamma interferon responses to ESAT-6 than do community controls in The Gambia. Infect Immun, 2000. 69: 6554-7.) However, these studies have generally used testing systems that are complex to perform and unsuitable for routine diagnostic application (e.g. purified lymphocyte culture, ELISPOT). Trials in Australia have found the sensitivity and specificity of the Quantiferon™ test to be approximately 90% and 98% respectively. By comparison, the skin test has sensitivity in the order of 70% for detecting human TB infection.

Blood Vs. Skin Testing

While Bovigam™, Primagam™ and Quantiferon™ require only one blood draw, and are far more accurate than skin tests and TB antibody detection tests to date, they are considerably more expensive to run, tedious to perform as they are not automated and results are not available until up to 24 hours after incubation of activated T-cell lymphocytes with M. bovis or M. tuberculosis specific antigens. Furthermore, a fresh blood sample is required for each test and must be assayed within 24 hours of collection. Thus, these tests are impractical to use for widespread screening, and are best employed as confirmatory assays of samples testing positive by a simpler initial screening method.

In Great Britain the Intradermal Comparative Cervical Tuberculin test (SICCT/skin test) is the only test used to diagnose TB in cattle. Because the Bovigam test is considered to be more sensitive but less specific than the SICCT test, the possibility of using Bovigam to eradicate infected herds was investigated. Both conventional PPD antigens and newer synthetic antigens were used in a cost-effectiveness study to determine if the IFN test would be of use. (defra.gov.uk/animalh/tb/forum/papers/tbf71.pdf) It was observed that blood testing did result in detection before skin testing became positive, but the overall benefit economically could not be justified because of the complexity of the test and overall cost per animal.

Advantages related to blood testing over skin testing include;
Reducing the length of time a herd is under movement restriction
Removing the need to visit stock twice, as required for skin tests
Reducing veterinary and labor fees by reducing the time spent on testing
Reducing the number of rounds of testing required clearing up infection due to the test's increased sensitivity
Detecting cattle infection at an earlier stage of infection
Ability to re-test in very short intervals without interference as is a problem in skin testing (60 day minimal interval)
High sensitivity ideally suited to pick up more disease when infection is spreading rapidly
Distinguish between cattle vaccinated with BCG and cattle infected with M. bovis
Analysis of results is more precise rather than a subjective assessment of a skin reaction by a trained veterinary surgeon

The skin test (SICCT or CFT) requires the mustering of animals to administer the test and again to read results 2-3 days later. It is highly recommended that this be done by a trained veterinarian as the sensitivity of the test can be as low as 20% with inexperienced technicians. It costs much more in time and money to use experienced and trained veterinarians to administer and read the test than serum-based testing.

Although there has been a long and persistent search for serological assays which can detect circulating antibody to M. bovis, none to date have demonstrated the adequate sensitivity or specificity which would make them suitable as diagnostic tests for routine use. (Wood, P. R. and Rothel, J. S. In vitro immunodiagnostic assays for bovine tuberculosis. 1994, Vet. Micro, 40: 125-135)

Reflex Supplemental Testing—A Rapid, Efficient and Highly Accurate Method to Identify TB-Infected Subjects

The PriTest Rapid Diagnostic Test for Mycobacterium bovis (Mbv) infection provides an ideal serological method for widespread screening of TB infection in cattle. It is superior to the skin test in a number of ways, requiring a small serum sample (as little as 0.1 ml serum from a single blood draw will provide more than enough material for multiple rounds of testing). The test yields highly accurate results in less than 1.5 hours for an initial screen, and does not require a high level of technical expertise. A single blood sample is all that is required and the results are then processed in the laboratory where results may be automatically interpreted by instrumentation and software.

The test is economical to use in screening large numbers of cattle, especially since the high accuracy inherent in the test design minimizes the number of animals that have to be retested. With reflex supplemental testing (RST) the final results are obtained without having to rely on the more expensive and complex testing characterized by Bovigam. And the blood sample can be retested at any time, having none of the time dependence requirements (15 hours) required for accurate Bovigam testing.

Immune reactivity is not affected in the PriTest Rapid Diagnostic Test since there is no tuberculin injection and the results are obtained in the laboratory within a single day (including the initial screen and 2 confirmatory reflex supplemental tests on positive samples). As with Bovigam™, the PriTest Rapid Diagnostic Test is specific for M. bovis and provides a differential diagnosis between M. bovis and other mycobacterial infections such as M. paratuberculosis. Neither test reacts positively with BCG vaccinated animals. Bovigam™ requires a fresh blood sample with special handling requirements that must be tested within 15 hours. PriTest's Rapid Diagnostic Test needs only serum to test and requires no special handling so that samples may be collected and even frozen to be tested at a later date.

A typical test involves using a recombinant fusion protein capture probe of Mtb CFP-10/ESAT-6 which has been conjugated to ferrite particles coated with a carboxyl acrylic polymer. A defined quantity of beads is dispersed in a test serum sample which has been diluted 1:50 in a dilution buffer composed of 10 mM PBS, 0.1% BSA and 0.05% ProClin. The diluted sera and beads are incubated 15 minutes at room temperature (RT). Beads are then affixed to the walls of the sample chamber using neodymium magnets and re-dispersed in wash buffer and collected twice more. In preferred embodiments, a pair of spherical magnets may be arranged in a plastic block, one on either side of a sample chamber holder (such as a microfuge tube holder). Spherical magnets have been found to provide a more intense localized magnetic field for more efficient collection of ferrite beads from solution.

A secondary biotinylated anti-bovine antibody is used to label bovine TB antibodies that have been captured by recombinant antigen on the ferrite beads. After a 10 minute RT incubation, the beads are again affixed to the side walls of a chamber, such as a 1.5 ml Eppendorf microfuge tube, using magnets and twice more dispersed in wash buffer and recollected. The beads with biotinylated antibody affixed to bovine antibody are then incubated for 5 minutes with horseradish peroxidase streptavidin (HRP-SA) and then twice dispersing and washing the beads.

The beads are then suspended in 1:1 luminol peroxide solution and chemiluminescence is detected in a photoimager, for example, a PriTest TOAD™ (see e.g., Provisional U.S. Patent Application 60/540,720, filed Jan. 29, 2004, incorporated herein by reference). For example, an image may be obtained in a totally dark chamber for 10 minutes to accurately measure the photon image that corresponds to the level of antibody bound to beads. A software package may be used (e.g., Slider ver 3.6, see U.S. patent application Ser. No. 10/373,408, filed Feb. 24, 2003) to process up to 12 samples in a single pass. Assigned values of positive or negative are computed based on the reference negative control sera.

Each crib set currently has as many as 11 test samples in addition to the negative control. Observed signals, given as numerical values, are entered into an automated program which calculates relative luminosity (RL) of each sample by subtracting background of the crib from the observed sample value. The delta relative luminosity (ΔRL) for each sample is then calculated by subtracting RL of the negative control (consisting of a pooled sample of ˜10 bovine sera which have tested negative for TB) from the RL of each sample with a statistical adjustment for setting the threshold criteria that a sample must have to read positive.

A threshold, or cutoff value, for designating a sample as negative or positive for M. bovis antibodies, is determined by a preset algorithm contained within the program. Samples testing positive in the first round of screening are retested in a second round (Reflex Test #1). Samples testing positive in this second round are again retested in a confirmatory round (Reflex #2). Any samples testing negative at any point in the reflex study are confirmed as negative. Samples testing positive after Reflex #2 are confirmed positive in the assignment.

No single test will ever consistently produce the level of Sensitivity and Specificity required to effectively isolate infected animals with 100% certainty. Reflex supplemental testing involves testing deliberately at a threshold where false positives (i.e., at or near 100% sensitivity) are expected because the threshold for detecting positives has been set sufficiently low to catch 100% of positively infected animals.

By then testing in a second batch all of those animals that tested positive for infection again using the same assay, a second batch of uninfected animals will safely be released. The reflex supplemental testing process continues in steps, retesting at each interval only those animals testing positive, until only the true positive isolates are confirmed. This method of testing rapidly centers on infected animals while allowing animals that tested falsely positive to retest negative if indeed they are not infected. It is more efficient than a single screening test at accurately confirming those animals that are infected.

Calculating the RST Number

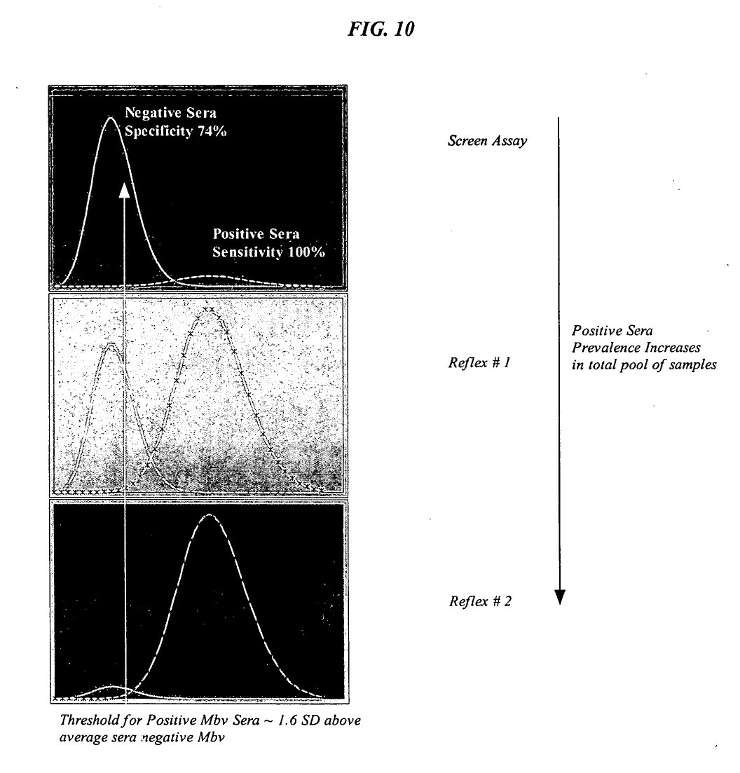

How many reflex supplemental tests are needed to with confidence stop retesting? Too many tests are of no value because time, money and resources are then wasted. This number can be easily calculated. Both seropositive and seronegative animals will have a test value that can be easily determined in an assay. A normal distribution curve of values for both the infected (positive) and uninfected (negative) animals can be determined (see FIG. 10).

The distribution curves do not need to be symmetrical (as shown) and do overlap in practice as shown in FIG. 10. If the two curves for positive and negative do not overlap, a threshold between the curves will easily differentiate between the two populations of infected and uninfected cattle with 100% specificity and sensitivity. But even if the curves overlap, as is the case with TB in cattle, a threshold may be chosen that is to the left (lower in value) of all infected (positive) animals tested. With that threshold assignment, 100% of all truly positive animals will be accurately detected (i.e., false negatives will be zero) even though some of the uninfected cattle will be falsely identified as positive as can be seen by examining FIG. 10 (i.e., a portion of the “negative sera” curve lies to the right of the threshold value). However, as can be see, with each iteration of reflex testing the number of false positive results decreases rapidly.

The assay value for each animal (either infected or uninfected) will with each test, and each reflex supplemental test, fall within one of the two curves shown in FIG. 10 and can be assigned as a positive or negative result, based on the threshold used in the assay. The value will be expected to fall anywhere under the appropriate curve (infected or uninfected) and may not necessarily with great precision reproduce the previous value measured. Variance is expected, as defined by the shape of the distribution curve.

The optimal RST number (N, equal to the number of cycles of iterative testing) depends on three factors—the total number of samples for processing (X), the number of truly infected cattle (prevalence) in a tested number of samples, and the specificity for a threshold that is sufficiently low to accurately assign all truly infected cattle a “positive” value. With each test, a percentage of the samples that are actually uninfected will be removed from the pool of samples in line for testing. Reflex supplemental testing results in each cycle enriching the percentage of positives relative to negatives that are ready for subsequent tests. This procedure is equivalent to increasing prevalence, making the positive predictive value of the test better with each re-test in the process and is shown in FIG. 10 in the change in relative distributions from Screen to Reflex # 1, to Reflex # 2, etc. The proportion of positives in the pool increases relative to the negatives in the pool with each cycle of testing. An optimized RST minimizes the false negative tests relative to the positive total tested. The total number of tests for optimization can be calculated as a sum:
Total Number of Tests(Screen+RSTs)=X{1+F+F²+F³+ . . . F^{N}}
Where F is the false positive factor dependent on the specificity
F=1−Specificity
Number of false positives=F{(X−true positive #)}
X=total sample process number
N=Optimized RST Number

We can optimize with a reasonable specificity by setting the minimal criteria to eliminate 1 false positive animal assuming there is a prevalence of 1 infected animal in the total number of samples being tested. (Assuming that the specificity is substantially greater than 50%, as is the case with the exemplary protocols disclosed herein.) This is equivalent to setting the last series in the sum to a value of 1, and that allows for the calculation of N based on total number of samples (X) and specificity.
XF^{N}=1
N={Log 1−Log X}/Log F

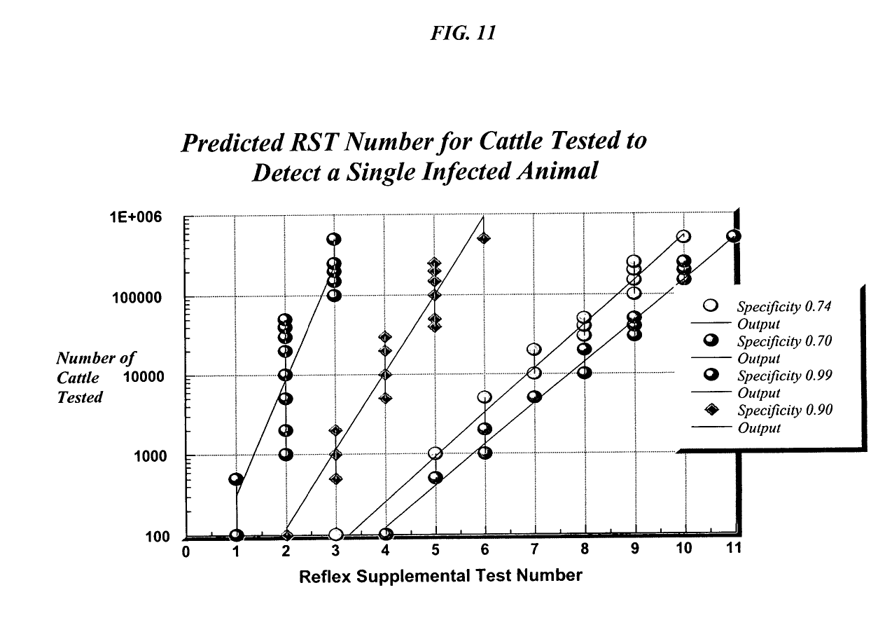

The specificity, prevalence, and number of initial tests in a screen will determine the total number of reflex supplemental tests required to with confidence assign cattle as infected or clear of infection for a given assay at 100% sensitivity. The RST number is calculated in Table 2 for 0.70, 0.74 (exemplary study), 0.90 and 0.99 specificities for 1 truly positive animal in a sample of from 100 to 500,000 cattle as shown in FIG. 11. It is apparent that the higher the specificity, the fewer total tests are required, but nevertheless the RST method quickly identifies within 3 to 4 cycles the truly infected animal in a 100 animal herd.

Optimizing the RST number reveals that a threshold about 1.6 standard deviations above the average for seronegative (uninfected) cattle will yield at 100% sensitivity a specificity of 74% in the current PriTest Rapid Mbv cattle assay. In a single screen and 3 reflex supplemental tests, the positively infected animal should be identified to within 1 of 4 animals in a herd of 1000 cattle, and could be positively identified by an additional 4 tests. Under these conditions, the total number of tests performed to positively identify the single infected animal in a 1000 animal herd is 1348. Thus, it can be seen that even with multiple iterative cycles of reflex testing, the total number of tests required is relatively low compared to the herd size.

Apparatus for Pathogen or Marker Detection

Certain embodiments of the present invention concern apparatus of use for pathogen and/or marker molecule detection assays. Additional details illustrating exemplary embodiments of the present invention are disclosed in U.S. patent application Ser. Nos. 09/974,089, filed Oct. 10, 2001; 10/035,367, filed Oct. 9, 2002; 10/425,222, filed Apr. 29, 2003; 10/373,546, filed Feb. 24, 2003; 10/373,408, filed Feb. 24, 2003; and provisional patent application 60/540,720, filed Jan. 29, 2004, the text of each of which is incorporated herein by reference in its entirety.

Total Optical Assay Device (TOAD)

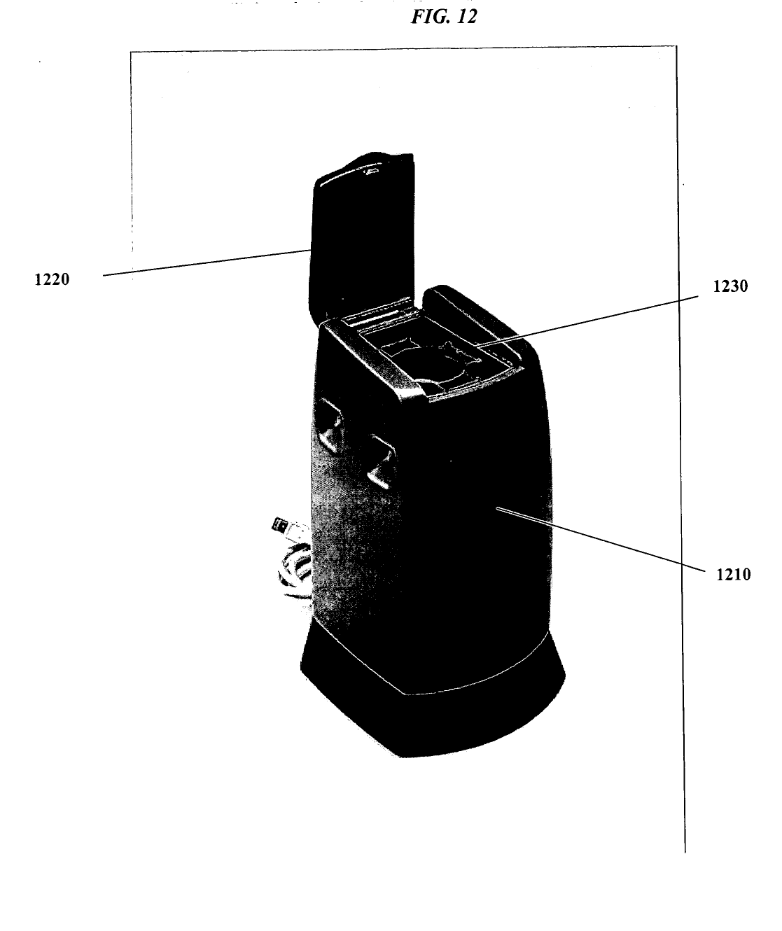

FIG. 12 illustrates a non-limiting exemplary embodiment of a Total Optical Assay Device (TOAD™). In this example, the TOAD™ comprises a light-tight casing 1210 with a hinged lid 1220. When the lid 1220 is closed, the casing 1210 blocks external light from the interior of the TOAD™, allowing highly sensitive detection of bioluminescence or other emitted light from samples to be analyzed. The lid 1220 is opened to show a stage 1230. Devices to be imaged, including but not limited to GRABBER™ slides (see below) or microtiter well devices, may be positioned on top of the stage 1230. Underneath the stage is a single focusing lens (not shown) that sits on top of a CCD (charge coupled device) chip or other light sensor (not shown). The slide or microtiter wells may be positioned on the stage 1230 by aligning them with corresponding shaped depressions on the top of the stage 1230. The casing 1210 may comprise depressions, handles or other features to facilitate moving the TOAD™.

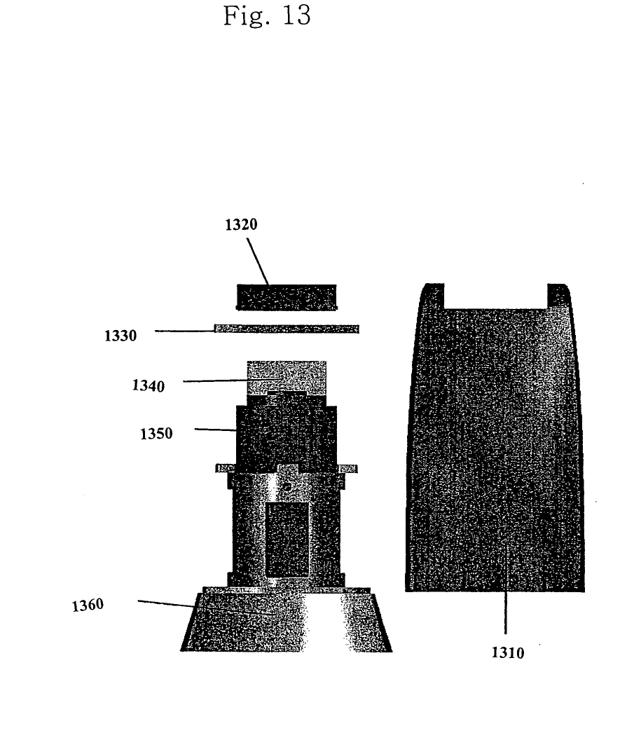

FIG. 13 illustrates a schematic view of an exemplary embodiment of a TOAD™, with the body of the casing 1310 displaced to show the internal components. The lid 1320 overlies a stage 1330, which is used to position slides, microtiter wells or other devices over a CCD chip located at the top of an imaging device 1350. In a non-limiting example, the imaging device 1350 is a QICAM 10-bit mono cooled CCD camera (Qimaging™, Burnaby, B. C., Canada, catalog #QIC-M-10-C). However, the TOAD™ is not limited to the exemplary embodiment and it is contemplated that other known types of imaging devices 1350 may be utilized. A single focusing lens (not shown) is interposed between the CCD chip and the stage 1330 and may be attached to the top of the imaging device 1350, for example using a C-mount adaptor.

In the exemplary embodiment the imaging device 1350 contains, in addition to a CCD chip, a Pelletier heat exchange element surrounding the CCD chip. The rest of the imaging device 1350 contains electronic components for processing the signals detected by the CCD chip, along with connections for a power supply, software interface and external data port, such as a 6-pin firewire connector. The Pelletier heat exchanger removes heat from the CCD camera 1350 and releases it inside the casing 1310. In order to prevent the TOAD™ from overheating, a separate fan driven cooling device 1340 is positioned adjacent to the top of the imaging device 1350. The cooling device 1340 drives hot air out of the casing through a light-tight vent at the back of the casing 1310 (not shown) and circulates cool air through the casing 1310 interior. During standard operation, the cooling device 1340 operates efficiently enough that the CCD camera 1350 may be operated continuously without overheating. Overheating of the CCD camera 1350 results in substantial increase in background noise, producing random bursts of apparent light detection throughout the optical field of the CCD chip.

The imaging device 1350 rests on top of a base 1360 that holds the imaging device 1350 in position relative to the stage 1330. The bottom of the base 1360 is open to allow access to on on-off switch at the bottom of the imaging device 1350 and connections to power supplies (e.g. 110 volt AC electrical cord), firewire and any other external connections.

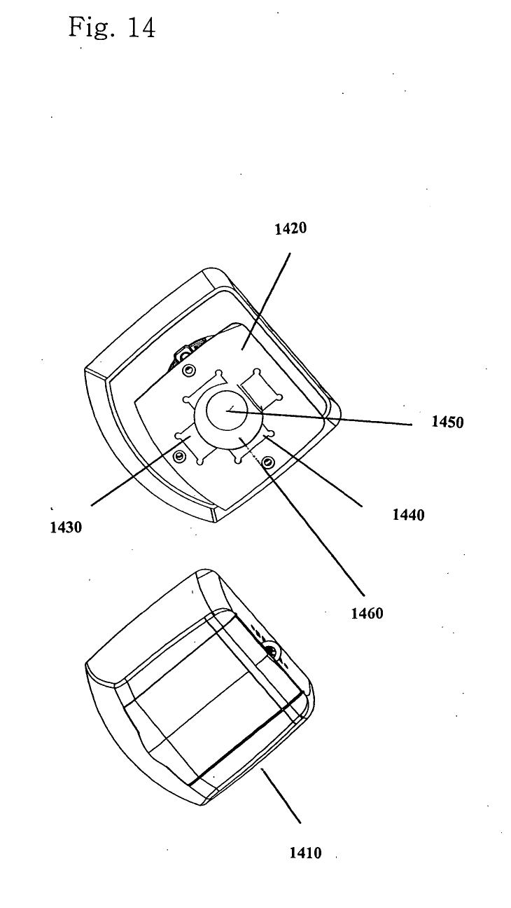

FIG. 14 is a schematic diagram showing the top view of an exemplary embodiment of a TOAD™, with the casing 1410 removed to show the upper surface of the stage 1420. Indentations 1430, 1440 are shown in the top of the stage 1420, arranged to allow positioning of slides, microtiter wells or other devices over the CCD chip. A hole 1460 in the center of the stage 1420 allows for light transmission through the slide, microtiter well or other device and detection by the underlying CCD chip 1450. It will be apparent to the skilled artisan that the use of transparent or translucent devices, as discussed in more detail below, facilitates the use of a TOAD™ apparatus with an optical detector located below the device to be optically read.

The exemplary embodiment discussed above is designed for optical detection with samples that spontaneously emit light, for example using any known chemiluminescent or bioluminescent detection system, such as the luminol system disclosed below. In this case, no external source of excitatory light is needed to detect bound analytes. The skilled artisan will realize that alternative systems may be utilized, such as fluorescently tagged detection molecules that bind to target analytes. Such systems are well known in the art. The use of a fluorescent detection system would necessitate additional components, such as an excitatory light source (e.g. laser, photodiode array, etc.), cutoff filters to screen the photodetector from excitatory light, and other such known components for fluorescent detection systems. The illustrative embodiment discussed above possesses the advantages of simplicity of construction and use, low cost, and sensitive detection of target analytes with minimal background noise.

Slides

In certain embodiments, optical detection may be used in combination with chips, matrix arrays and/or slides, such as microscope slides, as a binding surface for detection of pathogens and/or marker molecules. A variety of slide substrates are known, as discussed below.

Traditional Translucent Slides

Glass or plastic microscope slides have commonly been used as solid matrix supports for microarray analysis. Probe molecules have been attached to glass or plastic surfaces using cross-linking compounds. (See, e.g., Schena, Microarray Analysis, J. Wiley & Sons, New York, N.Y., 648 pp., 2002.) Probes may be printed as 2D arrays of spots. Many different kinds of cross-linkers are known, depending on the type of reactive moieties (e.g., sulfhydryl, amino, carboxyl, phenyl, hydroxyl, aldehyde, etc.) available on the probe molecules that can be cross-linked to the surface without affecting probe functionality (e.g., target molecule binding).

A problem with previous methods for probe attachment is that the capacity for attachment is limited. As probe size is increased, the number of possible binding sites for prospective target molecules is generally decreased. If the binding sites for the probe are saturated at a level below the threshold for detection, a signal will not be observed even if binding has occurred between probe and target molecule.

Attempts have been made to attach probes to glass surfaces using avidin-coated slides and biotin-conjugated probe molecules. Alternatively, silanes, such as aminosilane or 3-glycidoxypropyltrimethoxysilane, have been coated onto the glass surface, with the silane moiety attached to the glass and the reactive moiety cross-linked to probe molecules. Other approaches have utilized slides coated with reactive substrates with functional aldehyde, carboxyl, epoxy, or amine groups that can form a covalent bond with the probe molecules, affixing them permanently to the glass surface.

Although these methods work moderately well for small probe molecules, they tend to work poorly for larger probe molecules (e.g., antibodies) where functionality (binding) may depend on probe orientation, flexibility and degree of cross-linking. Covalent attachment methods also tend to bind very little material to the matrix surface. Consequently, probe concentration is low and signal detection is difficult. Because relatively little probe is available on the surface of the 2D array, such systems show a low signal-to-noise ratio for a positive binding reaction between probe and target.

Protein or peptide target molecules are often detected using antibodies as capture molecules. Two-dimensional arrays used in clinical diagnostics or proteomics frequently utilize antibodies as probes for protein or peptide target molecules. Although antibodies tend to be highly specific for their target antigens, they are not easily attached to glass or plastic surfaces with cross-linking agents and standard methods. This is because of the limited amount of material that can be affixed to the matrix with known chemistries, resulting in weak signals generated upon target binding. Another problem is that antibody specific binding cannot be maintained without adequate hydration and support in the matrix. Thus, long-term stability of antibody-coupled solid matrix arrays tends to be limited, with inconsistent results obtained depending on the age of the array.

Attempts have been made to solve this problem by creating an environment that stabilizes the protein and preserves its functional probe features. For example, Prolinx Inc. (Bothell, Wash.) has developed a chemical affinity system using standard glass slides with a polymer brush format affixed to their surface. The system relies upon the interaction between two synthetic small molecules that form a stable complex, phenyldiboronic acid (PDBA) and salicylhydroxamic acid (SHA). (E.g., Stolowitz et al., Bioconjugate Chem. 12:229-239, 2001.) PDBA is first conjugated with protein probes. The conjugated probes then link to SHA attached to the polymer brush to form a 3D functional array. This method is limited by the amount of antibody that can be bound to the surface. More importantly, the target antigen must be sufficiently small to diffuse through the brush border in order to react with antibodies affixed to the matrix. Such methods are not suitable for identifying and/or quantifying larger targets, such as whole cells or bacteria.

Opaque Slides

Methods to stabilize and increase the amount of probe attached to matrix arrays are highly desirable. Such methods generally lead to opaque slides, since the matrix materials used to increase probe binding and preserve stability typically involve non-translucent gels, hydro-gels, agars, and other materials coated on the glass surface. Proteins attached to such opaque matrix materials are stabilized by hydrophobic and electrostatic interactions in a three-dimensional array.

Most scanners in current use for genomic and proteomic microarrays read the slides from the same side as the bound probe and target molecules, using opaque matrix arrays. Opaque matrix-coating materials used to produce microarrays include nylon, PVDF (polyvinylidene fluoride) and nitrocellulose. Nitrocellulose, a traditional polymer substrate in use for more than 50 years, is a substrate with very attractive properties for microarray applications. (E.g., Tonkinson and Stillman, Frontiers in Bioscience 7:c1-12, 2002.)

Opaque nitrocellulose has been extensively used to immobilize proteins and nucleic acids for biomolecular analysis. Nitrocellulose immobilizes molecules of interest in near quantitative fashion and allows for short and long term storage. Nitrocellulose also allows for solution phase target species to efficiently bind to immobilized capture molecules. Diagnostic devices using ELISA methods have employed nitrocellulose membranes with a lateral flow process to bind capture reagents to the membrane (Jones, IVD Technology, 5(2):32, 1999).

Traditional opaque membrane materials have a number of attractive features. They are inexpensive to construct, bind more than 100 times the amount of protein that can be bound by linker coated glass slides, and are generally easy to work with. This is particularly true for opaque nitrocellulose membranes, which have a long history of use.

Nitrocellulose is normally produced in a microporous form that may be applied to the surface of glass slides to form an opaque surface. Probes may then be attached to the opaque nitrocellulose membranes in microarrays, using standard nitrocellulose binding methods. Such slides have been used with radioactive, fluorescent and chemiluminescent detection systems (e.g., Brush, The Scientist 14[9]:21, 2000).

Traditional nitrocellulose membranes are also very brittle in the absence of a supporting structure or foundation, leading to frequent cracking or fragmentation. For this reason, opaque nitrocellulose has been used in a microporous form bound to plastic sheets. Such sheets are always opaque, due to the microporous form, and require a supporting structure (e.g. acetate or cellulose) to avoid damage during handling.

Translucent Nitrocellulose Slides

The methods and compositions disclosed herein may be used to produce translucent surface coatings of colloidal nitrocellulose that retain the advantageous binding characteristics of opaque nitrocellulose membranes, while allowing for use of detectors arranged on the bottom (non-binding) side of the slide. The interaction between probe and target molecules may be observed directly on a translucent nitrocellulose solid matrix.

In some embodiments, translucent nitrocellulose matrix arrays may be used. In such embodiments, the translucent nitrocellulose matrix may be attached to one side of a glass or plastic slide (e.g., nylon). Probes may be attached to the nitrocellulose and the interaction between probe and target molecules observed through the slide with a sensor or camera.

The nitrocellulose material is totally translucent (i.e., transparent) if formed according to the disclosed methods. Light signals may thus be observed without scatter or interference from opaque materials. This allows a greater signal-to-noise ratio and ease of detection of target molecules, compared to opaque microporous nitrocellulose matrix arrays. Such opaque matrix arrays can obscure portions of the light or reaction indicator species (e.g., bioluminescent compound) produced upon binding of target molecules.

Nitrocellulose in the form of a colloid in an amyl acetate solvent has been used by electron microscopists to make castings for specimens. Colloidal nitrocellulose is formed by casting as an ultra-thin film on a water surface. The film may then be picked up on a transmission electron microscopy (TEM) grid and used as a support film for TEM specimens. Because the film must be very clean and uniform, great care is exercised in its production. Colloidal nitrocellulose is readily soluble in amyl acetate. The amyl acetate is water soluble and evaporates evenly to form uniform films. It is supplied as a 1% solution of very pure nitrocellulose.

High purity nitrocellulose in EM grade amyl acetate (Collodion) may be purchased from commercial sources. The amyl acetate is purified by refluxing over calcium oxide to remove all moisture. Soluble and suspended material is removed by slow distillation. The removal of all traces of moisture from the solvent permits the formation of very strong colloidal nitrocellulose films with virtually no holes.

In an exemplary embodiment, Collodion was obtained in bulk from Ernest F Fullam, Inc. (Latham, N.Y.) and used to manufacture high quality translucent nitrocellulose matrix arrays. An aliquot of 200 μl of 1% Collodion solution was pipetted onto the surface of freshly cleaned standard 25×75 mm glass slides. The Collodion was evenly spread to the edges of the glass slide surface in a dust free area. After drying for 2 hours at room temperature, the slides were heated for an additional hour or more at approximately 60° C. Dried slides were labeled and stored for production of microarrays.

When using a glass array surface, the edges of each slide were sealed with lacquer (e.g., nail polish) or other adhesive to prevent the ultra-thin nitrocellulose substrate from separating from the glass upon exposure to aqueous solutions. When colloidal nitrocellulose is applied to nylon slides, acetate film or other plastic surfaces, it requires no adhesive and binds avidly. Slides may be composed of almost any translucent material as long as the amyl acetate does not react with the surface to discolor it or alter its properties. Certain types of plastics become opaque when exposed to amyl acetate and are not suitable for use with that solvent system. In alternative embodiments, the colloidal nitrocellulose may be suspended in other volatile organic solvents besides amyl acetate before application to a nylon, glass or other translucent slide.

An advantage of the translucent nitrocellulose surface is that the progress of the probe binding reaction may be examined by looking through the translucent lower surface of the slide. This allows more effective probe binding to occur. The progress of the probe-target binding reaction may also be monitored in real time through the underside of the slide.

GRABBER™ Slides

Certain embodiments may concern devices and methods of use of translucent coated slides, e.g., GRABBER™ slides, prepared as disclosed above. GRABBER™ slides provide for an easy to use method for detecting and identifying one or more targets of interest in a solution assayed on the surface of a slide. Slides may be provided pre-spotted with capture probes (e.g., antibodies) affixed ready for immediate testing. Alternatively, users who want to prepare their own 2D-spotted array may in 1 hour prepare a manual array for testing.

In certain preferred embodiments, the method of detection utilizes a primary antibody capturing an antigen target to form an immunocomplex with a second antibody that is biotinylated or otherwise labeled. The first antibody probe is affixed to a translucent nitrocellulose-coated slide (GRABBER™) and during the entire procedure remains affixed to the surface of the slide.

The immunocomplex, if formed, may be detected using a well-known enzyme activated bioluminescent process. For example, horseradish peroxidase (HRP) catalyzes the breakdown of peroxide and produces an intense light if luminol is added to the solution during the breakdown process. Luminol forms an unstable free radical intermediate that results in the release of photons at 430 nm. By conjugating HRP to streptavidin (SA), the immunocomplex may be detected with an appropriate and sensitive photon detector. Biotin and streptavidin rapidly combine to form this light producing complex.

Typical protein (e.g., antibody) probes may be spotted with 1 to 5 nl fluid per spot at a concentration of 0.05 to 0.2 mg/ml. Where primary antibodies are attached to a slide, matrix array or other surface, secondary tagged antibody, such as biotinylated antibody, may be provided in a dilution buffer premixed at an optimized concentration for target detection with pre-spotted slides. The optimized concentration may be determined empirically by the user. Secondary antibodies may be diluted in dilution buffer to a concentration typically in the range of 5 to 30 μg/ml.

A TOAD™ (total optical assay device) may be provided with software to allow the user the means to read, interpret and record signals at the surface of a slide. An image file and report may be saved or printed for subsequent analysis and comparisons.

GRABBER™ slides may be coated with a translucent nitrocellulose substrate (see U.S. patent application Ser. No. 10/373,546, filed Feb. 24, 2003) that avidly and immediately binds proteins, carbohydrates and/or oligonucleotides, strongly affixing them to the coated surface while preserving the functionality and 3D structure of the bound molecule. No other chemical process is required to affix the probes to the slide surface, making the 2D array procedure simple and fast. A hydrophobic surface with a superior contact angle is well suited for compact robotically printed arrays.

The bioluminescent reaction is long lasting and the signal may be acquired over prolonged periods, allowing for extraordinary sensitivity in detection. Many targets may be simultaneously identified in a single sample. The proper primary probe antibody and biotinylated secondary antibody should strongly interact with the target for adequate detection. Preprinted slides and reagents provided are optimized to produce high quality detection. The skilled artisan will realize that translucent coated slides are merely one preferred alternative for detection of target binding and that other alternatives, such as the magnetic beads discussed below, may be used with an optical detection system.

Reconfigurable Microarrays

In certain embodiments, reconfigurable microarrays may be produced by using small linker molecules, such as aptamers or affibodies, bound to the surface of a solid matrix. Aptamers are oligonucleotides derived by an in vitro evolutionary process called SELEX (e.g., Brody and Gold, Molecular Biotechnology 74:5-13, 2000). Aptamers may be produced by known methods (e.g., U.S. patent Nos. U.S. Pat. Nos. 5,270,163; 5,567,588; 5,670,637; 5,696,249; 5,843,653) or obtained from commercial sources (e.g., Somalogic, Boulder, Colo.). Aptamers are relatively small molecules on the order of 7 to 50 kDa. Because they are small, stable and not as easily damaged as proteins, they may be bound in higher numbers to the surface of a solid matrix. This effectively amplifies the number of probe reactive sites on the surface of an array.

Affibody® ligands (U.S. Pat. No. 5,831,012) are highly specific affinity proteins that may be designed and used like aptamers. Affibodies may be produced or purchased from commercial sources (Affibody AB, Bromma, Sweden). Aptamers and affibodies may be used in combination with antibodies to increase the functional avidity of translucent or non-translucent solid matrices for probe molecule binding. Increased binding in turn results in an increased signal strength, greater signal-to-noise ratio, more reproducible target molecule detection and greater sensitivity of detection.

Reconfigurable microarrays may be used in combination with two antibodies and a capture probe. The capture probe may be an affibody, aptamer or any other probe capable of binding one of the antibodies. Both antibodies should selectively bind to a target cell, molecule or antigen.

The effectiveness of binding is increased if the capture probe binds to a portion of an antibody characteristic of the IgG class. Such probes would only require a small part of the antibody structure to be present in order to react and bind to an antibody-target complex. Larger targets, such as microbes or cells are covered with numerous antigens that may form very large complexes with antibodies. However, truncated IgG antibody fragments could interact with such large targets and still bind to an aptamer or affibody probe on the slide surface.

In certain embodiments, two antibodies are allowed to bind to the target in solution. Once target-antibody complexes are formed, the complex may be exposed to aptamer or affibody probe molecules on a reconfigurable matrix array. The probes may bind to a first antibody, while the second antibody may be conjugated to a bioluminescent tag or other marker. The tagged complex may then be detected on the surface of the matrix array, using optical detection or any other known detection method.

For example, an aptamer may be tailored to specifically bind to the Fc portion of mouse IgG with high affinity. Samples containing target molecules of interest may be allowed to interact in solution with a mouse antibody specific for an antigen of interest. The sample is mixed with a different biotinylated or otherwise tagged second (non-mouse) antibody that binds to a different epitope on the same antigen. The target antigen bound to the first and second antibodies is exposed to the aptamer microarray. The anti-mouse aptamer affixes the complex to the solid matrix. After extensive washing to remove unbound tagged antibodies, the complex containing tagged antibody that is attached to the matrix array surface is detected.

The skilled artisan will realize that many variations on this scheme may be used. For example, in alternative embodiments, a first antibody may be used in conjunction with multiple tagged second antibodies, each of which binds to a different epitope of the target molecule. This may occur, for example, where the available second antibodies are polyclonal antibodies. Alternatively, use of more than one second antibody with affinity for the same antigen may improve the sensitivity of detection. In another alternative, one second antibody may bind to a class of targets (for example, all E. coli bacteria) while a second antibody binds to a specific subclass (e.g., E. coli strain O157:H7).

In a non-limiting example, to detect Listeria monocytogenes, an IgG mouse anti-Listeria m. antibody may be incubated with a food sample of interest at an appropriate concentration (typically 1 to 50 μg/ml). A rabbit (or other non-mouse) biotinylated secondary anti-Listeria m. antibody (1 to 50 μg/ml) may be added and incubated for 5 to 30 minutes. The sample with both antibodies may then be applied to the array containing anti-mouse IgG aptamers. After a short interval (approximately 15 minutes) the array may be washed so that only mouse IgG and rabbit biotinylated antibody complexed with Listeria m. is retained on the array. A solution of HRP-conjugated strepavidin or other indicator applied to the surface may then reveal the presence or absence of an anti-Listeria m. antibody complex affixed to the surface.

Magnetic Beads

In certain embodiments, the probes, antigens or other ligands of interest may be attached to magnetic beads for separation and/or detection of target binding. Additional details of protocols for use with magnetic beads are disclosed in the Examples section below. In preferred embodiments, the probes, antigens or other ligands attached to the beads may be proteins or peptides, such as antibodies, antibody fragments, antibody binding proteins (e.g., protein A) or target antigens. Processes for the coupling of molecules to magnetic beads or a magnetite substrate are well known in the art, i.e. U.S. Pat. Nos. 4,695,393, 3,970,518, 4,230,685, and 4,677,055. Attachment may be either covalent or non-covalent. A number of potential chemical cross-linking agents are well known in the art, including EDC, dinitrobenzene, bisimidates, N-hydroxysuccinimide ester of suberic acid, dimethyl-3,3′-dithio-bispropionimidate, 4-(bromoaminoethyl)-2-nitrophenylazide, disuccinimidyl tartarate and azidoglyoxal.

It is envisioned that the magnetic particles employed may come in a variety of sizes. While large magnetic particles (mean diameter in solution greater than 10 μm) can respond to weak magnetic fields and magnetic field gradients, they tend to settle rapidly, limiting their usefulness for reactions requiring homogeneous conditions. Large particles also have a more limited surface area per weight than smaller particles, so that less material can be coupled to them. In preferred embodiments, the magnetic beads are less than 10 μm in diameter.

Ferromagnetic materials in general become permanently magnetized in response to magnetic fields. Materials termed “superparamagnetic” experience a force in a magnetic field gradient, but do not become permanently magnetized. Crystals of magnetic iron oxides may be either ferromagnetic or superparamagnetic, depending on the size of the crystals. Superparamagnetic oxides of iron generally result when the crystal is less than about 300 angstroms (Å) in diameter; larger crystals generally have a ferromagnetic character.

Dispersible magnetic iron oxide particles reportedly having 300 Å diameters and surface amine groups are prepared by base precipitation of ferrous chloride and ferric chloride (Fe²⁺/Fe³⁺=1) in the presence of polyethylene imine, according to U.S. Pat. No. 4,267,234. These particles are exposed to a magnetic field three times during preparation and are described as redispersible. The magnetic particles are mixed with a glutaraldehyde suspension polymerization system to form magnetic polyglutaraldehyde microspheres with reported diameters of 0.1 μm. Polyglutaraldehyde microspheres have conjugated aldehyde groups on the surface which can form bonds to amino containing molecules such as proteins.

While a variety of particle sizes are envisioned to be applicable in the disclosed method, in a preferred embodiment, particles are between about 0.1 and about 1.5 μm diameter. Particles with mean diameters in this range can be produced with a surface area as high as about 100 to 150 m²/gm, which provides a high capacity for bioaffinity adsorbent coupling. Magnetic particles of this size range overcome the rapid settling problems of larger particles, but obviate the need for large magnets to generate the magnetic fields and magnetic field gradients required to separate smaller particles. Magnets used to effect separations of the magnetic particles need only generate magnetic fields between about 100 and about 1000 Oersteds. Such fields can be obtained with permanent magnets that are preferably smaller than the container which holds the dispersion of magnetic particles and thus may be suitable for benchtop use. Although ferromagnetic particles may be useful in certain applications of the invention, particles with superparamagnetic behavior are usually preferred since superparamagnetic particles do not exhibit the magnetic aggregation associated with ferromagnetic particles and permit redispersion and reuse. In a preferred embodiments, a pair of spherical magnets may be juxtaposed to a container, such as a 1.5 ml Eppendorf tube, and used to collect magnetic beads on the side of the tube. The use of spherical magnets provides a more intense localized magnetic field, which facilitates separation of magnetic beads from solution.

The method for preparing the magnetic particles may comprise precipitating metal salts in base to form fine magnetic metal oxide crystals, redispersing and washing the crystals in water and in an electrolyte. Magnetic separations may be used to collect the crystals between washes if the crystals are superparamagnetic. The crystals may then be coated with a material capable of adsorptively or covalently bonding to the metal oxide and bearing functional groups for coupling with proteins or other ligands. Commercial sources of magnetic beads are also known in the art and may be used, for example Dynal Biotech (Brown Deer, Wis., USA).

Image Sensing and Data Analysis

In various embodiments, positive assay results may be detected by, for example, analysis of an optical signal emitted by a chemiluminescent target-ligand complex, as exemplified in Example 1. Various methods and apparatus for optical signal detection and analysis are known in the art and may be used in the claimed methods. For example, complementary metal oxide semiconductor (CMOS) image sensors are used in digital cameras and are increasingly found in a variety of analytic instruments. CMOS image sensors are improving in quality and are challenging and replacing charge-coupled device (CCD) imagers for detecting low level spectral images.

Most modern light detectors are designed to capture a spectral signal by presenting a two-dimensional array of sensitive photodiodes towards a target. The photodiodes are designed to produce current when exposed to light, and the resulting current may be analyzed in various ways. Modern sensors convert the analog photodiode signal to a digital signal format that may then be stored and processed for later analysis. High-resolution digital pictures may be produced pixel by pixel with an appropriate source of light, an optical system, an image sensor, and a computer. Using such a system, photographic pictures may be obtained in either monochromatic or color formats.

However, a photodiode will produce an analog output signal that correlates with the energy striking the photodiode array only in special circumstances, such as when the target is illuminated by monochromatic light at a particular wavelength. Even though the output signal of a photodiode is essentially linear with respect to the illumination applied to the photodiode, the signal value for a pixel does not generally correlate accurately with the photon flux. This is because the quantum efficiency (QE) for converting the photon flux to a photodiode electrical energy varies with certain factors. In addition, in most cases more than a single wavelength of light will strike a photodiode.

Every photodiode has a certain QE value that will vary with factors such as wavelength and temperature. Photon flux represents the electromagnetic energy striking the surface of a two-dimensional array, and the QE represents the capability of the photodiode to convert that energy into electrical energy. QE is usually expressed as a percentage of the energy flux, equaling some percentage less than 100 percent. Because QE varies greatly with the wavelength of light illuminating a photodiode, comparisons of a signal at one wavelength to that at another are difficult to interpret unless the QE factors are known for all wavelengths that apply. Further, most image sensors are designed by manufacturers to produce images that approximate the equivalent of what would be seen on a film or by the human eye. Manufacturers are interested in reproducing “life-like” pictures and colors. Manufacturers may provide access to the raw digital information for every pixel, but image sensors generally process that information before it is available for analysis to better render the “life-like” colors and intensities that represent human visual expectations. For these reasons, the data produced by an image sensor generally does not directly relate to the photon flux that impinges upon the photodiode array of the sensor. This factor limits the usefulness of image sensors for analytic purposes.

Image Sensor Operation

Investigators using a photodiode detector may incorrectly assume that the digital data acquired from the detector correlates with the photon flux striking the detector because increases in the intensity of the signal out will generally directly correlate with increases in signal input in a particular wavelength or band-width. Because photodiodes are very linear in output, increases in the photon flux at different wavelengths over the photodiode surface will, over the photodiode's dynamic range, produce a linear output signal. However, the output data will not correlate with the photon flux if the QE is variable over the range of wavelengths that are striking the photodiode. For a particular number of photons striking a photodiode over a time period, a larger current will be produced by the photodiode at a first wavelength than a second wavelength if the QE for the photodiode is higher for the first wavelength than the second wavelength.

Restricting a light source for analysis to a narrow band by filtering or by using a laser light source generally will not resolve accuracy issues. Emission spectra that are evaluated using image sensors may be very broad even if the excitation source has a narrow wavelength range. For this reason, the shape of the QE curve for a photodiode should be carefully considered in evaluating output data from an image sensor.

With regard to the choice of image sensors, CMOS imager sensors are fundamentally different from charge-coupled devices and are increasingly used in microscopy and diagnostic instruments because they are cheaper to build and require considerably less power to operate. CCD cameras are no longer clearly superior in low intensity light situations, which had been true in the past. CMOS images now rival traditional color imaging methods on film and are easily manipulated.

The images may be transferred from one processor to another as a digital file in a variety of formats preserving the arrayed data pixel address. Manufacturers have devoted considerable energy reproducing “life-like” color image sensors using various color filters and interpolation methods to enhance a digital image rendering colors very close to the human eye experience. However, the pleasing “life-like” color pictures obtained with a color CMOS image sensor are not as useful for analytic procedures. Similar issues exist with monochromatic images unless the image is produced by light at single wavelength, which is rarely true.

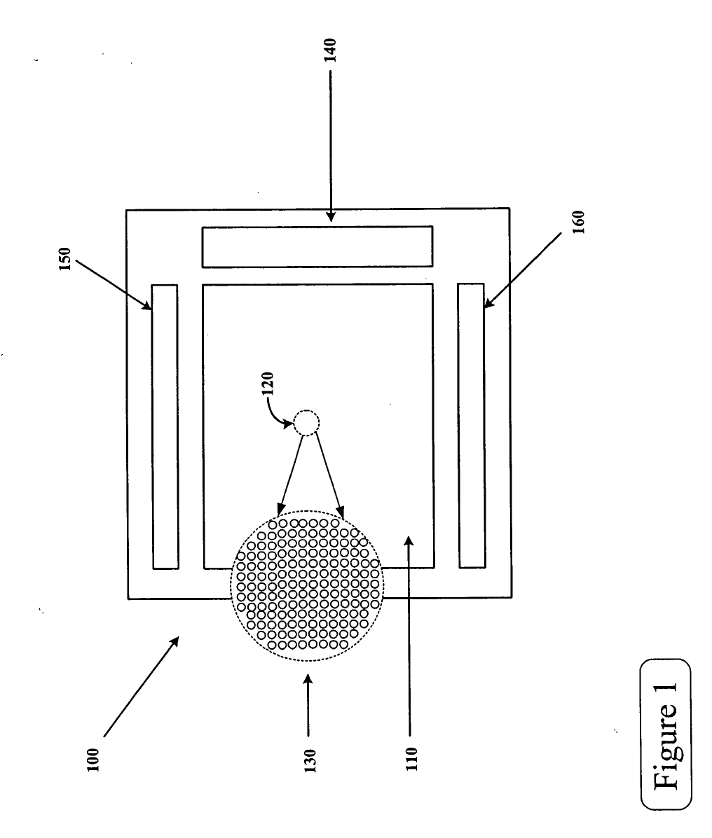

FIG. 1 is a simplified illustration of an example of a color CMOS image sensor that may be utilized. Not all components and features of CMOS imagers are illustrated. FIG. 1 and the remaining figures in this section are for illustration and are not necessarily drawn to scale. In FIG. 1, an image sensor 100 includes an imaging array 110. The imaging array is comprised of a large number of pixels arranged in a two-dimensional array. As shown in the magnified pixel array 120 of a particular area of the array 110, there is a filter associated with each of the pixels in the imaging array 110. The image sensor 100 will also generally contain electronics relating to the processing and transmission of signals generated by the imaging array 110, including analog signal processing 140, analog to digital conversion 150, and digital logic 160.

The photodiode array in a CMOS color image sensor is blanketed by an ordered thin layer of polymeric filters, such as in a conventional Bayer RGB (red-green-blue) two-dimensional array. Each filter is sized to fit over an individual photodiode in a sequential (Bayer) pattern to capture color information from a broad bandwidth of incident illumination. In an RGB array, a heavy emphasis is placed on the green filters to address the human visual maximal response at 550 nm. There are 2 green filters for every red and for every blue filter. However, even though the human eye is more attuned to the green 550 nm region, yellow is generally a better choice with regards to QE factor.

CMOS image sensors and the integrated circuits that define the active pixel array are inherently monochromatic (black and white) devices that respond only to the total number of electrons striking the photodiodes, not to the color of light. Color is detected either by passing the light through a sequential series of filters (such as red, green, and blue filters), or with miniature transparent polymeric thin-film filters that are deposited over the pixel array.

Active pixel sensor (APS) technology is the most popular design for CMOS image detectors. In addition to a photodiode, each pixel (or imaging element) includes a triad of transistors on its surface that convert accumulated electron charge to a measurable voltage, reset the photodiode, and transfer voltage to a vertical column bus. The photodiode thus occupies only a fraction of the pixel area. The photodiode area encompasses an area equal to 30 to 80 percent of the total pixel area for most CMOS sensors. This area occupied by the photodiode is the area that absorbs photons, while the other parts of the pixel are relatively opaque, blocking, reflecting, or absorbing light. The photodiode area or window is referred to as the “aperture” or “fill factor” of the pixel or image sensor. A small aperture or fill factor results in a significant loss of sensitivity and a corresponding reduction in the signal to noise ratio and leads to a reduction in the dynamic range of the sensor.

CMOS image sensors can be utilized to produce pictures based upon the signals produced when photons strike the photodiode surface associated with each pixel in an active pixel sensor array. The pixel signals are processed to form the total picture either in monochrome (black and white) or color. Monochrome CMOS imager sensors do not have color filters over the photodiode portion of the pixel. However, color CMOS imagers, even with standard Bayer pattern filters, generally are more sensitive than monochromatic CMOS imagers. While it may appear that inherently monochromatic CMOS photodiode without filters would be more sensitive because some light passing through a filter is absorbed and never reaches the photodiode in a color filtered photodiode, this is not generally true. This assumption does not fully take into account the effect a filter has on the QE for a photodiode, which might enhance certain signals, and ignores the advantages provided by microlenses in color photodiode architecture, which are described below. Monochromatic CMOS image sensors do not have a color filter and they do not normally have microlenses over each pixel. These are important factors that make monochromatic imagers less attractive than color CMOS image sensors with regard to imager sensitivity.

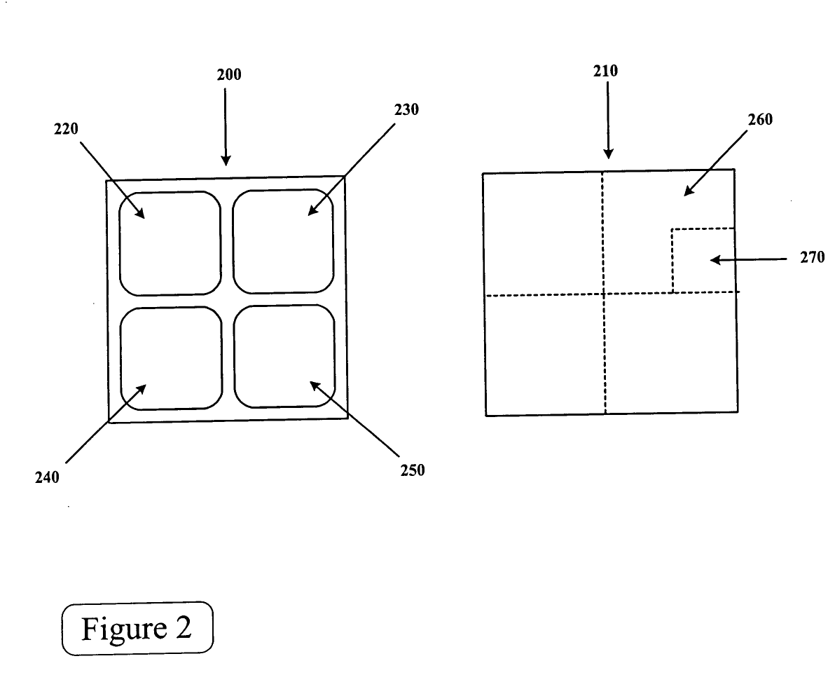

FIG. 2 illustrates a small section of an imaging array of a color image sensor that may be utilized. The illustrated section repeats throughout the imaging array. The section 200 is comprised of four pixels, each having a filter. The filters 220-250 have colors based on the choices made for the array. Below the filters 210 illustrates the structure of the individual pixels. For each pixel, such as pixel 260, there is a portion that comprises the photodiode 270, the area of the photodiode being only a fraction of the total area. The image sensor will detect only the portion of the light falling on the photodiode area.

In color imagers used in analysis, one possible approach would be to construct imagers by carefully selecting filters and photodiodes to produce QE factors for a given bandwidth that is approximately constant. By combining an appropriate number of photodiodes in an array with chosen filters, the measurement of light energy would be more accurate. The filters chosen could assist in leveling and improving upon the QE factors. However, in practice the filters and photodiodes are chosen for other purposes, with a goal of producing the most visually pleasing images. In order to improve upon the quantum efficiency and spectral response, several CMOS manufacturers use color filter arrays based on the primary subtractive colors, cyan, magenta, and yellow (CMY), instead of the standard additive primaries red, green, and blue (RGB). CMOS image manufacturers generally use either Bayer RGB or Bayer CMY patterns that have been selected for photographic imaging.

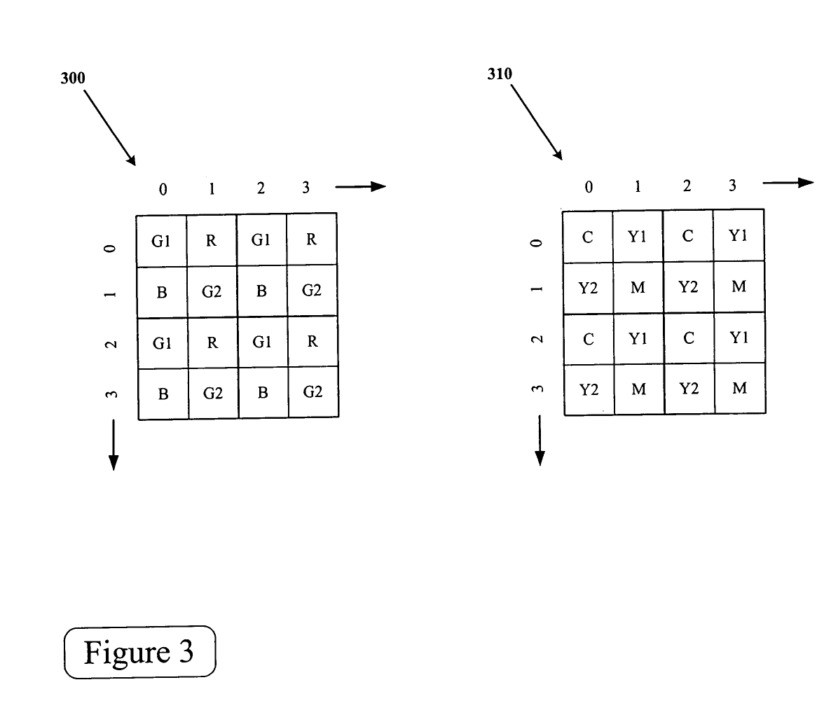

FIG. 3 is an illustration of RGB and CMY filters. For these filters, an imaging array is divided into small arrays of filters, with each such array of filters having the same filter pattern. An RBG filter array 300 contains two-by-two arrays of filters, with each array containing a red filter and a blue filter for two diagonal pixels and two green filters for the remaining two diagonal pixels. A CMY filter array 310 also contains two-by-two arrays of filters, with each array containing a cyan filter and a magenta filter for two diagonal pixels and two yellow filters for the remaining two diagonal pixels. While the illustrated filters are the most common filter arrangements, many other filter colors and patterns are possible, and any filter pattern may be used. Certain other alternative filter patterns that provide benefits in certain wavelength ranges are shown in Table 3.

In contrast to monochrome image sensors, color CMOS image sensors also contain microlenses that effectively direct photons to the photodiode aperture. The bubble lens, generally including an anti-reflective coating, can effectively increase the surface area of a photodiode by a significant amount, approximately 60 percent in certain applications. The microlenses substantially increase the effective fill factor and may more than compensate for filters that cut down on the total light that can reach the photodiode.

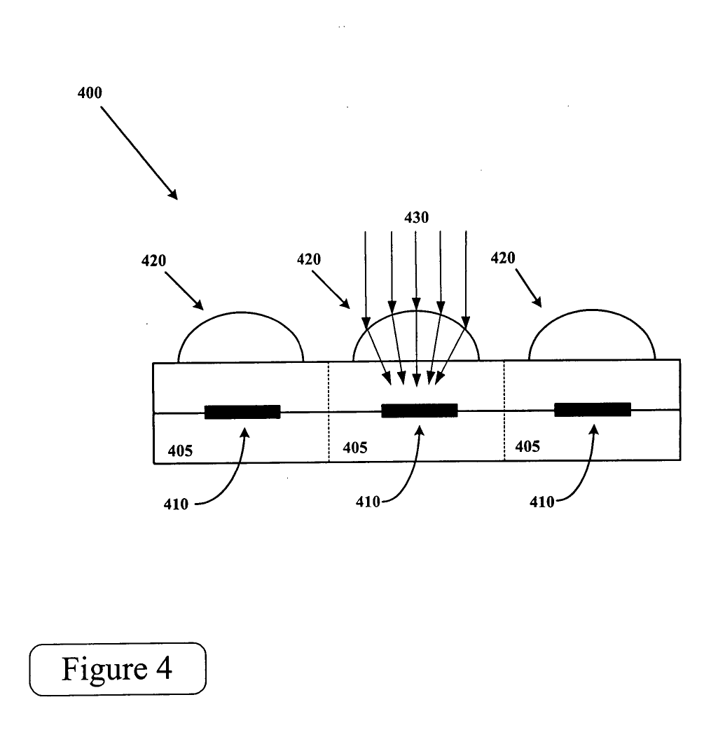

FIG. 4 is a simplified illustration of microlenses in an image sensor. Within the image sensor, there are multiple pixels 405. Each pixel contains an active portion 410, with the active portion including only a portion of the pixel area. In order to compensate in part for the light energy that would not normally strike the active portions 410, each of the pixels 405 has an associated microlens 420. The function of each microlens 420 is to focus more light energy on the active portion 410 and thus to allow measurement of a larger percentage of the incident photon flux. For example, light 430 strikes a microlens 420 and is focused on the active portion 405 of the pixel 410.

Image Sensor Optimization

Three primary mechanisms that reduce or hamper photon collection by the photosensitive area of an image sensor are absorption, reflection, and transmission. These factors are wavelength-dependent in nature, and define in part the quantum efficiency (QE) of the image sensor. For example, reflection and transmission of incident light occurs as a function of wavelength, with a high percentage of shorter wavelengths below 400 nm being reflected. Shorter wavelengths are absorbed in the first few microns of the photosensitive region but the longest wavelengths exceeding 650 nm often pass through the photosensitive region.

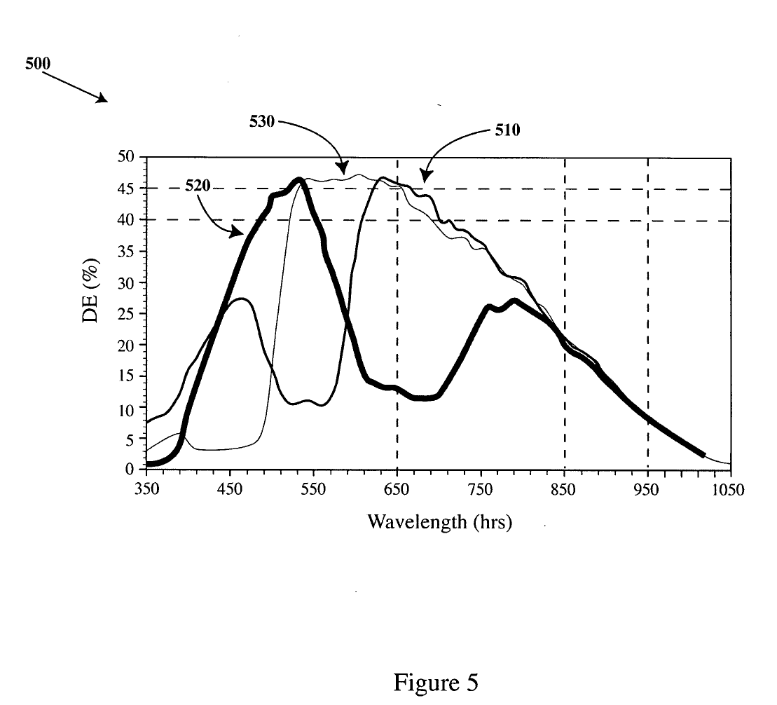

FIG. 5 illustrates a typical quantum efficiency spectral response for an image sensor. For FIG. 5, a Bayer CMY filter is evaluated. The spectral response 500 illustrates the quantum efficiency of the image sensor for various wavelengths of light incident on the image sensor. There is an individual response curve for a pixel with a magenta filter 510, a pixel with a cyan filter 520, and a pixel with a yellow filter 530. Each curve has peaks and valleys at different wavelengths of incident light.

By examining the QE wavelength dependence curves for each filter type used in an image sensor, the output signal proportional to the photon flux can be determined for any wavelength or interval of interest, including those pixels for a monochromatic image sensor. In many cases every pixel in an array is essentially identical to its neighbor except for the kind of filter (CMY, RGB, other pattern, or no filter). The effect of a filter is either to increase or to reduce the photodiode energy output for a given photon flux. The effect on the signal is wavelength dependent. The QE is the variable in the output signal that should be factored out of the equation if fair comparisons are to be made across the imaging array for each and every pixel.

In a CMOS imager, the pixel signal is obtained for each pixel as raw data after the analog to digital converter transforms the value for a set time interval. If QE is expressed as a fraction, the pixel signal is directly proportional to the product of the QE and Photon Flux:
Pixel Signal=Constant×QE×Photon Flux

If raw data can be normalized according to the appropriate QE, digital values can be created that may be used for more accurate subsequent analysis. The pixel value for a color CMOS image sensor may be obtained before the on chip conversion occurs and the value normalized by multiplying each signal value for a particular color filter by the inverse QE. For a relatively narrow bandwidth, the QE may be treated as a constant depending upon the wavelength and filter type used. In one example, the Bayer CMY pattern over the range 550 to 650 nm for a Kodak 1310 color CMOS image sensor provides a QE of approximately 46 percent, and then drops linearly from 650 nm to 5 percent at 990 nm, approximately 0.6 percent every 5 nm. In addition, the Magenta and Yellow filters are very similar over the range from 630 nm to 990 nm.

For a particular example with a CMY pattern Kodak 1310 color image sensor at 670 nm, the QE values are as shown in Table 4. A pixel with a yellow filter would have its digital raw data multiplied by 2.38, a magenta filter pixel by 2.27, and a cyan filter pixel by 7.69. In this embodiment, the signal for every pixel is effectively transformed to a numeric value that is directly proportional to the actual photon flux. It is noted that Table 4 only contains the QE for the image sensor when light of a particular wavelength (670 nm) strikes the image sensor. The QE for any other wavelength of light will vary, as shown in FIG. 5.

QE Factor Correction

Corrections to account for differences in QE may be made based upon known QE factors for a particular filter type and wavelength. (For example, see the data contained in Table 3 and Table 4.) However, an image sensor may also be utilized to automatically correct for differences in pixel QE. Each area of a sensor array, such as each filter quadrant, may be normalized to render every pixel in the quadrant optimally tuned for photon flux in real time. No corrections are made if the pixels and filters are all of the same type, as, for example, the YYYY, MMMM, and CCCC filter patterns shown in Table 3. A correction may be made if there are two or more filter types in the array (e.g., filter patterns such as YYYC, RGB Bayer, or modified CMY Bayer). A method of auto correcting for QE may be used for any combination of two or more filter and photodiode types and such method corrects to normalize the 4 pixels in a quadrant so that each pixel produces an equivalent output signal.

If the pixels are tightly packed in a quadrant relative to the change in photon flux over a given region of the array, then it can be assumed that the same number of photons are striking each pixel in the quadrant at any given moment. With the currently available high-resolution sensors, and with anticipated future improvements in resolution, the assumption that neighboring pixels in any given quadrant experience identical photon flux is appropriate. Using this assumption, each of the pixels in the quadrant should produce the same output. Accordingly, auto correction may be used to make adjacent neighbors in each filter quadrant identical. The most sensitive pixel type in a quadrant may be used to factor out QE and wavelength differences, which simplifies the problem of correcting for wavelength and bandwidth dependence. In one example, once a background correction factor is determined for the most sensitive pixel in a quadrant, the same background correction factor may be assumed for each of the other pixels in the same quadrant. Auto correction also reduces or eliminates problems related to temperature variations for different filter and photodiode types. An automated method for threshold and normalized luminosity assignments is discussed in the following section.

In another example, an array of an image sensor comprises multiple filter quadrants. Two or more filters are used in each quadrant of 4 pixels. In each quadrant of 4 pixels, the average analog to digital converted signal output for each filter and photodiode type is determined. If, for example, there are 3 yellow filtered pixels and I cyan filtered pixel in the quadrant, the average for the 3 yellow pixels is determined first. The output value for the yellow pixels is then compared to the value for the cyan pixel to determine which output is numerically greater. Under the embodiment, there is an assumption that all 4 pixels receive equivalent photon flux. The highest output value is assigned to all four pixels in the quadrant. The next quadrant in the array may then be corrected in the same manner, with the process continuing until the entire array has been assigned corrected output values to correlate with photon flux.

The process of auto correction is repeated over time as an image sensor is used to record images. In yet another example, the wavelength of light received by an image sensor may change from a first wavelength to a second wavelength. A first type of filter may provide the highest QE for the first wavelength, while a second type of filter provides the highest QE for the second wavelength. The change in wavelength may be included in the calculation process and therefore auto correction for changing light can be made in real time.

It is not necessary to know in advance the QE for each filter type to auto correct for QE differences. Auto correcting the sensor based on the photon flux at the time an image is obtained optimizes the photo image to correlate with photon flux. This method of correcting the signal removes temperature and wavelength dependence differences for different filter types and can be implemented using software. Such method thus automatically corrects for a broad band signal impinging upon an image sensor. In yet another example, the digital signals produced by an image sensor auto corrected for photon flux may be rendered to a gray scale image for subsequent visualization in a monochromatic representation.

Image Sensor Calibration

The optimization of an image sensor may include a calibration step. The calibration may be accomplished by illuminating the color filters and photodiodes with light of known wavelength and intensity. For a color CMOS imager, the raw data for each filter is obtained and compared to expected values. From the resulting comparison, the QE and the multiplier (normalization factor) that is required to obtain the equivalent output signal for each color filter used for each and every pixel in the array may be obtained.

Optimized signals obtained using QE factor conversions can more accurately relate the signal to the photon flux, and therefore more precisely characterize events, such as the optical events related to excitation-emission spectra or absorption phenomena in a chemical reaction. Both sensitivity and accuracy are enhanced by properly converting the signal to account for QE factors. Using a standard CMOS imager (such as a Kodak 1310 color CMOS image sensor,) raw data produced may be processed for signal optimization. The signal is converted to a numeric value that correlates with the photon flux incident upon the imager. This process can be applied to either a color or monochromatic CMOS imager sensor to render the signal proportional to photon flux.

Data processed may be rendered for visualization, such as via a gray scale standard (0 to 255 monochromatic) to producing a black and white image that correlates with the actual photon flux. The visual image of the data is superficially equivalent to a gray scale monochromatic image sensor, but for an equivalent luminance will be more intense than a image produced by a monochromatic non-transformed CMOS counterpart because color CMOS chips are generally more sensitive than monochrome chips. A color image sensor generally provides a better signal and is more sensitive than a monochromatic imager because the pixel photodiode filters improve upon the QE for the photodiode. The filtering of light by a color image sensor may be corrected using the QE factors to convert the signal to a number that is directly proportional to the photon flux. Further, advantage then is taken of the color filter's microlens effectively amplifying the aperture for the photodiode.

Illustrations of Processes

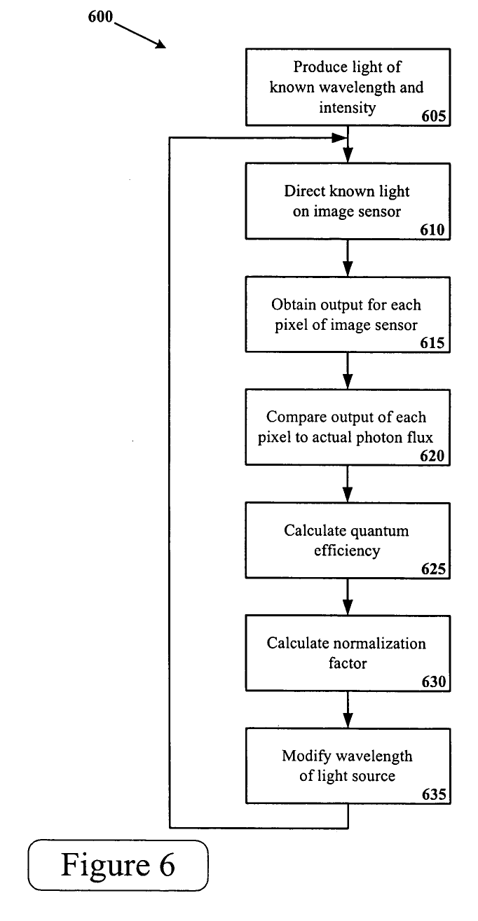

FIG. 6 illustrates a non-limiting example of a process for calibration for optimization of an image sensor. In the calibration process 600, a light of a known wavelength and intensity is produced 605. With a known intensity, the photon flux on each pixel is known, which would be the output if the QE of a pixel were 100 percent. The known light is directed on an image sensor 610. The output of each pixel of the image sensor is obtained 615. The output of the image sensor then can be compared with the actual photon flux 620. Using the comparison, the quantum efficiency of the pixel can be calculated 625, and then a normalization factor is calculated based upon the quantum efficiency 630. For a color CMOS image sensor, the comparison and calculation can be done for each filter color, resulting in a normalization factor for each filter color. In other embodiments, the comparison and calculation can be made for each pixel of an image sensor or for sectors of pixels of an image sensor, resulting in normalization factors that apply for certain portions of the image sensor. As the normalization factor varies for each wavelength of light that strikes the image sensor, the wavelength of the known light is varied 635 and the process repeats for each needed wavelength.

In certain embodiments, the image sensor may be a color sensor containing an array of pixels, with each pixel having a filter. The filters may be arranged in quadrants, with each quadrant having a particular filter pattern. The outputs of each of the pixels within a first quadrant of the array may be obtained. The average output of for each filter type in the quadrant is then determined. In one example, if a filter quadrant is CMY pattern containing a cyan filter, a magenta filter, and two yellow filters, the cyan output, the magenta output, and the average of the two yellow outputs are determined. The outputs are then compared and the highest output is determined. The highest output is then assigned to each pixel in the quadrant. For example, if the average yellow output is the highest output for the CMY quadrant, indicating that, under the particular conditions, the yellow filter has the highest QE factor, then the average yellow output is assigned to each of the pixels in the quadrant. If there are more quadrants in the array, the output of the next quadrant is obtained and the process continues. Once the final quadrant has been corrected the process is completed and the corrected output for the array is available. The process can then be repeated over time to allow real time QE factor correction for the image sensor.

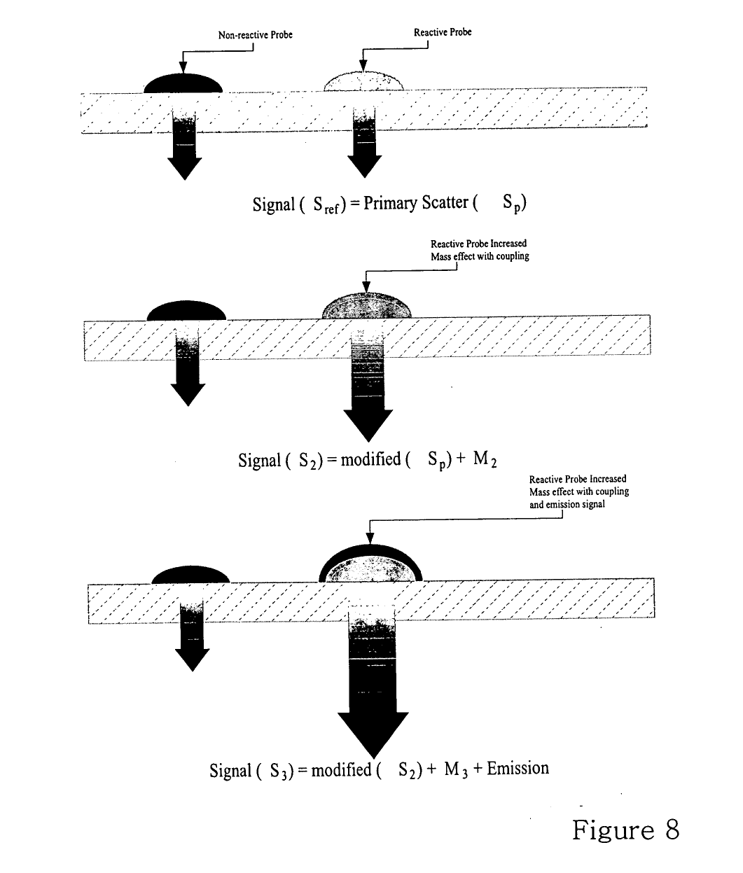

FIG. 8 is a flowchart illustrating an exemplary process for optimization of an image sensor. In the optimization process 700, an image of an event is captured with an image sensor 705. Under one embodiment of the invention, the image sensor is a color CMOS image sensor utilizing a filter pattern such as a Bayer RGB or CMY pattern. The raw data for the image of the event is obtained from the image sensor 710. The raw data is non-optimized data that, due to the nature of the image sensor, will generally vary greatly from the actual photon flux that struck the image sensor. As the normalization factor depends on the wavelength of light, the wavelength is determined 715. The appropriate normalization factor is determined for each pixel 720 based upon the wavelength of light. For one embodiment utilizing a color CMOS image sensor, a normalization factor for each lens color is used in normalization. Under other embodiments, the normalization factors may vary based on other factors. The raw data is then converted using the appropriate normalization factors for the pixels of the image sensor 725, thus producing an optimized data set that approximates the actual photon flux for the captured image of the event. Under an embodiment of the invention, an image may be produced using the converted data 730.

Fluorescence Detection

Fluorophores are frequently used to detect the presence or absence of a coupled reaction on a glass surface. Fluorescence detectors measure the intensity of the evanescent wave produced when a fluorophore is excited with a laser or other light source. Typically the laser is used to excite the fluorophore at its absorption peak and the detector is tuned to read the emission signal at a longer emission wavelength, which is characteristic of that particular fluorophore. The shift in wavelength between absorption and emission is referred to as the Stokes shift. Most fluorescence detection methods use fluorophores with a large Stokes shift so that the emission and absorption curves are well separated. With fluorophores that have a small Stokes shift, it is necessary to excite at a shorter wavelength than the optimal peak absorption maximum because of overlap between the emission and absorption curves. The signal emission intensity is reduced and the sensitivity for detecting target molecules is decreased. The need for a large Stokes shift also limits the choices of fluorophores that can be used.

Because the curves for absorption and emission are frequently very near to one another, accurate reading of the emission signal may be complicated. If the distance between the emission and absorption curves is small, it is difficult to separate the light from an emission spectrum from that of the absorption signal. Lasers with a narrow band at the absorption peak are frequently used with filters to cut out all light up to a certain critical point just below the emission spectral curve. By selecting an appropriate long pass filter, band pass filter, or combination of long pass and band pass filters, the emission signal can be observed in a narrow window, eliminating much of the interference from the excitatory light source. Interference from the excitatory light source is also avoided by aligning the detector and apparatus so that the emission signal can be read at a large incident angle to the excitation beam. Although filters eliminate most of the signal from the excitatory light source, they also cut out a significant portion of the evanescent (emitted) signal. Most band pass filters cut out as much as 40 to 50% of the emission signal. Long pass filters may cut an additional 10% of the emission signal.

Fluorescent detection is used in a number of common test methods. DNA hybridization is commonly analyzed in this manner, using an appropriate fluorophore coupled to a set of known oligonucleotides that hybridize to capture oligonucleotides affixed to a slide. Sandwich immunoassays also employ this method of analysis, either using a tagged secondary antibody that binds to a primary antibody, or using a secondary biotinylated antibody and an avidin-fluorophore as the tag. Many variations on this method are well known.

Various other types of light interference may occur in fluorescent detection. Light scatter occurs by reflection of the excitation beam, while light dispersion occurs by reflection and bending of the excitation beam. Scatter and dispersion may represent a large part of the light striking a detector. In general, when a substance (such as a protein, nucleic acid or other biomolecule) is affixed to the surface of a glass slide, it acts as a mirror to reflect and scatter light in a variety of directions. The amount of surface covered and the mass or density of the attached material may greatly affect the amount of scattered light. The chemical composition of proteins, oligonucleotides or polymers attached to the glass surface may also affect the scattered light, as seen in FIG. 8 below. In addition, the material attached to the glass surface material may itself fluoresce. The glass used may also have surface irregularities that can affect the signals received by the detector. The energy absorbed across the glass may vary from one spot to another, making signal analysis very problematic. Such problems require the use of novel methods of fluorescent detection and/or data analysis.

Evanescent Emission and Scattered Light

Evanescent signals are generally very weak and light scatter is intense, making accurate quantitative detection of analytes problematic. Light scatter is frequently assumed to be eliminated by filters. However, scattered light is almost always present and can be a significant part of the total signal reaching a detector. Filters used to remove light scatter also remove much of the target emission signal, thereby decreasing detector sensitivity. Filters may also transmit a small amount of scattered light. If the scattered light is relatively large compared to the evanescent emitted light, the detected signal will be a combination from several sources, only one of which represents target molecule binding.

The components of light scattering are illustrated in FIG. 8. Two spots (e.g., different antibodies) are deposited on a glass surface. During a method to detect a target, one of the spots remains totally non-reactive. The other spot reacts with a target, such as a bacterial pathogen and/or other reagents. Target binding to the reactive antibody increases the mass attached to the spot and results in a larger surface area and a change in molecular structure at the spot. A mass effect has occurred. The light scatter from the reactive spot will be different from the light scatter before target molecule binding. A sensitive photon-counting detector could detect this difference in scatter. A variety of instruments, such as certain flow cytometers and turbidity meters take advantage of scatter to quantify the amount of material in a solution. Those instruments measure the angle of scatter for a beam of light impinging on a target material. The change in signal is the difference between the reference signal (S_{ref}) and signal 2 (S₂). In FIG. 8, the S₂ signal is shown as having two components, a modified scatter signal plus a mass effect signal of the coupled pathogen. The signal from the reactive spot changes while the signal from the non-reactive spot signal is constant.
ΔS(non-reactive spot)=0
ΔS(reactive spot)=Modified(Sₚ)+M₁−S_{ref}

If the mass effect is sufficient to cause a large scatter effect, the fluorophore used for target detection could be eliminated. For example in DNA hybridization experiments, the mass attached to a surface using standard oligonucleotide probes (about 24 nucleotides in length) may be increased by a factor of 2 or more upon binding of target nucleic acids. Such a large change in mass may be detectable by monitoring light scatter instead of evanescent waves. In the case of a sandwich immunoassay with a biotinylated secondary antibody, another mass effect occurs when the biotinylated antibody binds to the pathogen. A third mass effect occurs when avidin-conjugated fluorophore binds to biotin.

The most sensitive signal may be obtained by subtracting the initial reference signal from the final captured signal, obtained after the fluorophore has been attached and excited. That signal represents the modified accumulated mass effects and the emission signal for the reactive spot.
ΔS(reactive spot)=Modified accumulated mass effects+Emission−S_{ref}

This method of analysis can be used with a CMOS imager or any known digital imaging method that allows storage of pixel images for subsequent processing. The signal obtained from each spot will contain more useful information and will show a more intense change upon target binding if a proper subtraction method is used. The scatter effect may be turned to an advantage in detecting target binding. Moreover, it is unnecessary to have fluorophore emission and absorption curves well separated, since spurious signals are subtracted out of the image. The full intensity of an emission signal may be measured without reducing emitted light by with filters.

A subtraction method also eliminates artifacts and defects that may derive, for example, from inhomogeneity (chips, flaws) in the glass slide surface. The non-reactive spots completely blank out and do not appear as a signal. Because CMOS imagers and pixel capturing devices in general exhibit a random, very low level noise there are limits as to what kinds of signals can be detected. At any given point in time, the baseline reference may exhibit a random number of spikes. A weak signal falling between two spikes would not normally be detected against this background noise.

The signal-to-noise problem may be improved if numerous images are captured and added one upon the other. Because the random spikes inherent in a detector such as a CMOS imager are constantly shifting about, accumulating the frame images will tend to average out the random noise. However a weak signal from the emission of an excited fluorophore does not change its pixel location. Therefore, an accumulated signal caused by target binding will increase with time. This method is similar to taking a photoimage of a distant star or galaxy, by tracking the object as it moves across the sky. The object of interest appears brighter against the background with time because the signal has accumulated at the same spot on the detector, while the background light averages out.

Method of Analysis

In an exemplary embodiment, a glass or nylon slide or other matrix array is secured on a stage. Before target molecule binding, an excitatory laser is focused on one end of the slide at an inclined angle about 30 to 40 degrees. The slide acts as a waveguide to conduct the excitatory light to spots, containing bound primary antibody, on the surface. A CMOS, CCD or other optical imager is used to capture the light signals. The imager may be located beneath the slide and aligned so that spots on the slide are directly above the imager and are sharply focused on the imager surface with optical lenses and apertures.

A number of pictures are taken. Each picture represents a single frame. For example 10 frames are taken using a 50 millisecond exposure. The exposure is selected so that the amount of light captured in a single frame is within the sensitive range for the camera. The 10 digital frames are then added to provide a reference set that is used for subtraction of unwanted (background) signals. The accumulated image is referred to as the calibration slide.

The same number of frames used to obtain the reference slide image are taken of the sample slide after binding of primary and/or secondary antibodies, enzyme and reagent, etc, using the same exposures. The cumulative set of frames is referred to as the sample slide image. The luminescent signal for each spot is determined by subtracting the reference slide image from the sample slide image. This process essentially eliminates background noise and matrix array artifacts, resulting in very sensitive detection of target molecules.

In alternative embodiments, pictures may be obtained in either still frame or video mode. A typical video frame runs at 2000 ms and captures 100 frames each for the reference and sample analysis. This method removes artifacts and non-reactive spots, leaving only those signals that represent target molecule binding to the array.

Auto-Threshold Correction

A common problem in sample analysis has to do with making measurements and interpreting the meaning of values when no absolute standard exists. For example in measuring luminosity, because the measurement can not normally be obtained precisely when the reaction is initiated, and because the concentration of substrate is typically not known, only relative luminosity measurements are generally obtained and used in the analytic process. There are no absolute standards against which the signal can be measured.

Luminosity is frequently measured using a CCD camera with software. Each instrument will measure in terms of the absolute numeric value differently because of variations in the circuitry, optics, CCD chip and housing, etc. Even the same instrument will measure differently from moment to moment and scan to scan and on different days due to subtle changes in temperature and circuitry response, variations in solutions, scanning time, etc. making it difficult to interpret absolute values obtained in a given set of measurements.

For a particular instrument, when these differences in signal are relatively small compared to the numeric value obtained it may be possible to easily differentiate one sample in a set relative to others in the set, and to assign it to a population with a different distribution profile, for example as a negative or positive serum sample for a particular marker. However, in practice the differences in a measurement may be large from one experiment to the next relative to the absolute numeric value for the same or similar samples. This is especially true when the relative luminosity is very low in value and low level measurements are made.

By using a reference within a set of measurements, to which all other measurements are compared, it is possible to accurately state that a signal is either greater than, equal to, or less than that reference numeric value. And with the frequently observed broad dynamic range of over 1000 to a million in relative luminosity units, it is possible to measure relative luminosities to well within a few parts per 1000 units.

But with each new set of measurements, due to the change in absolute values, the relative luminosities of one set of measurements obtained by subtracting background measurement, or specific sample measurement, from that of another will not necessarily render the equivalent relative luminosity for a test sample. A method of differentiating one sample set in a series (the negative or normal set of sera) with a distribution curve that is different from that of another (positive or uniquely marked and differentiated sample set) is needed. And a method of making all instruments equivalent in their ability to differentiate signals in the same way is also needed. This can be done for any sample test, using only the measured luminosity for sample providing either the negative or positive reference set and background reference signal is also obtained in the scanning procedure.

Generally it is better to use as the reference the negative or normal set because a pool of samples may be prepared and used as the reference, closely approximating the average expected value for several samples simultaneously tested and averaged. Positive or uniquely marked sera may vary considerably depending upon the marker. For example, cattle infected with M. bovis with circulating antibody as the marker of interest may have very little antibody early in the infection. In a different animal or at a later time in the infection the antibody concentration may be very much larger or smaller in concentration. The positively infected animals should not be used for referencing. Negative (normal) sera are devoid of the antibody marker specific for M. bovis and can be used as the reference.

Relative Luminosity Measurements

The Total Optical Assay Device (TOAD) described above, used with software, optics, and instrument housing loaded with a set of samples for analysis provides to the investigator a numeric value for each sample in a crib set. A crib set normally contains 12 or more wells, each containing either sample or a control solution that is scanned to capture an image in a set period of time (e.g., 10 minutes).

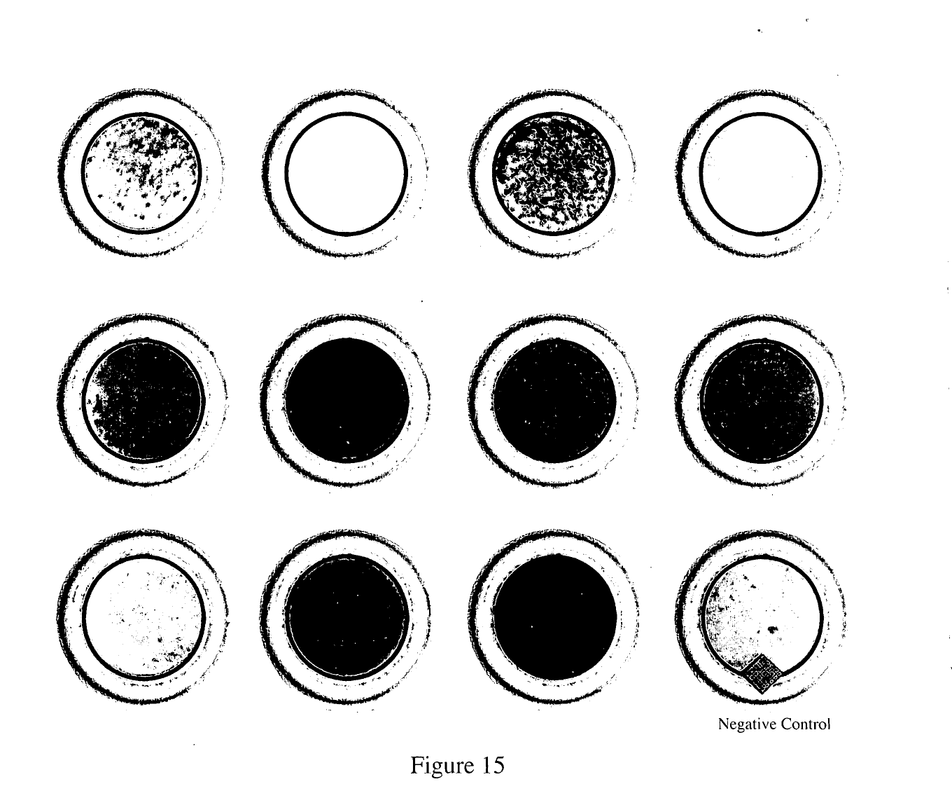

Normally several samples are scanned simultaneously. Background numeric values representing signal that would have been observed in the absence of sample (background) are obtained at the same time as samples are scanned by examining those areas in the CCD camera target area between the samples. The difference between the sample numeric value and background numeric value represents the relative luminosity for a particular test sample. A representative image for a set of 12 samples is shown in FIG. 15 with the pool of approximately 10 sera negative animals at the same substrate concentration used as a reference (negative control).

Because only 11 samples and the negative control can be processed in a single crib set assay, many sets are required to analyze a large number of samples. For each set, 11 additional samples are obtained and their numeric values are measurable relative to the negative control reference for that crib set. Table 5 shows the results for 9 consecutive assay sets with the relative luminosity for 99 different samples in a blinded study with uninfected and infected cattle sera.

The relative luminosity for each sample in a crib set provides useful information allowing the differentiation of a positive from a negatively infected animal using the referenced pool of negative controls. This is accomplished by assigning a threshold above which the sample is positive and below which it is negative. The threshold is obtained empirically for a given set of experimental conditions.

Threshold Assignments:

The threshold cutoff used to determine if a sample is either positively or negatively infected is determined empirically. Negative control reference values were obtained for each of the 9 crib sets and the background, NC, and NC relative luminosity values (by subtracting BG from NC) are shown in Table 6.

The relative luminosity for pooled negative control sera is acquired (see NC_RL Table 6). The values for NC_RL vary considerably. The ratio of the average of several sera measured individually plus a multiple standard deviation from the average relative to the pooled negative reference is a constant. The ratio is constant because the CCD image out put is directly proportional to photons striking its surface, and both the pooled sera reference and negative sample sera in a test are therefore affected to the same degree for a set of experimental conditions.

A threshold is determined automatically by exploiting the relationship between the average expected relative luminosity value for a serum that is negative relative to the reference of pooled negative samples, and the observed value for a sample that may or may not be truly negative by adding to the average expected relative luminosity value a multiplier of standard deviations above the average that should include any negative sera sample. This can be expressed by the equation:
Threshold=Obs_{NC}+X(SD)−NC_RL pooled reference
   Obs_{NC}=Observed negative control sample serum value
   X=Multiplier
   SD=Standard Deviation of several negative sample sera values
   NC_RL=Negative Control pooled sera reference value

If the observed value for a sample is equal to the NC pooled reference, then the threshold is just the multiplier of standard deviations above the pooled reference for a given set of experimental conditions.
Threshold=X(SD)

Since the relationship between the average of several tested negative samples plus a multiplier of standard deviations for those samples tested relative to the pooled reference test sample is a constant, the threshold value can be automatically calculated by a simple formula as shown below.
{Obs_{Nc-average}+X(SD)}/NC_{—}RL_{pooled reference}=Constant
Therefore,
Threshold={Constant−1}NC_{—}RL
Threshold Factor={Constant−1}

A particular sample in a crib set is determined to be either positive or negative because its value is either above or below the threshold.
Positive Assignment Obs_{relative luminosity}≧Threshold
Negative Assignment Obs_{relative luminosity}<Threshold

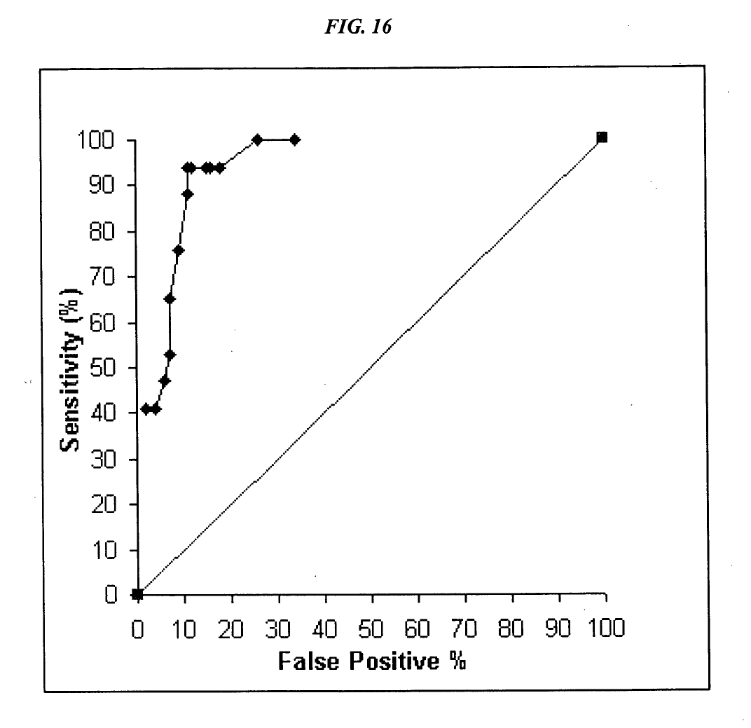

In practice the value for {Constant−1} is allowed to vary after testing so that the effect on Specificity and Sensitivity measurements can be determined. The standard ROC curve is then constructed (see, e.g., FIG. 16). From the table of values, the threshold factor is selected for any Specificity and Sensitivity cut off desired (Table 7). For example a threshold factor at 0.9 represents Specificity and Sensitivity values of 91 and 76% respectively.

Normalizing Thresholds and Relative Luminosities

The calculated threshold is used to assign a sample based on its relative luminosity to either the positively infected or normal sera groups. If a threshold factor used results in 100% sensitivity, all of the positive samples should be identified (i.e., false negatives should be zero) but there may be a number of false positives (normal sera group) because the threshold is too low to eliminate their inclusion. The false positives can be eliminated in subsequent experiments with reflex supplemental testing.

By normalizing the relative luminosities against the selected threshold, a sample may be identified as either positive or negative because its value is either greater or less than 1.0 respectively. This is a more useful way of expressing values because it makes it easier to examine a list of samples to immediately see to which set the assignment should be allotted based on the numeric value for that sample.

The threshold is converted (normalized) from a relative luminosity value to an integer by dividing the observed relative luminosity for a sample by the threshold for that crib set. It can be appreciated at a glance when comparing one sample to the other that the ratio Delta/threshold is above or below 1.0. This alone determines the assignment for a positive or negative sample. Relative luminosity inspection by itself does not allow for such an easy interpretation of the assignment.
Positive Assignment {Obs_{relative luminosity}}/Threshold≧1.0
Negative Assignment {Obs_{relative luminosity}}/Threshold<1.0

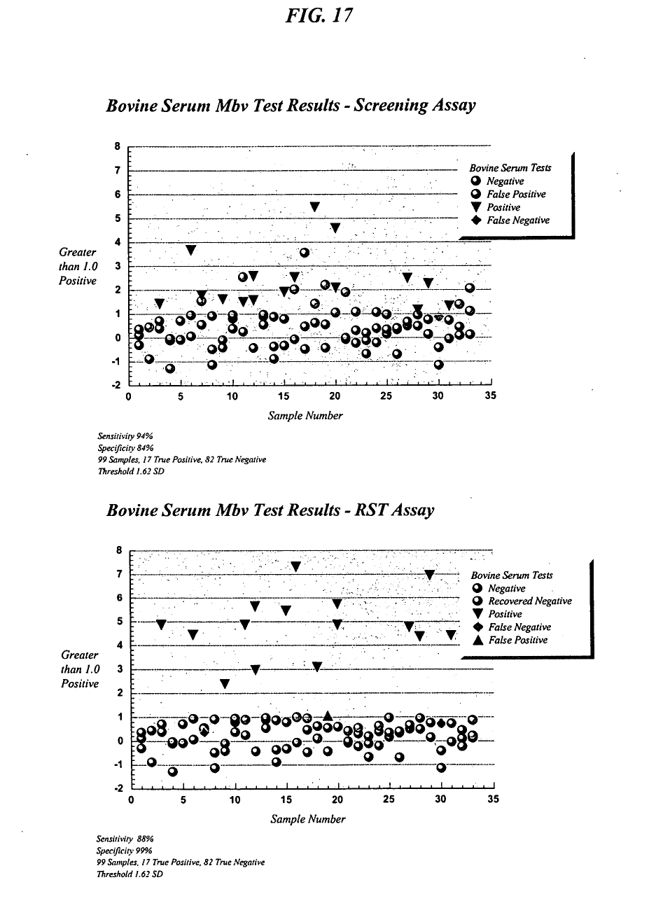

The automated and normalized values for the 99 tested animals are listed in Table 8, for the initial round of testing only. Graphic representations for screening and reflex supplemental testing are shown in FIG. 17.

By auto calculating the threshold, the criteria for sensitivity and specificity are set for a given set of experimental conditions. Each instrument will by the same criteria interpret results in an equivalent manner. And by normalizing the observed relative luminosity by dividing by the threshold calculated relative luminosity, all samples in a series or from instrument to instrument are easily assessed as either positive or negative for the threshold selected as they are either greater than or less than 1.0.

General Methods for Proteins and Peptides

A variety of polypeptides or proteins may be used within the scope of the claimed methods and compositions for detection of pathogens and/or marker molecules. In certain embodiments, the proteins may comprise antibodies or fragments of antibodies containing an antigen binding site. Many antibodies with binding specificities for individual analytes are known in the art.

As used herein, a protein, polypeptide or peptide generally refers, but is not limited to, a protein of greater than about 200 amino acids, up to a full length sequence translated from a gene; a polypeptide of greater than about 100 amino acids; and/or a peptide of from about 3 to about 100 amino acids. For convenience, the terms “protein,” “polypeptide” and “peptide” are used interchangeably herein. Accordingly, the term “protein or peptide” encompasses amino acid sequences comprising at least one of the 20 common amino acids found in naturally occurring proteins, or at least one modified or unusual amino acid, including but not limited to those shown on Table 9.

Proteins or peptides may be made by any technique known to those of skill in the art, including the expression of proteins, polypeptides or peptides through standard molecular biological techniques, the isolation of proteins or peptides from natural sources, or the chemical synthesis of proteins or peptides. The nucleotide and protein, polypeptide and peptide sequences corresponding to various genes have been previously disclosed, and may be found at computerized databases known to those of ordinary skill in the art. One such database is the National Center for Biotechnology Information's Genbank and GenPept databases (www.ncbi.nlm.nih.gov/). The coding regions for known genes may be amplified and/or expressed using the techniques disclosed herein or as would be know to those of ordinary skill in the art. Alternatively, various commercial preparations of proteins, polypeptides and peptides are known to those of skill in the art.

Fusion Proteins

Other embodiments may concern fusion proteins, such as the CP10_ESAT fusion protein discussed below. These molecules generally have all or a substantial portion of a peptide, linked at the N- or C-terminus, to all or a portion of a second polypeptide or protein. For example, fusions may employ leader sequences from other species to permit the recombinant expression of a protein in a heterologous host. Another useful fusion includes the addition of an immunologically active domain, such as an antibody epitope. Certain types of fusion proteins may employ antigenic epitopes from two or more different proteins, in order to provide for a broader spectrum of antibody screening. Use of fused epitopes from multiple pathogen proteins may be expected to provide increased sensitivity for detection of anti-pathogen antibodies in an infected host. Methods of generating fusion proteins are well known to those of skill in the art. Such proteins can be produced, for example, by chemical attachment using bifunctional cross-linking reagents, by de novo synthesis of the complete fusion protein, or by attachment of a DNA sequence encoding a first protein or peptide to a DNA sequence encoding a second peptide or protein, followed by expression of the intact fusion protein.

Protein Purification

In certain embodiments a protein or peptide may be isolated or purified. Protein purification techniques are well known to those of skill in the art. These techniques involve, at one level, the homogenization and crude fractionation of the cells, tissue or organ to polypeptide and non-polypeptide fractions. The protein or polypeptide of interest may be further purified using chromatographic and electrophoretic techniques to achieve partial or complete purification (or purification to homogeneity). Analytical methods particularly suited to the preparation of a pure peptide are ion-exchange chromatography, gel exclusion chromatography, polyacrylamide gel electrophoresis, affinity chromatography, immunoaffinity chromatography and isoelectric focusing. A particularly efficient method of purifying peptides is fast protein liquid chromatography (FPLC) or even HPLC.

Various techniques suitable for use in protein purification are well known to those of skill in the art. These include, for example, precipitation with ammonium sulphate, PEG, antibodies and the like, or by heat denaturation, followed by: centrifugation; chromatography steps such as ion exchange, gel filtration, reverse phase, hydroxylapatite and affinity chromatography; isoelectric focusing; gel electrophoresis; and combinations of these and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater“-fold” purification than the same technique utilizing a low pressure chromatography system. Methods exhibiting a lower degree of relative purification may have advantages in total recovery of protein product, or in maintaining the activity of an expressed protein.

Affinity chromatography is a chromatographic procedure that relies on the specific affinity between a substance to be isolated and a molecule to which it can specifically bind. This is a receptor-ligand type of interaction. The column material is synthesized by covalently coupling one of the binding partners to an insoluble matrix. The column material is then able to specifically adsorb the substance from the solution. Elution occurs by changing the conditions to those in which binding will not occur (e.g., altered pH, ionic strength, temperature, etc.). The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand.

Synthetic Peptides

Proteins or peptides may be synthesized, in whole or in part, in solution or on a solid support in accordance with conventional techniques. Various automatic synthesizers are commercially available and can be used in accordance with known protocols. See, for example, Stewart and Young, (1984); Tam et al., (1983); Merrifield, (1986); and Barany and Merrifield (1979). Short peptide sequences, usually from about 6 up to about 35 to 50 amino acids, can be readily synthesized by such methods. Alternatively, recombinant DNA technology may be employed wherein a nucleotide sequence which encodes a peptide of interest is inserted into an expression vector, transformed or transfected into an appropriate host cell, and cultivated under conditions suitable for expression.

Antibodies

The term “antibody” is used to refer to any antibody-like molecule that has an antigen binding region, and includes antibody fragments such as Fab′, Fab, F(ab′)₂, single domain antibodies (DABs), Fv, scFv (single chain Fv), and the like. Techniques for preparing and using various antibody-based constructs and fragments are well known in the art. Means for preparing and characterizing antibodies are also well known in the art (See, e.g., Antibodies: A Laboratory Manual, Cold Spring Harbor Laboratory, 1988). Antibodies of use for detection of various pathogens of interest may also be commercially obtained from a wide variety of known sources. For example, a variety of antibody secreting hybridoma lines are available from the American Type Culture Collection (Rockville, Md.).

Polyclonal antibodies may be prepared by immunizing an animal with an immunogen and collecting antisera from that immunized animal. A wide range of animal species can be used for the production of antisera. Typically an animal used for production of anti-antisera is a non-human animal, for example, rabbits, mice, rats, hamsters, pigs or horses. Because of the relatively large blood volume of rabbits, a rabbit is a preferred choice for production of polyclonal antibodies.

Antibodies, both polyclonal and monoclonal, may be prepared using conventional immunization techniques, as will be generally known to those of skill in the art. A composition containing antigenic epitopes can be used to immunize one or more experimental animals, such as a rabbit or mouse, which will then proceed to produce specific antibodies. Polyclonal antisera may be obtained, after allowing time for antibody generation, simply by bleeding the animal and preparing serum samples from the whole blood.

As is well known in the art, a given composition may vary in its immunogenicity. It is often necessary, therefore, to boost the host immune system, as may be achieved by coupling an immunogen to a carrier. Exemplary and preferred carriers are keyhole limpet hemocyanin (KLH) and bovine serum albumin (BSA). Other albumins such as ovalbumin, mouse serum albumin or rabbit serum albumin also can be used as carriers. Means for conjugating an antigen to a carrier protein are well known in the art and include glutaraldehyde, m-maleimidobenzoyl-N-hydroxysuccinimide ester, carbodiimide and bis-biazotized benzidine.

As also is well known in the art, the immunogenicity of a particular immunogen composition can be enhanced by the use of non-specific stimulators of the immune response, known as adjuvants. Exemplary and preferred adjuvants include complete Freund's adjuvant (a non-specific stimulator of the immune response containing killed Mycobacterium tuberculosis), incomplete Freund's adjuvants and aluminum hydroxide adjuvant.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster, injection also may be given. The process of boosting and titering is repeated until a suitable titer is achieved. When a desired level of immunogenicity is obtained, the immunized animal can be bled and the serum isolated and stored, and/or the animal can be used to generate monoclonal antibodies.

Monoclonal antibodies may be readily prepared through use of well-known techniques, such as those exemplified in U.S. Pat. No. 4,196,265. Typically, this technique involves immunizing a suitable animal with a selected immunogen composition. The immunizing composition is administered in a manner effective to stimulate antibody producing cells. Cells from rodents such as mice and rats are preferred, however, the use of rabbit, sheep or frog cells is also possible. Mice are preferred, with the BALB/c mouse being most preferred as this is most routinely used and generally gives a higher percentage of stable fusions.

Following immunization, somatic cells with the potential for producing antibodies, specifically B-lymphocytes (B-cells), are selected for use in the mAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible. Often, a panel of animals will have been immunized and the spleen of the animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

Any one of a number of myeloma cells may be used, as are known to those of skill in the art. For example, where the immunized animal is a mouse, one may use P3-X63/Ag8, P3-X63-Ag8.653, NS1/1.Ag 4 1, Sp210-Ag14, OF, NSO/U, MPC-11, MPC11-X45-GTG 1.7 and S194/5XX0 Bul; for rats, one may use R210.RCY3, Y3-Ag 1.2.3, IR983F and 4B210; and U-266, GM1500-GRG2, LICR-LON-HMy2 and UC729-6 are all useful in connection with cell fusions.

Methods for generating hybrids of antibody-producing spleen or lymph node cells and myeloma cells usually comprise mixing somatic cells with myeloma cells in a 2:1 ratio, though the ratio may vary from about 20:1 to about 1:1, respectively, in the presence of an agent or agents (chemical or electrical) that promote the fusion of cell membranes. Fusion methods using Sendai virus, and those using polyethylene glycol (PEG), such as 37% (v/v) PEG, have been described. The use of electrically induced fusion methods is also appropriate.

Fusion procedures usually produce viable hybrids at low frequencies, around 1×10⁻⁶ to 1×10⁻⁸. However, this does not pose a problem, as the viable, fused hybrids are differentiated from the parental, unfused cells (particularly the unfused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine.

A preferred selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B-cells can operate this pathway, but they have a limited life span in culture and generally die within about two wk. Therefore, the only cells that can survive in the selective media are those hybrids formed from myeloma and B-cells.

This culturing provides a population of hybridomas from which specific hybridomas are selected. Typically, selection of hybridomas is performed by culturing the cells by single-clone dilution in microtiter plates, followed by testing the individual clonal supernatants (after about two to three wk) for the desired reactivity. The assay should be sensitive, simple and rapid, such as radioimmunoassays, enzyme immunoassays, cytotoxicity assays, plaque assays, dot immunobinding assays, and the like.

The selected hybridomas would then be serially diluted and cloned into individual antibody-producing cell lines, which clones can then be propagated indefinitely to provide mAbs. The cell lines may be exploited for mAb production in two basic ways. A sample of the hybridoma can be injected (often into the peritoneal cavity) into a histocompatible animal of the type that was used to provide the somatic and myeloma cells for the original fusion. The injected animal develops tumors secreting the specific monoclonal antibody produced by the fused cell hybrid. The body fluids of the animal, such as serum or ascites fluid, can then be tapped to provide mAbs in high concentration. The individual cell lines also could be cultured in vitro, where the mAbs are naturally secreted into the culture medium from which they can be readily obtained in high concentrations. mAbs produced by either means may be further purified, if desired, using filtration, centrifugation, and various chromatographic methods such as HPLC or affinity chromatography.

### EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Exemplary Protocols for Bovine Tuberculosis Testing

As discussed above, a capture probe comprising one or more peptide sequences or other antigens expressed in a target pathogen of interest may be used to bind to and detect the presence of anti-pathogen antibodies in blood, serum or other sample obtained from a subject. The skilled artisan will realize that the target to be detected is not limited, and any target against which circulating host antibodies are present may be detected using similar protocols. Conversely, antibodies against a target of interest may be used as capture probes and the presence of target antigen detected, for example, by sandwich ELISA or other techniques well known in the art.

Materials and Methods

Production of Recombinant Antigen (RecAg) Probe

In the present exemplary embodiment of bovine tuberculosis detection, the capture probe used was a recombinant fusion protein expressed from two adjacent open reading frames (ORFs) within the Mycobacterium tuberculosis strain H37Rv genome, encoding CFP-10 and ESAT-6. Those two gene products were fused in frame by a process of PCR-directed mutagenesis in which the TGA stop codon of CFP-10 was changed to Gly (GGA) and an additional G residue was inserted downstream within the 32 nucleotide intervening region between CFP-10 and ESAT-6 in order to retain correct reading frame. This resulted in a fuision protein consisting of the complete CFP-10 gene product (100 amino acids) fused to the complete ESAT-6 gene product (95 amino acids) with an intervening 12 “nonsense” amino acids from the inter-ORF region. The recombinant antigen (RecAg) was then affixed to ferrite beads as a probe for bovine antibodies in infected animals.

| | |
|---|---|
| CFP-10 Amino Acid Sequence | |
| MAEMKTDAATLAQEAGNFERISGDLKTQIDQVESTAGSLQGQWRGAAGTAAQA | (SEQ ID NO: 1) |
| AVVRFQEAANKQKQELDEISTNIRQAGVQYSRADEEQQQALSSQMGF | |
| CFP-10 Nucleotide Sequence | |
| atggcagagatgaagaccgatgccgctaccctcgcgcaggaggcaggtaatttcgagcggatctccggcgacctgaaaaccc | (SEQ ID NO: 2) |
| agatcgaccaggtggagtcgacggcaggttcgttgcagggccagtggcgcggcgcggcggggacggccgcccaggccgc | |
| ggtggtgcgcttccaagaagcagccaataagcagaagcaggaactcgacgagatctcgacgaatattcgtcaggccggcgtc | |
| caatactcgagggccgacgaggagcagcagcaggcgctgtcctcgcaaatgggcttcTGA | |
| Inter-ORF region | |
| cccgctaatacgaaaagaaacggagcaaaaac | (SEQ ID NO: 3) |
| ESAT-6 Amino Acid Sequence | |
| MTEQQWNFAGIEAAASAIQGNVTSIHSLLDEGKQSLTKLAAAWGGSGSEAYQG | (SEQ ID NO: 4) |
| VQQKWDATATELNNALQNLARTISEAGQAMASTEGNVTGMFA | |
| ESAT-6 Nucleotide Sequence | |
| atgacagagcagcagtggaatttcgcgggtatcgaggccgcggcaagcgcaatccagggaaatgtcacgtccattcattccctc | (SEQ ID NO: 5) |
| cttgacgaggggaagcagtccctgaccaagctcgcagcggcctggggcggtagcggttcggaggcgtaccagggtgtccag | |
| caaaaatgggacgccacggctaccgagctgaacaacgcgctgcagaacctggcgcggacgatcagcgaagccggtcaggc | |
| aatggcttcgaccgaaggcaacgtcactgggatgttcgcatag | |
| Entire Nucleotide Sequence | |
| atggcagagatgaagaccgatgccgctaccctcgcgcaggaggcaggtaatttcgagcggatctccggcgacctgaaaaccc | (SEQ ID NO: 6) |
| agatcgaccaggtggagtcgacggcaggttcgttgcagggccagtggcgcggcgcggcggggacggccgcccaggccgc | |
| ggtggtgcgcttccaagaagcagccaataagcagaagcaggaactcgacgagatctcgacgaatattcgtcaggccggcgtc | |
| caatactcgagggccgacgaggagcagcagcaggcgctgtcctcgcaaatgggcttcGGAcccgctGaatacgaaaaga | |
| aacggagcaaaaacatgacagagcagcagtggaatttcgcgggtatcgaggccgcggcaagcgcaatccagggaaatgtca | |
| cgtccattcattccctccttgacgaggggaagcagtccctgaccaagctcgcagcggcctggggcggtagcggttcggaggcg | |
| taccagggtgtccagcaaaaatgggacgccacggctaccgagctgaacaacgcgctgcagaacctggcgcggacgatcagc | |
| gaagccggtcaggcaatggcttcgaccgaaggcaacgtcactgggatgttcgcatag | |

As indicated, the TGA stop codon at the 3′ end of the CFP-10 coding sequence was mutagenized to a glycine encoding GGA glycine codon. Also a G residue was inserted 6 bases downstream from the mutagenized GGA codon to make the fused ESAT-6 coding sequence in-frame with the CFP-10 sequence. Recombinant CFP-ESAT antigen was prepared by GenWay Biotech, Inc. (San Diego, Calif.).

Conjugation of Magnetic Beads to Fusion Protein

The CFP-10:ESAT-6 recombinant fusion protein antigen (CFP-ESAT) was conjugated to ferrite beads using a PolyLink Protein Coupling Kit for COOH Microspheres from Bangs Laboratories, Inc. (Fishers, Ind.) with slight modifications in the coupling process. Fusion protein free of Protein A and other interfering agents was covalently coupled to acrylic carboxyl (COOH) coated ferrite beads. Briefly, the carboxyl groups were activated with water-soluble carbodiimide with a NHS modification, which then reacts with these groups to create an active ester. The ester is reactive toward the primary amines on the protein to be conjugated to the beads. A representative protocol is disclosed in Data Sheet #644 (which accompanies the PolyLink Protein Coupling Kit).

The stock ferrite bead-CFP-ESAT conjugate, after blocking and washing in a glycinamide buffer, was suspended to a 2 mg/ml ferrite bead concentration in a standard dilution buffer with a microbial inhibitor. Prior to use, the beads were diluted to a concentration of 0.04 mg/ml in Standard Dilution Buffer (10 mM PBS, 0.1% BSA, 0.05% ProClin) for use in the bovine TB assay. Aliquots of the diluted bead preparation were used to spike diluted sera samples for each sample analyzed.

Materials

Carboxyl (COOH) coated ferrite microspheres and the PolyLink Protein Coupling Kit were from Bangs Laboratories, Inc., (Fishers Ind.). Biotinylated goat anti-bovine IgM (mu chain specific) was from KPL (Gaithersburg, Md.). HRP-SA solution, SuperSignal West Pico Luminol/Enhancer Solution and SuperSignal West Pico Stable Peroxide Solution were from Pierce (Rockford, Ill.).

Protocol

An exemplary step by step protocol for a rapid diagnostic test for Mbv Infection is provided below. More generally, in a typical test, a 1:50 dilution of a serum sample was made by adding 20 μl whole bovine serum (preferably azide-free) to 980 μl of Standard Dilution Buffer (10 mM PBS, 0.1% BSA, 0.05% ProClin) in a 1.5 ml Eppendorf Protein LoBind Tube. Then 280 μl of this sample was dispensed into a second Eppendorf LoBind tube and 70 μl of a well dispersed solution of 0.05 mg/ml ESAT-6/CFP-10 Bangs Biomag bead conjugate was pipetted into the bovine serum sample and incubated at RT for 15 minutes in a non-magnetic rack position.

The tube was then placed snugly into a magnetic rack and 720 μl of wash buffer (10 mM PBS, 0.05% Tween 20) was added to the sample, which was then incubated in the magnetic force field for an additional 2 minutes. With the sample tube remaining in the magnetic rack, all sample liquid was carefully removed by dropping an L-1000 pipette tip to bottom of tube and aspirating all of the buffer and sample (leaving behind two discrete dots of ferrite beads “snake-eyes” firmly affixed to the container walls in the force field). The tube was then removed from the rack and the beads were washed with 900 μl of a PBS-0.05% Tween wash buffer. Again the sample was placed in the force field for 2 minutes to affix beads. The wash step was again repeated so that 3 washes (the initial 720 μl spike and the 2 subsequent washes) were achieved. Then 250 μl of dialyzed KPL (Mu chain specific) biotinylated anti-bovine IgM (0.3 μg/ml in Standard Dilution Buffer) was used to wash beads from the sample wall back into solution. The beads were incubated at RT for 10 minutes with biotinylated antibody.

The sample was again spiked with 720 μl PBS-Tween wash buffer, incubated on magnets and washed as described before for the first sample incubation step. The tube containing only ferrite beads was then again removed from the magnetic force field and 200 μl of HRPSA (0.2 μg/ml in Standard Dilution Buffer) was used to disperse beads into solution where they incubate for 5 minutes at RT. Finally, using the identical process the sample was spiked with 720 μl PBS-Tween wash buffer and washed twice by dispersing and affixing beads at 2 minute intervals. Then 200 μl of a 1:1 mixture of luminal peroxide (Pierce) was added to each tube, uniformly dispersing beads into the solution.

The entire 200 μl luminol-peroxide-bead complex was transferred into a clear break-away well and deposited into a 12 sample crib (one sample was always a negative control sample consisting of a pooled collection of bovine sera which were tested negative for TB) for imaging. Emitted photons are captured for 10 minutes on the TOAD™ apparatus described above. Observed values for each sample, including negative control as well as background (determined as the average signal on 4 background fields) were then software processed with auto-threshold assignments. Results were determined by the program as Mbv negative or positive. Positives were retested in two Reflex rounds as described above. This general procedure may be followed or modified by routine experimentation according to techniques well known in the art. A more detailed exemplary step-by-step protocol of use in the claimed methods is described below.

Detailed Protocol

The skilled artisan will realize that the detailed protocol steps listed below represent a preferred embodiment of the claimed methods and that various steps, concentrations, amounts, times, etc. may be varied by routine experimentation within the scope of the claimed methods. All serum dilutions should preferably be made and all testing conducted in 1.5 ml Eppendorf Protein LoBind tubes. One negative control sample was included in each (12 sample) crib set. A 280 μl aliquot of a pooled collection of bovine sera (1:50 dilution) which have tested negative for TB was used in place of an individual serum sample and processed exactly the same as the bovine samples being tested.

Inclusion of a positive control is optional, but it is a good idea to run periodically for quality control testing of assay reagents. Fifty μl of a solution of 0.04 mg/ml BBSA conjugated Bangs Biomag beads made up in Standard PriTest Dilution Buffer (10 mM PBS, 0.1% BSA, 0.05% ProClin) was dispensed into a 1.5 ml Eppendorf Protein LoBind tube. An additional 280 μl of Dilution Buffer was added to bring up volume for adherence of beads to magnets. The positive control was processed exactly like the other samples starting at Step 6 below (wash steps).
1) Make a 1:50 dilution of each serum sample to be tested by adding 20 μl whole bovine serum (without Na azide) to 980 μl of Dilution Buffer (10 mM PBS, 0.1% BSA, 0.05% ProClin) and aliquot 280 μl into an a 1.5 ml Eppendorf Protein LoBind tube.
2) Pipette 70 μl of a well dispersed solution of 0.05 mg/ml ESAT-6/CFP-10 Bangs Biomag bead conjugate into the 280 μl bovine serum sample and incubate at RT for 15 minutes. Place each tube snugly in a magnetic well and spike sample with 720 μl Wash Buffer (10 mM PBS, 0.05% Tween 20). Incubate on magnets for an additional 2 minutes to allow ‘snake eyes’, condensed patches of ferrite beads, to form on sides of tube exposed to magnets.
3) Carefully aspirate liquid from the tube without disturbing beads bound to the sides by placing an L-1000 pipette tip into the tube and suctioning fluid from bottom in a smooth, steady stream until air bubbles are observed in the tip. (One pipette tip was dedicated to one sample throughout the entire protocol).
4) After removing tube from magnetic wells, add 900 μl of Wash Buffer and gently mix beads back into solution by slowly pipetting solution in and out of the tip against the wall of tube (taking care to minimize foam production). Place tube back in magnetic well and incubate for 2 minutes. Pipette off fluid without disturbing bound beads as described above. Repeat this step 1 more time for a total of 3 washes (the initial spike and 2 subsequent washes).
5) After removing tube from magnetic well, add 250 μl of dialyzed KPL biotin-conjugated affinity purified goat anti-bovine IgM (mu chain specific) antibody prepared at 0.3 μg/ml in Dilution Buffer to each sample and disperse beads by gently hand vortexing into solution. Incubate at RT for 10 minutes, place snugly in magnetic wells, spike with 720 μl Wash Buffer and incubate for an additional 2 minutes.
6) Carefully pipette off liquid as in Step 3 with an additional 2 wash steps as described previously in Step 4. Remove sample tube(s) from magnetic wells.
7) Add 200 μl of Pierce HRP-SA (prepared at 0.2 μg/ml in Dilution Buffer) to each tube, disperse beads into solution and incubate RT for 5 minutes. Place snugly in magnetic wells, spike sample with 720 μl Wash Buffer and incubate for 2 minutes at RT
8) Carefully pipette off liquid and wash 2 times more with as described previously in step 4. Remove sample tube(s) from magnetic wells.
9) Prepare a solution of Pierce SuperSignal West Pico Luminol Enhancer and SuperSignal West Pico Stable Peroxide in a 1:1 ratio and add 200 μl to each sample tube. Vortex the contents of each tube gently by hand and squirt liquid down sides of tube with a L-200 pipette tip to disperse beads into solution. Transfer the entire 200 μl volume from each sample tube into a clear plastic breakaway well and deposit into a 12 sample crib (one sample of which is a negative control (1:50 dilution) of pooled Mbov negative sera).
10) Emitted photons are captured for 10 minutes on the TOAD™ apparatus described above and with analyzed with software under optimization conditions using a cooled CCD high resolution camera. Observed values for each sample and negative control as well as background (determined as the average of 4 background fields) are inserted into the Reflex-Testing—Automatic Threshold Adjustment Excel Sheet template. Results for each sample are analyzed by an algorithm in the excel template and given the designation of Mbov negative or positive. Positives in this first round of screening are retested exactly as described above (Reflex Test #1). Positives in this second round of testing are tested one final time (Reflex Test #2) for confirmation of positive status (M. bovis infection).
   Results

Results from three blinded Sensitivity and Specificity Studies obtained using the PriTest Rapid Diagnostic Test and Reflex Supplemental testing were pooled into one 99 sample study. Results from the initial screen are shown in Table 5. Assays were performed as described above, with Ferrite conjugate GW ESAT 70 ul, 0.04 mg/ml probe, 10 mM PBS 0.05% Tween 20 wash buffer, IgM 0.3 ug/ml 10 mM PBS 0.1% BSA 0.05% PC antibody solution, 0.2 ug/ml HRPSA, 10 mM PBS 0.1% BSA 0.05% PC, and 15, 10, 5 minute incubation cycles. The three studies exhibited an average sensitivity of 88, specificity 84, ROC 0.94, PPV 52 and NPV 96. A representative image for a single crib set of 11 sera and the negative control is shown in FIG. 15.

The advantages of reflex supplemental testing are shown in Table 8. It is clear that the reflex supplemental testing procedure decreases the number of false positive results obtained. More than 500 tests on sera have been completed. Approximately 20 positive samples and more negative sera samples have been used in a 1:50 dilution to identify factors that influence the effectiveness of the assay. Certain of these factors are discussed below.

Temperature Effects on Mbv Detection

The effect of temperature on the assay can significantly alter outcomes. Both lower temperature (˜4 C) and higher temperature (˜37 C) make it much more difficult to differentiate positive and negative serum samples from one another. At higher temperatures all samples including negative sera appear to react very strongly so that so much light was produced that the signals are essentially indistinguishable. Lowering the temperature has quite the opposite effect with signal loss for both negative and positive sera. The preferred temperature appears to be very near room temperature (˜25 C).

pH Effects on Mbv Detection

Based on limited analysis, optimal signals were detected at pH ˜7.4. Lowering the pH to 4.0 resulted in a marked decrease in signal for positive sera.

Biotinylated Secondary Antibody

Many secondary biotinylated antibodies have been tested in the assay including biotinylated IgY anti-bovine IgG, biotinylated goat IgG anti bovine IgG and biotinylated goat IgG anti-bovine IgM. Combinations have also been tested. All individual and combination tests have been successful in differentiating positive and negative sera to varying degrees, but the simplest and currently optimized antibody to use is biotinylated goat IgG anti-bovine IgM. The optimal concentration with 0.2 ug/ml HRP_SA was 0.3 ug/ml IgM in a standard dilution buffer.

Protein A Interference

Much effort was directed to prepare ferrite bead conjugates free of Protein A. Protein A was found to substantially interfere with assay development in generating false positive results and/or high background. Even trace amounts of Protein A present in solvent or substrate during RecAg conjugation resulted in beads that strongly react with biotinylated antibodies and bovine serum antibodies. With trace contamination the background signal for a negative control will be as bright or brighter than a positive serum test.

Protein A is commonly used for antibody purification schemes and is often co-eluted with the antibody and must be eliminated or false positives signals will be observed. This is true regardless of how the antibody will be used because Protein A binds to both Fc and Fab (to a lesser extent) allowing for bridging to other antibodies.

If the antibody is to be conjugated to ferrite beads and has Protein A in the mix, some of the Protein A will end up on the ferrite bead along with the antibody. Since we detect the antibody response on the ferrite as an antibody-antigen interaction typically with a biotinylated antibody against that same or linked target that the primary antibody has recognized, any biotin picked up (by the HRPSA reaction with luminol) would be assumed to be a confirmation that the primary antibody had picked up epitope. But with Protein A on the ferrite bead and a sensitive detector, the Protein A catches some of the biotinylated secondary antibody resulting in light detection at the end of the assay regardless of what happened to the primary antibody. A false positive is detected in the absence of epitope.

If the antibody is biotinylated (intended as a secondary antibody detector) and Protein A is present, some of the Protein A is biotinylated and because it binds with the Fc and Fab of the biotinylated antibody, ends up in the reagent even after dialysis. Because the Protein A can bridge between two antibodies linking them, even if no epitope is present the biotinylated antibody will be linked to the antibody on ferrites (or spotted on nitrocellulose) again resulting in a false positive detection.

Antibody Dialysis to Remove Protein A

Protein A's affinity for antibodies is dramatically reduced at lower pH. We use a 10 mM Glycine pH 2.8 dialysis and drive the dissociation by placing the antibody in a dialysis tube with 100 kD membrane cut-off and add to the Glycine a cationic resin to trap protein passing the membrane in the dialysate. Typically 1 gram of a strong cationic exchange resin is added to the Glycine pH 2.8, 1 liter exchange against 5 ml of antibody in the membrane tube (floatalyzer) and dialysis is performed 16 to 24 hours with 2 to 3 liter exchanges. The dialysate is switched to pH 7.4 10 mM PBS liter and continue dialysis an additional 16 to 24 hours with 2 to 3 liter exchanges. So the ratio is 200:1×3 in the Glycine buffer pH 2.8, and the same in the PBS media (million fold first and million fold second exchange ratios). If working with a biotinylated antibody, a 10 mM PBS 0.1% BSA 0.05% proclin (antimicrobial inhibitor) solution is used for the second set of exchanges. This results in very pure antibodies, in the first example in PBS ready for ferrite bead conjugation or biotinylation, and in the second example ready for dilution to an appropriate level for assay work.

Removal of Protein A from Proteins Between 10 kD and 100 kD in Size

Proteins in the category are usually epitopes. The immunoassay will not work properly if Protein A is present, as it will bind non-specifically to antibodies, and the epitopes are assumed to be contaminated with Protein A. An antibody is used to bind Protein A at a pH where the association is strong. The first step is to spike the protein solution with an appropriate IgG antibody, preferably free of biotin. It should also be from a different family than the antibody that will be used in the assay generally. The antibody is incubated 30 minutes with the protein solution, typically in a PBS pH 7.4 solution. Then filtered and the filtrate collected using a 100 kD membrane filter (e.g. Amicon 100 kD filters). The Protein A at 47 kD would normally pass the filter, but will not pass with the antibody to which it is bound. The protein of interest passes this filter and is collected, free of Protein A. The collected protein is dialyzed against PBS pH 7.4.

A typical example would be CFP10ESAT6 which is about 27 kD. A solution of 500 uG/mL of the fusion protein 1 ml volume is spiked with 50 uL of Goat antimouse IgG antibody 0.7 uG/mL and allowed to incubate 30 minutes. The solution is transferred to an Amicon 100 kD filter, placed on the centrifuge and filtrate collected. Recovery filtrate volume will be about 1 ml. The filtrate is transferred to a 1 mL dialysis tube 10 kD membrane and placed in a 1 L 10 mM PBS pH 7.4 solution and dialyzed with 3 exchanges over 24 hours. The freshly isolated fusion protein is then conjugated to ferrite beads in a modified protocol.

Removal of Protein A from Buffers and Other Reagents

Many commercial reagents are contaminated with Protein A. All reagents are filtered through a 30 kD Amicon filter before activating conjugation steps to be certain that Protein A does not inadvertently get back into the mix at these points. For example, in the MES washing steps of the ferrite beads before conjugation, and in preparing EDC and NHS reagents for bead activation the MES is pre-filtered. Filtered solutions or clean solutions are also used for quenching and then stabilizing the beads

Variability in Sera Samples

One difficulty in developing the Mbv assay concerned marked variability in sample analysis. Even when the same sample was repeatedly tested, considerable variance in the relative luminosity compared to a pool of negative controls was frequently observed. Many technical modifications and method improvements were initiated to minimize the variance issue but still there was poor reproducibility in terms of an absolute RL value for any given sample. However it was ultimately appreciated that the negative sera and positive sera behave differently in as far as positive sera are persistently well above the negative pool of sera used as a negative control, while the negative sera that test high do not consistently test high and can be eliminated by reflex supplemental testing.

In general it was found that very reproducible outcomes were achieved when a threshold for positive sera was set at approximately 1.6 standard deviations above the average for multiple negative sera tested. At this threshold in a simple, non-reflex screening (e.g., Table 5) the sensitivity was 88% and specificity 84%. All of the 99 samples were tested using randomization and blinding protocols to avoid any possible bias in the analysis. Some of the positive sera appeared very weakly positive, but were nevertheless detected both in the initial screen and on supplemental reflex testing. A comparison between initial screening and supplemental reflex testing results is shown in Table 5. It is important to appreciate that in very low prevalent states any test with less than 100% specificity will result in many false positives rendering the positive predictive value nearly useless. By adopting a reflex supplemental testing strategy, with each pass the pool of animals tested was enriched and concentrated providing the sensitivity was also high. This makes it possible to eliminate false positives with confidence in identifying truly infected animals.

Some of the determinants that optimized the assay relate to the use of 0.05% Tween in the wash solution and use of Lobind eppendorf tubes. Very low but unpredictable amounts of absorption onto the surface of the plastic containers contributed to variability and this could be reduced in the Lobind format. Considerable effort was directed to find evidence for possible bead loss but it appears that this was not a significant factor in the variance observed.

ROC Curves

The data may also be analyzed by another statistical application called the ROC (Receiver Operating Characteristic) curve, which takes into account variation in Sensitivity and Specificity as the arbitrary test threshold position is varied. How useful a test is at discriminating between two populations of true negatives and true positives is characterized by examining the area under the ROC curve. The closer the area is to 0.5, the worse the test and the closer it is to 1.0, the better the test. The area under the ROC curve after one initial screening using the Rapid Diagnostic test disclosed herein is 0.94 (FIG. 16).

Positive Predictive Value and Prevalence

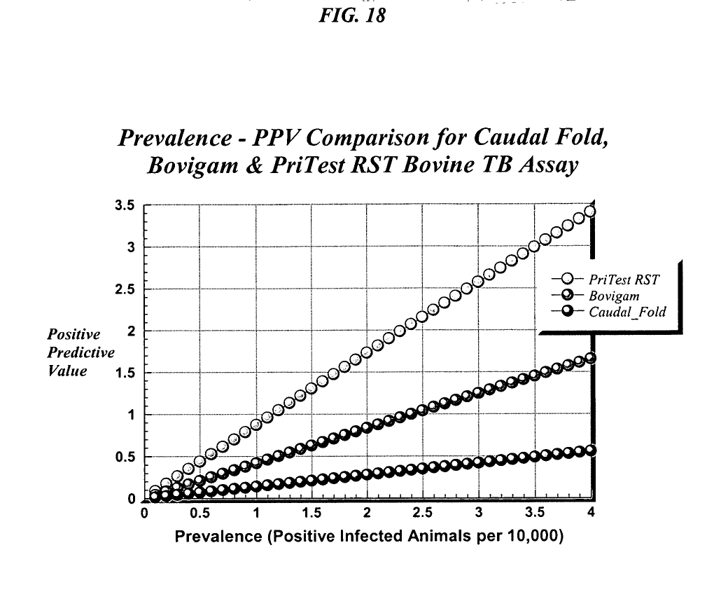

Using the reported Sensitivity and Specificity values for Caudal Fold, Bovigam, and the presently disclosed assay results in 99 randomized cattle sera tested using reflex supplemental testing, a direct comparison of Positive Predicted Values (PPV) based on the method used is shown below (FIG. 18). The test with the higher PPV for a given prevalence will predict more precisely which animals are infected. Positive Predicted Value is calculated by the formula;

$$\frac{\begin{matrix}Total True Positive Sera in Group - \\ Non Detected True Positives\end{matrix}}{Total Positives Detected in the Study}$$

The superiority of the presently claimed methods over existing assays—the Bovigam and Caudal fold—in positive predictive value is apparent from FIG. 18. With reflex supplemental testing, because of the high specificity (99%) it is anticipated that most of the false positives expected in a low prevalent testing environment will be released, rather than incorrectly slaughtered, as indicated by the reduction in number of false positive results with the reflex supplemental testing method disclosed herein.

### CONCLUSIONS

A rapid (less than 4 hour) method of differentiating between positively infected cattle with Mycobacterium bovis and uninfected cattle using a recombinant is disclosed herein. Screening of 99 samples yielded a sensitivity of 88% and specificity of 84%. By adjusting the threshold the test can be set to capture 100% of positively infected animals with a specificity of about 74%.

Reflex supplemental testing results in an improved outcome by reducing or eliminating false positive results. It requires only a minimal number of additional tests to increase the specificity to 99% without any change in the sensitivity (88%). Based on PPV, the disclosed reflex supplemental testing method is superior to either the Bovigam or Caudal fold test, which are the most accurate tests presently known in the art. Because it is faster and far less expensive and not technically demanding to conduct, it is a useful tool for eradicating M. bovis in cattle.

Serum is the only sample needed so a single visit and sample can provide the needed information to determine if the animal is infected. Serum can be stored frozen for an indefinite period of time until ready to commence testing. Repeat testing can be done as is deemed appropriate without any concern of having altered the animal's immune response in the sampling of blood.

### Example 2

Method of Detecting Pathogens

The present methods, compositions and apparatus allow the effective and rapid identification and isolation of tuberculosis infected animals, using a CP10_ESAT fusion protein assay. In such an assay, a synthetic protein or peptide, displaying one or more antigenic epitopes of Mycobacterium bovis, is allowed to react with blood, serum or plasma from an animal suspected of being infected with M. bovis. Binding of antibody to the target antigen indicates that the animal is infected with M. bovis. As the CP10_ESAT fusion protein used displays immune cross-reactivity with Mycobacterium species known to infect other species, such as humans, the skilled artisan will realize that the same compositions, apparatus and methods may be used to detect tuberculosis in other species, such as humans, bison, deer or any other animal known to be a potential carrier for Mycobacterium sp. Proteins homologous to CP10 and ESAT are well known antigens for various Mycobacterium species (e.g., van Pittius et al., Genome Biology 2(10), 2001). With the CP10_ESAT fusion protein attached to a substrate, such as a protein chip, Grabber™ slides or magnetic beads, the anti-Mycobacterium antibodies present in infected host blood will also bind to the substrate. After appropriate wash steps, the presence of anti-Mycobacterium antibodies in a sample may be detected, for example, by addition of commercially available biotinylated anti-bovine antibodies, followed by chemiluminescent assay using conjugated HRP-SA, peroxide and luminol as discussed above.

A preliminary set of 33 samples of bovine serum were tested in a blinded study, using the protocols disclosed above. Under the conditions of the assay, there were zero false negative results. Out of 33 blinded samples, there were 4 false positive results obtained on the first round of assays. Each sample that tested positive on the first round was subjected to reflex retesting, using the same assay at higher sensitivity. After reflex retesting the number of false positives declined to zero. An ROC curve analyzing sensitivity vs. specificity illustrates that the assay can obtain essentially 100% sensitivity at a specificity of about 70%, while 100% specificity is obtained at a sensitivity of about 70% (see, e.g., Table 7 and FIG. 16). By performing the reflex sample testing at 100% sensitivity and 70% specificity, it is possible to eliminate virtually all false positive test results.

The skilled artisan will realize that the reflex supplemental testing method disclosed herein is not limited to detection of tuberculosis, but may be used to detect virtually any type of pathogen, contaminant, biohazard, biowarfare agent, diseased cell or other condition, so long as an appropriate ligand and/or probe may be obtained. In this particular exemplary embodiment, the method discloses performing supplemental reflex testing on all positive samples, using the same assay at conditions of about 100% sensitivity and about 70% specificity. Under these conditions, there are virtually no false negative results. Therefore, each round of iterative reflex testing will eliminate about 70% of true negative subjects from the positive test pool. Use of 4 rounds of iterative reflex testing, applied to only those samples testing positive on the previous round, should result in a close to 100% accuracy in detecting infected animals. For example, 4 cycles of iterative testing at a 70% specificity would result in a false positive rate of 0.3×0.3×0.3×0.3, or less than 1%. Use of 5 cycles would result in about a 0.25% false positive rate. Use of 6 cycles would result in an false positive rate of less than 0.1%. Further, because the reflex testing is only performed on positive samples, the number of assays performed diminishes rapidly with each cycle, resulting in a rapid, efficient and inexpensive method to achieve close to 100% testing accuracy. The reflex supplemental testing strategy provides within a few hours a clear path to infected animals and they never re-enter the herd to infect others.

In the developed countries, such as the US and Europe, the percentage of infected animals in a herd is relatively low. Assuming an infection rate of 1 in 1,000,

| Event | Observations |
|---|---|
| 1000 Cattle possibly | 1 MB infected animal |
| 1 infected with MB | 999 non-infected animals |
| Test Results for 98% | 20 “false positive” |
| Sensitivity and Specificity | 49 of every 50 true positives detected |
| Expected Outcome | 21 animals all test positive |
| | Must use confirmatory tests to find the one |
| | animal infected, assuming he is not back in |
| | the herd infecting others. |

Suppose an assay was used that in the laboratory setting could consistently produce a 98% Sensitivity and 98% Specificity. That would by most standards be considered quite good. But in the above example where many negative animals are screened to find a single infected animal (1 in 1000 animals infected), we would expect 20 of the non-infected animals to test false positive. We see that high Sensitivity and Specificity alone will not do the job. We would expect to measure 21 animals positive in that group, assuming the infected animal didn't slip quietly back in with the herd undetected as a false negative. One in every 50 positive animals would escape to re-infect the herd with a 98% Sensitivity. We don't know how to identify which one of the 21 is truly infected. So a far more expensive confirmatory test must then be done on these 21 animals to settle the issue.

If the herd size is 10,000 with a single infected animal, the first pass should provide 201 positives. And as the number of infected animals in the herd increase, a number of false negative test results will occur. Undetected animals will re-enter to infect others in the herd.

No single test will ever consistently produce the level of Sensitivity and Specificity required to effectively isolate infected animals with 100% certainty. Reflex supplemental testing involves testing deliberately at a threshold where false positives are expected because the threshold for detecting positives has been set sufficiently low to catch 100% of positively infected animals. By then testing in a second batch all of those animals that did not pass negative on the first run again in the same assay, a second batch of truly non-infected animals can be safely released. The process can continue in sequential steps until only the true positive isolates are confirmed and rapidly centers on infected animals. This method is in principle much more effectively than a single step assay.

If the Specificity is moderately high at the threshold where 100% Sensitivity can be expected, then large numbers of animals are released in each run. A single tube of sera is more than adequate and tests are only repeated on “positive” outcome animals. Because Sensitivity is 100%, an animal that tested positive in the first run and negative in the second or third run can be safely released as truly un-infected. The disclosed exemplary assay provides 70% Specificity at the threshold for 100% Sensitivity. We can calculate the numbers to get to our positive infected animal in the 1000 herd number. By illustration the calculations are shown for a 10,000 animal number.

| | Observations | |
|---|---|---|
| | Infected or | Released as |
| Reflex Supplemental Testing | “Positive” | “Negative” |
| 1000 Cattle possibly | 1 MB infected | 999 non-infected |
| 1 infected with MB | animal | |
| Initial Test Screen | 300 | 700 |
| Reflex # 1 | 90 | 210 |
| Reflex # 2 | 27 | 63 |
| Reflex # 3 | 8 | 19 |
| Reflex # 4 | 2 | 6 |
| Totals | 428 | 998 |
| | | test non- |
| | | infected |

The sera for each animal is retained and reflex tested only if it is positive in a test. The total number of tests to isolate 2 animals out of a herd size of 1000 is 1128 and less than 1 ml of sera for each animal is more than sufficient to complete all tests. Because so many animals are released with each pass, only 128 additional tests are required to isolate the infected animal to one of two possibilities. To screen and cull to 2 animals in a herd size of 10,000 it would require only 6 Reflex test procedures and 11,285 tests. We would expect to release 7,000 animals in the initial screen.

All of the COMPOSITIONS, METHODS and APPARATUS disclosed and claimed herein can be made and executed without undue experimentation in light of the present disclosure. While the compositions and methods of this invention have been described in terms of preferred embodiments, it will be apparent to those of skill in the art that variations may be applied to the COMPOSITIONS, METHODS and APPARATUS and in the steps or in the sequence of steps of the methods described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

TABLE 1
Non-limiting Exemplary Pathogens

Actinobacillus spp.
Actinomyces spp.
Adenovirus (types 1, 2, 3, 4, 5 et 7)
Adenovirus (types 40 and 41)
Aerococcus spp.
Aeromonas hydrophila
Ancylostoma duodenale
Angiostrongylus cantonensis
Ascaris lumbricoides
Ascaris spp.
Aspergillus spp.
Bacillus anthracis
Bacillus cereus
Bacteroides spp.
Balantidium coli
Bartonella bacilliformis
Blastomyces dermatitidis
Bluetongue virus
Bordetella bronchiseptica
Bordetella pertussis
Borrelia burgdorferi
Branhamella catarrhalis
Brucella spp.
B. abortus
B. canis,
B. melitensis
B. suis
Brugia spp.
Burkholderia mallei
Burkholderia pseudomallei
Campylobacter fetus subsp. fetus
Campylobacter jejuni
C. coli
C. fetus subsp. jejuni
Candida albicans
Capnocytophaga spp.
Chlamydia psittaci
Chlamydia trachomatis
Citrobacter spp.
Clonorchis sinensis
Clostridium botulinum
Clostridium difficile
Clostridium perfringens
Clostridium tetani
Clostridium spp.
Coccidioides immitis
Colorado tick fever virus
Corynebacterium diphtheriae
Coxiella burnetii
Coxsackievirus
Creutzfeldt-Jakob agent, Kuru agent
Crimean-Congo hemorrhagic fever
virus
Cryptococcus neoformans
Cryptosporidium parvum
Cytomegalovirus
Dengue virus (1, 2, 3, 4)
Diphtheroids
Eastern (Western) equine encephalitis
virus
Ebola virus
Echinococcus granulosus
Echinococcus multilocularis
Echovirus
Edwardsiella tarda
Entamoeba histolytica
Enterobacter spp.
Enterovirus 70
Epidermophyton floccosum,
Microsporum spp. Trichophyton spp.
Epstein-Barr virus
Escherichia coli, enterohemorrhagic
Escherichia coli, enteroinvasive
Escherichia coli, enteropathogenic
Escherichia coli, enterotoxigenic
Fasciola hepatica
Francisella tularensis
Fusobacterium spp.
Gemella haemolysans
Giardia lamblia
Giardia spp.
Haemophilus ducreyi
Haemophilus influenzae (group b)
Hantavirus
Hepatitis A virus
Hepatitis B virus
Hepatitis C virus
Hepatitis D virus
Hepatitis E virus
Herpes simplex virus
Herpesvirus simiae
Histoplasma capsulatum
Human coronavirus
Human immunodeficiency virus
Human papillomavirus
Human rotavirus
Human T-lymphotrophic virus
Influenza virus
Junin virus/Machupo virus
Klebsiella spp.
Kyasanur Forest disease virus
Lactobacillus spp.
Legionella pneumophila
Leishmania spp.
Leptospira interrogans
Listeria monocytogenes
Lymphocytic choriomeningitis virus
Marburg virus
Measles virus
Micrococcus spp.
Moraxella spp.
Mycobacterium spp.
Mycobacterium tuberculosis, M. bovis
Mycoplasma hominis, M. orale,
M. salivarium, M. fermentans
Mycoplasma pneumoniae
Naegleria fowleri
Necator americanus
Neisseria gonorrhoeae
Neisseria meningitidis
Neisseria spp.
Nocardia spp.
Norwalk virus
Omsk hemorrhagic fever virus
Onchocerca volvulus
Opisthorchis spp.
Parvovirus B19
Pasteurella spp.
Peptococcus spp.
Peptostreptococcus spp.
Plesiomonas shigelloides
Powassan encephalitis virus
Proteus spp.
Pseudomonas spp.
Rabies virus
Respiratory syncytial virus
Rhinovirus
Rickettsia akari
Rickettsia prowazekii, R. canada
Rickettsia rickettsii
Ross river virus/O'Nyong-Nyong
virus
Rubella virus
Salmonella choleraesuis
Salmonella paratyphi
Salmonella typhi
Salmonella spp.
Schistosoma spp.
Scrapie agent
Serratia spp.
Shigella spp.
Sindbis virus
Sporothrix schenckii
St. Louis encephalitis virus
Murray Valley encephalitis virus
Staphylococcus aureus
Streptobacillus moniliformis
Streptococcus agalactiae
Streptococcus faecalis
Streptococcus pneumoniae
Streptococcus pyogenes
Streptococcus salivarius
Taenia saginata
Taenia solium
Toxocara canis, T. cati
Toxoplasma gondii
Treponema pallidum
Trichinella spp.
Trichomonas vaginalis
Trichuris trichiura
Trypanosoma brucei
Ureaplasma urealyticum
Vaccinia virus
Varicella-zoster virus
Venezuelan equine encephalitis
Vesicular stomatitis virus
Vibrio cholerae, serovar 01
Vibrio parahaemolyticus
Wuchereria bancrofti
Yellow fever virus
Yersinia enterocolitica
Yersinia pseudotuberculosis
Yersinia pesti

TABLE 2
Predicted RST Number Dependence on Specificity and Prevalence
Reflex Supplemental Testing - Number Calculator

| Sample | | | | | | | | | | | | Total | Total |
|---|---|---|---|---|---|---|---|---|---|---|---|---|---|
| # | N | R1 | R2 | R3 | R4 | R5 | R6 | R7 | R8 | R9 | R10 | Tests | Tests |
| Specificity | | | | 0.99 | | | | | | | | | |
| Number Positive | | | | 1 | | | | | | | | | |
| Number False Positive | | | | 1 | | | | | | | | | |
| Specificity Factor (1-Sp) | | | | 0.01 | | | | | | | | | |
| 100 | 1 | 1 | 0 | 0 | 0 | 0 | 0 | 0 | 0 | 0 | 0 | 1 | 101 |
| 500 | 1 | 5 | 0 | 0 | 0 | 0 | 0 | 0 | 0 | 0 | 0 | 5 | 505 |
| 1000 | 2 | 10 | 0 | 0 | 0 | 0 | 0 | 0 | 0 | 0 | 0 | 10 | 1010 |
| 2000 | 2 | 20 | 0 | 0 | 0 | 0 | 0 | 0 | 0 | 0 | 0 | 20 | 2020 |
| 5000 | 2 | 50 | 0 | 0 | 0 | 0 | 0 | 0 | 0 | 0 | 0 | 50 | 5050 |
| 10000 | 2 | 100 | 0 | 0 | 0 | 0 | 0 | 0 | 0 | 0 | 0 | 100 | 10100 |
| 100000 | 3 | 1000 | 10 | 0 | 0 | 0 | 0 | 0 | 0 | 0 | 0 | 1010 | 101010 |
| 500000 | 3 | 5000 | 50 | 0 | 0 | 0 | 0 | 0 | 0 | 0 | 0 | 5050 | 505050 |
| Specificity | | | | 0.74 | | | | | | | | | |
| Specificity Factor (1-Sp) | | | | 0.26 | | | | | | | | | |
| 100 | 3 | 26 | 6 | 1 | 0 | 0 | 0 | 0 | 0 | 0 | 0 | 34 | 134 |
| 500 | 5 | 130 | 33 | 8 | 2 | 0 | 0 | 0 | 0 | 0 | 0 | 174 | 674 |
| 1000 | 5 | 260 | 67 | 17 | 4 | 0 | 0 | 0 | 0 | 0 | 0 | 348 | 1348 |
| 2000 | 6 | 520 | 135 | 35 | 9 | 2 | 0 | 0 | 0 | 0 | 0 | 700 | 2700 |
| 5000 | 6 | 1300 | 338 | 88 | 22 | 6 | 1 | 0 | 0 | 0 | 0 | 1754 | 6754 |
| 10000 | 7 | 2600 | 676 | 175 | 45 | 12 | 3 | 0 | 0 | 0 | 0 | 3510 | 13510 |
| 100000 | 9 | 26000 | 6760 | 1757 | 457 | 118 | 31 | 8 | 2 | 0 | 0 | 35132 | 135132 |
| 500000 | 10 | 130000 | 33800 | 8788 | 2285 | 594 | 154 | 40 | 10 | 2 | 0 | 175672 | 675672 |
| Specificity | | | | 0.70 | | | | | | | | | |
| Specificity Factor (1-Sp) | | | | 0.30 | | | | | | | | | |
| 100 | 4 | 30 | 9 | 2 | 0 | 0 | 0 | 0 | 0 | 0 | 0 | 41 | 141 |
| 500 | 5 | 150 | 45 | 13 | 4 | 0 | 0 | 0 | 0 | 0 | 0 | 211 | 711 |
| 1000 | 6 | 300 | 90 | 27 | 8 | 2 | 0 | 0 | 0 | 0 | 0 | 426 | 1426 |
| 2000 | 6 | 600 | 180 | 54 | 16 | 4 | 1 | 0 | 0 | 0 | 0 | 854 | 2854 |
| 5000 | 7 | 1500 | 450 | 135 | 40 | 12 | 3 | 0 | 0 | 0 | 0 | 2139 | 7139 |
| 10000 | 8 | 3000 | 900 | 270 | 81 | 24 | 7 | 2 | 0 | 0 | 0 | 4282 | 14282 |
| 100000 | 10 | 30000 | 9000 | 2700 | 810 | 243 | 72 | 21 | 6 | 2 | 0 | 42853 | 142853 |
| 500000 | 11 | 150000 | 45000 | 13500 | 4050 | 1215 | 364 | 109 | 32 | 9 | 3 | 214280 | 714280 |

TABLE 3
Alterative Color Filters for a CMOS Color Imager

| Wavelength Range (nm) | Filter Type | Modified Bayer Pattern |
|---|---|---|
| 510-810 | Yellow | Y, Y, Y, Y |
| 610-810 | Magenta | M, M, M, M |
| 490-550 | Cyan | C, C, C, C |
| 490-810 | Yellow, Cyan | Y, Y, Y, C |
| 350-550 | None | Monochrome pattern |

TABLE 4
Quantum Efficiency and Normalization Factors for
Kodak 1310 Image Sensor at 670 nm

| | Quantum Efficiency | |
|---|---|---|
| Filter Type | (%) | Normalization Factor |
| Yellow | 43 | 2.38 |
| Magenta | 44 | 2.27 |
| Cyan | 13 | 7.69 |
| Monochrome (no filter) | 28 | 3.57 |
| Red | 35 | 2.86 |
| Green | 5 | 20.0 |
| Blue | 3 | 33.3 |

TABLE 5
Bovine TB S&S Blinded Study Initial Screen

| | |
|---|---|
| Obs | Observed sample numeric value |
| BG | Background numeric value between samples |
| RL | Relative luminosity (Obs − BG) |
| Delta NC | Value of the difference (Obs − NC) |
| NC | Pooled sera negative control value (not included in table) |
| Pos | Sample assignment is positive if > NC threshold |
| Neg | Sample assignment is negative if < NC threshold |

| Sample | Obs | BG | RL | Delta NC | 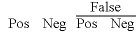 | Actual Neg Pos | Unblinded Assignments |
|---|---|---|---|---|---|---|---|
| 14-1 14-2 14-3 14-4 14-5 14-6 14-7 14-8 | 4034 4488 4651 3430 4170 4260 4556 3923 | 3226 3226 3226 3226 3226 3226 3226 3226 | 808 1262 1425 204 944 1034 1330 697 | −181 273 436 −785 | | | |
| # −45 45 341 −292 | 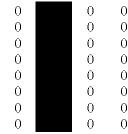 | 1 0 1 0 1 0 1 0 1 0 1 0 1 0 1 0 | Negative Negative Negative Negative Negative Negative Negative Negative | | | | |
| 14-9 14-10 14-11 14-12 14-13 14-14 14-15 | 5205 4708 4372 2041 1813 1116 1744 | 3226 3226 3226 840 840 840 840 | 1979 1482 1146 1201 973 276 904 | 990 493 157 594 366 −331 297 | 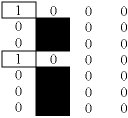 | 0 1 1 | |
| # 0 1 0 0 1 1 0 1 0 1 0 | Positive Negative Negative Positive Negative Negative Negative | | | | | | |
| 14-16 14-17 14-18 14-19 14-20 14-21 14-22 | 2208 2784 3508 2280 3173 2163 1853 | 840 840 840 840 840 840 840 | 1368 1944 2668 1440 2333 1323 1013 | 761 1337 2061 833 1726 716 406 | 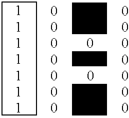 | 1 0 | |
| # 1 0 0 1 1 0 0 1 1 0 1 0 | Negative Negative Positive Negative Positive Negative Negative | | | | | | |
| 14-23 14-24 14-25 14-26 14-27 14-28 14-29 14-30 14-31 14-32 14-33 | 4291 4582 5308 4279 5016 5293 5176 5245 4681 4769 5982 | 3713 3713 3713 3713 3713 3713 3713 3713 3713 3713 3713 | 578 869 1595 566 1303 1580 1463 1532 | | | | |
| # 968 1056 2269 | −418 −127 599 −430 307 584 467 536 −28 60 1273 | 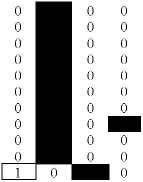 | 1 0 1 0 1 0 1 0 1 0 1 0 1 0 0 1 | | | | |
| # 1 0 1 0 1 0 | Negative Negative Negative Negative Negative Negative Negative Positive Negative Negative Negative | | | | | | |
| 16-1 16-2 16-3 16-4 16-5 16-6 16-7 | 3179 2561 3704 2981 3352 4816 3760 | 2198 2198 2198 2198 2198 2198 2198 | 981 363 1506 783 1154 2618 1562 | 182 −436 707 −16 355 1819 763 | 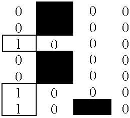 | 1 0 | |
| # 1 0 0 1 1 0 1 0 0 1 1 0 | Negative Negative Positive Negative Negative Positive Negative | | | | | | |
| 16-8 16-9 16-10 16-11 16-12 16-13 16-14 16-15 | 3452 2952 3466 4256 2843 3719 3705 2908 | 2198 2198 2198 2198 2058 2058 2058 2058 | 1254 754 1268 2058 785 1661 1647 850 | 455 −45 469 1259 | | | |
| # −282 594 580 −217 | 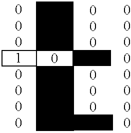 | 1 0 1 0 1 0 1 0 1 0 1 0 1 0 1 0 | Negative Negative Negative Negative Negative Negative Negative Negative | | | | |
| 16-16 16-17 16-18 16-19 16-20 | 4794 3438 3540 2844 4508 | 2058 2058 2058 2058 2058 | 2736 1380 1482 786 2450 | 1669 313 415 −281 1383 | 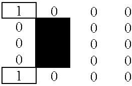 | 0 1 1 0 1 0 1 0 0 1 | Positive |
| # Negative Negative Negative Positive | | | | | | | |
| 16-21 16-22 16-23 16-24 16-25 16-26 16-27 16-28 16-29 | 3120 2977 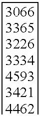 | 2058 2058 2188 2188 2188 2188 2188 2188 2188 | 1062 919 878 1177 1038 1146 2405 1233 2274 | −5 −148 −68 231 92 200 1459 287 1328 | 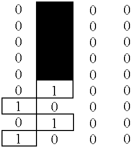 | 1 | |
| # 0 1 0 1 0 1 0 1 0 1 0 0 1 1 0 0 1 | Negative Negative Negative Negative Negative Negative Positive Negative Positive | | | | | | |
| 16-30 16-31 16-32 16-33 | 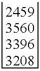 | 2188 2188 2188 2188 | 271 1372 1208 1020 | −675 426 262 74 | 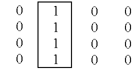 | 1 0 1 0 1 0 1 0 | Negative Negative Negative Negative |
| 17-1 17-2 17-3 17-4 17-5 17-6 17-7 | 4080 4182 4164 4014 4020 4331 4581 | 3557 3557 3557 3557 3557 3557 3557 | 523 625 607 457 463 774 1024 | 35 137 119 −31 −25 286 536 | 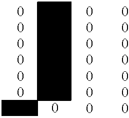 | 1 | |
| # 0 1 0 1 0 1 0 1 0 1 0 0 1 | Negative Negative Negative Negative Negative Negative Positive | | | | | | |
| 17-8 17-9 17-10 17-11 17-12 17-13 17-14 | 3704 3920 4162 4499 5451 4511 4088 | 3557 3557 3557 3557 3510 3510 3510 | 147 363 605 942 1941 1001 578 | −341 −125 117 454 1195 255 | | | |
| # −168 | 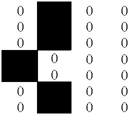 | 1 0 1 0 1 0 0 1 0 1 1 0 1 0 | Negative Negative Negative Positive Positive Negative Negative | | | | |
| 17-15 17-16 17-17 17-18 17-19 17-20 | 5142 4227 4039 4914 4515 4742 | 3510 3510 3510 3510 3510 3510 | 1632 717 529 1404 1005 1232 | 886 −29 −217 658 259 486 | 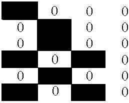 | 0 1 1 | |
| # 0 1 0 1 0 1 0 1 0 | Positive Negative Negative Negative Negative Negative | | | | | | |
| 17-21 17-22 17-23 17-24 17-25 17-26 17-27 | 4222 4396 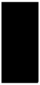 | 3510 3510 3442 3442 3442 3442 3442 | 712 886 831 1232 901 927 1047 | −34 140 89 490 159 185 305 | 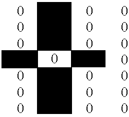 | 1 0 1 0 | |
| # 1 0 1 0 1 0 1 0 1 0 | Negative Negative Negative Negative Negative Negative Negative | | | | | | |
| 17-28 17-29 17-30 17-31 17-32 17-33 | 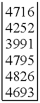 | 3442 3442 3442 3442 3442 3442 | 1274 810 549 1353 1384 1251 | 532 68 −193 611 642 509 | 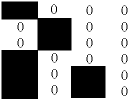 | 0 1 1 0 1 0 0 1 | |
| # 1 0 1 0 | Positive Negative Negative Positive Negative Negative | | | | | | |
| | | | | | 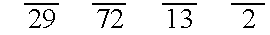 | \overline{82 17} | Totals |

| | |
|---|---|
| Sensitivity | 94 |
| Specificity | 84 |
| ROC Area | 0.94 |
| PPV | 55 |
| NPV | 99 |

TABLE 6
Negative Control Reference Luminosity for 9 Crib Sets

| NC | BG | NC_RL | {Constant − 1} | Threshold | Crib Set |
|---|---|---|---|---|---|
| 4215 | 3226 | 989 | 0.62 | 613 | 1 |
| 1447 | 840 | 607 | 0.62 | 376 | 2 |
| 4709 | 3713 | 996 | 0.62 | 618 | 3 |
| 2997 | 2198 | 799 | 0.62 | 495 | 4 |
| 3125 | 2058 | 1067 | 0.62 | 662 | 5 |
| 3134 | 2188 | 946 | 0.62 | 587 | 6 |
| 4045 | 3557 | 488 | 0.62 | 303 | 7 |
| 4256 | 3510 | 746 | 0.62 | 463 | 8 |
| 4184 | 3442 | 742 | 0.62 | 460 | 9 |

TABLE 7
ROC Plot for 99 Samples Positive and Negative for M. Bovis.¹

| | | Threshold |
|---|---|---|
| Specificity (%) | Sensitivity (%) | Factor |
| 66 | 100 | 0.4 |
| 74 | 100 | 0.5 |
| 82 | 94 | 0.6 |
| 84 | 94 | 0.62 |
| 85 | 94 | 0.66 |
| 88 | 94 | 0.68 |
| 89 | 94 | 0.7 |
| 89 | 88 | 0.8 |
| 91 | 76 | 0.9 |
| 93 | 65 | 1 |
| 93 | 53 | 1.1 |
| 94 | 47 | 1.2 |
| 96 | 41 | 1.3 |
| 98 | 41 | 1.4 |

¹False Positive % is 100% - Specificity.

TABLE 8
Bovine TB S&S Blinded Study Summary

| Sample | Obs | BG | RL | Delta NC | 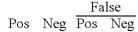 | Actual Neg Pos | Unblinded Assignments |
|---|---|---|---|---|---|---|---|
| 14-1 14-2 14-3 14-4 14-5 14-6 14-7 14-8 | 4034 4488 4651 3430 4170 4260 4556 3923 | 3226 3226 3226 3226 3226 3226 3226 3226 | 808 1262 1425 204 944 1034 1330 697 | −181 273 436 −785 | | | |
| # −45 45 341 −292 | 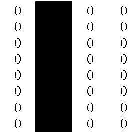 | 1 0 1 0 1 0 1 0 1 0 1 0 1 0 1 0 | Negative Negative Negative Negative Negative Negative Negative Negative | | | | |
| 14-9 14-10 14-11 14-12 14-13 14-14 14-15 14-16 14-17 | 5513 4708 4372 5690 1813 1116 1744 5692 5659 | 3711 3226 3226 3711 840 840 840 3749 3749 | 1802 1482 1146 1979 973 276 904 1943 1910 | 737 4113 157 | | | |
| # 914 366 −331 297 741 708 | 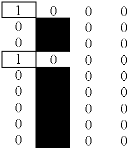 | 0 1 1 0 1 0 0 1 1 0 1 0 1 0 1 0 1 0 | Positive Negative Negative Positive Negative Negative Negative Negative Negative | | | | |
| 14-18 14-19 14-20 14-21 | 5729 5482 6545 5264 | 3711 3711 3711 3749 | 2018 1771 2834 1515 — | 953 706 1769 313 | 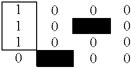 | 0 1 1 0 0 1 1 0 | Positive Negative Positive Negative |
| 14-22 14-23 14-24 14-25 14-26 14-27 14-28 | 0 4291 4582 5308 4279 5016 52113 | 3749 3713 3713 3713 3713 3713 3713 | 3749 578 869 1595 566 1303 1580 | −4951 −418 −127 599 −430 307 584 | 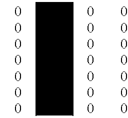 | 1 0 | |
| # 1 0 1 0 1 0 1 0 1 0 1 0 | Negative Negative Negative Negative Negative Negative Negative | | | | | | |
| 14-29 14-30 14-31 14-32 14-33 | 5176 5245 4681 4769 5425 | 3713 3713 3713 3713 3711 | 1463 1532 968 1056 1714 | 467 536 −28 60 649 | 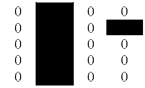 | 1 0 | |
| # 0 1 1 0 1 0 1 0 | Negative Positive Negative Negative Negative | | | | | | |
| 16-1 16-2 16-3 16-4 16-5 16-6 | 3179 2561 10000 2981 3352 5346 | 2198 2198 3477 2198 2198 3477 | 981 363 6523 783 1154 1869 | 182 −436 | | | |
| # 6026 −16 355 1372 | 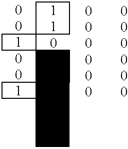 | 1 0 1 0 0 1 1 0 1 0 0 1 | Negative Negative Positive Negative Negative Positive | | | | |
| 16-7 16-8 16-9 16-10 16-11 16-12 16-13 16-14 16-15 | 4043 3452 2952 3466 4161 2843 3719 3705 2908 | 3391 2198 2198 2198 3391 2058 2058 2058 2058 | 652 1254 754 1268 | | | | |
| # 770 785 1661 1647 850 | 157 455 −45 469 275 −282 594 580 −217 | 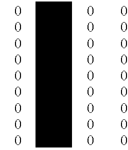 | 1 0 1 0 1 0 1 0 1 0 1 0 1 0 | | | | |
| # 1 0 1 0 | Negative Negative Negative Negative Negative Negative Negative Negative Negative | | | | | | |
| 16-16 16-17 16-18 16-19 16-20 | 6229 3438 3540 2844 100000 | 3477 2058 2058 2058 3477 | 2752 1380 1482 786 96523 | 2255 313 415 −281 96026 | 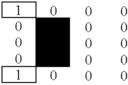 | 0 1 1 0 1 0 | |
| # 1 0 0 1 | Positive Negative Negative Negative Positive | | | | | | |
| 16-21 16-22 16-23 16-24 16-25 16-26 | 3120 2977 3066 3365 3226 3334 | 2058 2058 2188 2188 2188 2188 | 1062 919 878 1177 1038 1146 | −5 −148 −68 231 92 200 | 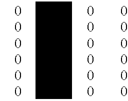 | 1 0 1 0 1 0 | |
| # 1 0 1 0 1 0 | Negative Negative Negative Negative Negative Negative | | | | | | |
| 16-27 16-28 16-29 16-30 16-31 16-32 16-33 | 5443 3421 6118 2459 3560 3396 3208 | 3477 2188 3477 2188 2188 2188 2188 | 1966 1233 2641 271 1372 1208 1020 | 1469 287 2144 −675 426 262 74 | 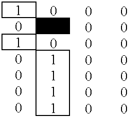 | 0 1 1 0 | |
| # 0 1 1 0 1 0 1 0 1 0 | Positive Negative Positive Negative Negative Negative Negative | | | | | | |
| 17-1 17-2 17-3 17-4 17-5 17-6 17-7 17-8 17-9 | 4080 4182 4164 4014 4020 4331 4455 3704 3920 4162 | 3557 3557 3557 3557 3557 3557 3511 3557 3557 3557 | 523 625 607 457 463 774 944 147 363 | | | | |
| # 605 | 35 137 119 −31 −25 286 181 −341 −125 117 | 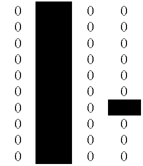 | 1 0 1 0 1 0 1 0 1 0 1 0 0 1 1 0 1 0 | | | | |
| # 1 0 | Negative Negative Negative Negative Negative Negative Positive Negative Negative Negative | | | | | | |
| 17-11 17-12 17-13 17-14 17-15 | 10000 4115 4511 4088 4054 | 2203 2203 3510 3510 2203 | 7797 1912 1001 578 1851 | 7250 1365 255 −168 1304 | 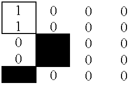 | 0 1 0 1 1 0 1 0 0 1 | Positive Positive Negative Negative 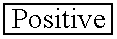 |
| 17-16 17-17 17-18 17-19 | 4227 4039 4324 4515 | 3510 3510 3511 3510 | 717 529 813 1005 — | −29 −217 50 259 | 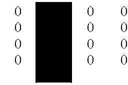 | 1 0 1 0 1 0 1 0 | Negative Negative Negative Negative |
| 17-20 17-21 17-22 17-23 | 0 4222 4396 4273 | 3511 3510 3510 3442 | 3511 712 886 831 — | −4274 −34 140 89 | 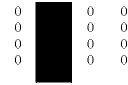 | 1 0 1 0 1 0 1 0 | Negative Negative Negative Negative |
| 17-24 17-25 17-26 17-27 17-28 | 0 4343 4369 4489 3684 | 3511 3442 3442 3442 2203 | 3511 901 927 1047 1481 | −4274 159 185 305 894 | 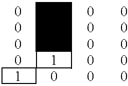 | 1 0 1 0 1 0 1 0 | |
| # 0 1 | Negative Negative Negative Negative Positive | | | | | | |
| 17-29 17-30 17-31 17-32 17-33 | 4252 3991 3698 4166 4373 | 3442 3442 2203 3511 3511 | 810 549 1495 655 862 | 68 −1113 908 −108 99 | 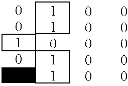 | 1 0 1 0 0 1 1 0 | |
| # 1 0 | Negative Negative 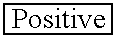 Negative Negative | | | | | | |
| | | | | | 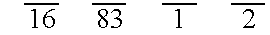 | \overline{82 17} | |

| | |
|---|---|
| Sensitivity | 88 |
| Specificity | 99 |
| PPV | 94 |
| NPV | 96 |

TABLE 9
Modified and Unusual Amino Acids

| Abbr. | Amino Acid | Abbr. | Amino Acid |
|---|---|---|---|
| Aad | 2-Aminoadipic acid | EtAsn | N-Ethylasparagine |
| Baad | 3-Aminoadipic acid | Hyl | Hydroxylysine |
| Bala | β-alanine, β-Amino-propionic acid | AHyl | allo-Hydroxylysine |
| Abu | 2-Aminobutyric acid | 3Hyp | 3-Hydroxyproline |
| 4Abu | 4-Aminobutyric acid, piperidinic | 4Hyp | 4-Hydroxyproline |
| | acid | | |
| Acp | 6-Aminocaproic acid | Ide | Isodesmosine |
| Ahe | 2-Aminoheptanoic acid | AIle | allo-Isoleucine |
| Aib | 2-Aminoisobutyric acid | MeGly | N-Methylglycine, |
| | | | sarcosine |
| Baib | 3-Aminoisobutyric acid | MeIle | N-Methylisoleucine |
| Apm | 2-Aminopimelic acid | MeLys | 6-N-Methyllysine |
| Dbu | 2,4-Diaminobutyric acid | MeVal | N-Methylvaline |
| Des | Desmosine | Nva | Norvaline |
| Dpm | 2,2′-Diaminopimelic acid | Nle | Norleucine |
| Dpr | 2,3-Diaminopropionic acid | Orn | Ornithine |
| EtGly | N-Ethylglycine | | |

TABLE 10
Comparison between Screening and RST Test Results

| | Screening | RST |
|---|---|---|
| Sensitivity | 94 | 88 |
| Specificity | 84 | 99 |
| PPV | 55 | 94 |
| NPV | 99 | 96 |
| Area under ROC | 0.94 | n/a |

PPV: positive predictive value;
NPV: negative predictive value.

What is claimed is:

1. A method for detecting a pathogen in a group of samples comprising:
a) assaying the samples for the presence of the pathogen at a sensitivity of about 100%;
b) using the same assay to iteratively retest only those samples that show positive test results; and
c) repeating the iterative retesting on only those samples that show positive test results for each round of testing, until a selective level of accuracy is obtained.

2. The method of claim 1, wherein the iterative retesting is repeated for three, four, five or six cycles.

3. The method of claim 1, wherein the assay has a sensitivity of 100% and a selectivity of 70%.

4. The method of claim 1, wherein the assay has a sensitivity of 99%, 99.5%, 99.8%, 99.9% or 100%.

5. The method of claim 1, wherein the pathogen is a species Mycobacterium.

6. The method of claim 5, wherein the pathogen is Mycobacterium bovis.

7. The method of claim 5, wherein the assay comprises exposing a CP10_ESAT fusion protein to a sample of blood, serum or plasma from a subject and detecting antibody binding to the fusion protein.

8. The method of claim 7, wherein the subject is a cow, a badger, a bison, a deer or a human.

9. The reflex supplemental testing method of claim 1, wherein the number of false negative results is zero.

10. The method of claim 7, further comprising detecting the presence of anti-fusion protein antibody in a sample using biotinylated goat IgG anti-bovine IgM antibody, horseradish peroxidase conjugated streptavidin and a luminal peroxide solution to generate chemiluminescence.

11. The method of claim 10, wherein chemiluminescence is measured using a Total Optical Assay Device.

12. The method of claim 10, further comprising performing data analysis on the measured chemiluminescent signal from each sample.

13. The method of claim 12, wherein the data analysis comprises an auto-threshold correction.

14. The method of claim 12, wherein the data analysis comprises a background determination for groups of pixels, the background for the group of pixels set to equal the highest background emission for any pixel in the group.

15. The method of claim 12, wherein the data analysis comprises applying a quantum efficiency correction factor.

16. The method of claim 13, wherein the threshold for a positive result is set at about 1.6 standard deviations above the average test value for multiple negative samples.

17. The method of claim 7, wherein the fusion protein is conjugated to magnetic beads.

18. The method of claim 11, further comprising using spherical magnets to collect the magnetic beads from solution.

19. A method for detecting a molecular marker in a sample comprising:
a) assaying the sample for the presence of the molecular marker at a sensitivity of about 100%;
b) using the same assay to iteratively retest only those samples that show positive test results; and
c) repeating the iterative retesting on only those samples that show positive test results after each round of testing, until a selective level of accuracy is obtained.

20. The method of claim 19, wherein the molecular marker is a marker for a disease state.


SEQUENCE LISTING (6 sequences)

SEQ ID NO: 1 - protein, 100 aa, Mycobacterium tuberculosis
MAEMKTDAATLAQEAGNFERISGDLKTQIDQVESTAGSLQGQWRGAAGTAAQAAVVRFQEAANKQKQELDEISTNIRQAGVQYSRADEEQQQALSSQMGF

SEQ ID NO: 2 - DNA, 303 bp, Mycobacterium tuberculosis
atggcagagatgaagaccgatgccgctaccctcgcgcaggaggcaggtaatttcgagcggatctccggcgacctgaaaacccagatcgaccaggtggagtcgacggcaggttcgttgcagggccagtggcgcggcgcggcggggacggccgcccaggccgcggtggtgcgcttccaagaagcagccaataagcagaagcaggaactcgacgagatctcgacgaatattcgtcaggccggcgtccaatactcgagggccgacgaggagcagcagcaggcgctgtcctcgcaaatgggcttctga

SEQ ID NO: 3 - DNA, 32 bp, Artificial
  synthetic oliganucleotide
cccgctaatacgaaaagaaacggagcaaaaac

SEQ ID NO: 4 - protein, 95 aa, Mycobacterium tuberculosis
MTEQQWNFAGIEAAASAIQGNVTSIHSLLDEGKQSLTKLAAAWGGSGSEAYQGVQQKWDATATELNNALQNLARTISEAGQAMASTEGNVTGMFA

SEQ ID NO: 5 - DNA, 288 bp, Mycobacterium tuberculosis
atgacagagcagcagtggaatttcgcgggtatcgaggccgcggcaagcgcaatccagggaaatgtcacgtccattcattccctccttgacgaggggaagcagtccctgaccaagctcgcagcggcctggggcggtagcggttcggaggcgtaccagggtgtccagcaaaaatgggacgccacggctaccgagctgaacaacgcgctgcagaacctggcgcggacgatcagcgaagccggtcaggcaatggcttcgaccgaaggcaacgtcactgggatgttcgcatag

SEQ ID NO: 6 - DNA, 624 bp, Mycobacterium tuberculosis
atggcagagatgaagaccgatgccgctaccctcgcgcaggaggcaggtaatttcgagcggatctccggcgacctgaaaacccagatcgaccaggtggagtcgacggcaggttcgttgcagggccagtggcgcggcgcggcggggacggccgcccaggccgcggtggtgcgcttccaagaagcagccaataagcagaagcaggaactcgacgagatctcgacgaatattcgtcaggccggcgtccaatactcgagggccgacgaggagcagcagcaggcgctgtcctcgcaaatgggcttcggacccgctgaatacgaaaagaaacggagcaaaaacatgacagagcagcagtggaatttcgcgggtatcgaggccgcggcaagcgcaatccagggaaatgtcacgtccattcattccctccttgacgaggggaagcagtccctgaccaagctcgcagcggcctggggcggtagcggttcggaggcgtaccagggtgtccagcaaaaatgggacgccacggctaccgagctgaacaacgcgctgcagaacctggcgcggacgatcagcgaagccggtcaggcaatggcttcgaccgaaggcaacgtcactgggatgttcgcatag